(19) United States
(12) Patent Application Publication
(10) Pub. No.: US 2012/0010138 A1
(43) Pub. Date: Jan. 12, 2012

(54) NOVEL USES OF VEGFXXXB

(76) Inventors: David Owen Bates, Bristol (GB); Steven James Harper, Bristol (GB); Andrew Salmon, Bristol (GB)

(21) Appl. No.: 13/113,643

(22) Filed: May 23, 2011

Related U.S. Application Data
(63) Continuation-in-part of application No. PCT/GB2009/051591, filed on Nov. 23, 2009.

(30) Foreign Application Priority Data
Nov. 22, 2008 (GB) 0821412.4
Mar. 27, 2009 (GB) 0905280.4

(51) Int. Cl. A61K 38/18; C12Q 1/68; G01N 33/566; A61P 9/00; C12N 5/071

(52) U.S. Cl. 514/8.1; 435/375; 435/7.1; 435/7.92; 435/6.11

(57) ABSTRACT

The invention provides VEGFₓₓₓb, or an agent which selectively promotes the expression of VEGFₓₓₓb in preference to VEGFₓₓₓ in cells of a subject or in vitro, or an expression vector system which causes the expression of the VEGFₓₓₓb in a host organism, for use in treating or preventing microvascular hyperpermeability disorders, or in regulating the pro-angiogenic pro-permeability properties of VEGFₓₓₓ isoforms, or in supporting epithelial cell survival without increased permeability, or in reducing the nature (for example the number density and/or size) of fenestrations of epithelial filtration membranes in vivo or in vitro.

### CROSS-REFERENCE TO RELATED APPLICATIONS

This application is a continuation-in-part of International Serial No. PCT/GB2009/051591, filed Nov. 23, 2009, which claims priority of GB Patent Application No. 0905280.4, filed Mar. 27, 2009, and GB Patent Application No. 0821412.4, filed Nov. 22, 2008. The entire disclosures of the applications identified in this paragraph are incorporated herein by references.

### FIELD

The present invention relates to the use of VEGFₓₓₓb against microvascular hyperpermeability disorders, disorders of regulation of the pro-angiogenic pro-permeability properties of VEGFₓₓₓ isoforms, disorders of epithelial cell survival with or without increased permeability, and disorders of the nature (for example the number density and/or size) of fenestrations of epithelial filtration membranes.

The present invention also relates to corresponding uses of agents that promote the endogenous expression of VEGFₓₓₓb by the subject having, or at risk of, microvascular hyperpermeability disorders, disorders of regulation of the pro-angiogenic pro-permeability properties of VEGFₓₓₓ isoforms, disorders of epithelial cell survival and permeability, and disorders of the nature (for example the number density and/or size) of fenestrations of epithelial filtration membranes, via alternative splicing of the VEGF-A gene.

### BACKGROUND

The prior publications referred to below are listed at the end of the description of the invention and are each incorporated herein by reference to the extent prescribed by the law applicable to this application and subsequent patents in each jurisdiction.

Vascular Endothelial Growth Factor-A (VEGF-A) is a potent angiogenic factor that induces endothelial cell migration, proliferation, differentiation and regeneration [1]. In kidneys of embryos to adults, VEGF-A is expressed in presumptive and mature glomerular epithelial cells (podocytes) and tubular epithelial cells [2-7].

Normal glomerulogenesis requires the coordinated induction of epithelial differentiation, endothelial invasion, and growth of tubular and vascular tissues. In mice, (which have one less amino acid in the VEGF proteins than in human) specific overexpression or deletion of the VEGF-A gene in podocytes results in glomerular dysfunction [8,9]. A podocyte specific cre-recombinase knockout of even a single gene copy leads to nephrotic syndrome, uraemia and death 9 weeks post-partum whilst complete knockouts died a few hours post-partum [8]. In mice, glomerular overexpression of the most widely studied isoform of VEGF-A, VEGF₁₆₄, results in death a few days post-partum with renal haemorrhages, [8]. In VEGF inhibition studies, murine pups treated at postnatal day 0 with VEGF-blocking antibodies, exhibit marked glomerular abnormalities, with many glomeruli lacking capillary tufts [4]. Similarly, treatment of murine pups with mFlt(1-3)-IgG (a soluble VEGF receptor-1 chimeric protein) postnatally on day 1 and day 8, results in marked glomerular defects, including loss of endothelial cells, mesangial matrix accumulation and hypocellularity [10]. These results suggest that tight control of VEGF-A expression is required for normal glomerular development and well-being.

The close temporal and spatial association of VEGF-A expression (by podocytes) and its receptors (on glomerular endothelial cells) suggests that VEGF-A plays a pivotal role in the maintenance of glomerular integrity through the existence of a paracrine loop [11], and dysregulation of glomerular VEGF-A expression has been implicated in a wide range of renal diseases in humans [11]. Moreover, VEGF-A₁₆₅ acts as an autocrine growth factor on both proliferating and differentiating glomerular visceral epithelial cells (podocytes) [9], and that this results in prolonged survival and resistance to apoptosis, associated with changes in intracellular calcium concentration [12].

Isoforms of VEGF-A, termed according to their amino acid number, are generated by the differential splicing of eight exons of the full-length pre-mRNA from a single VEGF-A gene [WO-A-03/012105]. The differential splicing of exons 6 and 7 generates isoforms with differing heparin binding affinities [13], whilst the differential splicing of exon 8 (the terminal exon) generates two families of isoforms, pro-angiogenic and anti-angiogenic, that differ by only six amino acids at their C-terminus [14]. The pro-angiogenic VEGF-A isoforms, i.e. VEGF₁₂₁, VEGF₁₆₅ and VEGF₁₈₉ (collectively termed VEGFₓₓₓ, where xxx is the number of amino acids encoded) are formed by the selection of a proximal splice site in exon 8, termed exon 8a, which results in an open reading frame of 6 amino acids being translated. The anti-angiogenic VEGF-A isoforms are generated by the use of a more distal splice site in exon 8, termed exon 8b, resulting in an open reading frame of the same number of nucleotides as proximal (or pro-angiogenic) splice variants, but encoding a different amino acid sequence. Thus, the resulting proteins are of the same amino acid length as the conventional isoforms and are collectively termed VEGFₓₓₓb [15].

The first anti-angiogenic isoform to be identified from human renal cortex was VEGF₁₆₅b [14]. VEGF₁₆₅b inhibits VEGF₁₆₅ and hypoxia driven angiogenesis in vivo in rat, rabbit and mouse models of physiological and pathological angiogenesis [16] [17]. VEGF₁₆₅b does result in weak and tardy signalling through MAPK in microvascular endothelial cells in vitro [18] and induces a rapid but transient puff of fluid extravasation upon first exposure in intact microvessels in vivo but does not stimulate a sustained change in water permeability of microvessels [19]. VEGF₁₆₅b therefore does appear to have a stimulatory physiological role. VEGFₓₓₓb at the protein level appears to be the dominant isoform in many adult tissues, such as ocular tissues, colon and pancreatic islets [15]. VEGFₓₓₓb may therefore play a role in defining the physiological phenotype of the normal mature glomerulus (high permeability to water, low to protein in the absence of angiogenesis).

The Glomerular Filtration Barrier (GFB) is a unique multi-layered structure (41) demonstrating a striking dichotomy in its ability to restrict the extra-vasation of molecules of different sizes, shapes and charges. Poorly permeable to large, lipid insoluble or anionic molecules, the GFB is highly permeable to water, and small water-soluble agents. In glomerular disease this strict segregation is impaired or lost. In practice this is most frequently manifested by albumin in the urine. The mechanisms that underlie the phenomenon of proteinuria have been widely investigated, both because of its link to glomerular disease (heavy proteinuria tends to be associated with more severe glomerular lesions) and because, even at modest levels, proteinuria is now categorized as a major risk factor for vascular disease (8), even amongst the general population (19, 34). A detailed understanding of the factors that govern glomerular perm-selectivity, and the loss thereof in disease, is however still awaited. Although the controlling mechanisms of the normal glomerular phenotype are probably highly complex, in simple terms, they are likely to depend on two general factors: a) physical structure (eg foot processes, slit diaphragms and related proteins, fenestrae, GBM, glycocalyx, sub-podocyte space) and b) the function of cell types that contribute to the barrier, either through physical change (eg podocyte movement, contraction, effacement) or growth factor expression/secretion (eg VEGF-A, Angiopoietin-1, VEGF-C), i.e. podocyte-derived agents that are known to influence permeability in other micro-vascular beds and, the receptors for which, reside on the glomerular endothelial cells (15), and sometimes on the podocytes themselves (16)).

The specific role of podocyte-derived VEGF remains contentious, however its angiogenic/permeability potency ensures continuing interest, although in the context of a VEGF glomerular literature that is replete with apparent contradictions and, on initial inspection, unexplainable observations. For example:

i) transgenic podocytes-specific VEGF₁₆₄ over-expression in mice leads to proteinuria, collapsing nephropathy, uraemia and death at 5 days post birth (14), however podocytes VEGF-A glomerular reduction (heterozygous inactivation) similarly demonstrates nephrotic syndrome, uraemia and death at 2-5 weeks, although in the context of glomerular endotheliosis (14);

ii) in mature glomeruli, induced transgenic podocyte specific VEGF₁₆₄ over-expression results in proteinuria within hours of genetic stimulation (Quaggin personal communication), however systemic inhibition of VEGF with Avastin in humans also causes proteinuria (49) and occasionally renal failure (13);

iii) anti-VEGF antibody administration reduces proteinuria in an animal model of diabetic nephropathy (9) but induces proteinuria in normal animals (45) and VEGF administration in some non-diabetic animal models ameliorates glomerular injury (32).

Many of these carefully conducted studies are irreconcilable if VEGF is only regarded as a pro-angiogenic, pro-permeability vasodilator acting solely on endothelial cells.

Two key changes in our understanding of VEGF have recently forced a radical re-evaluation of VEGF biology.

The first is the identification of the anti-angiogenic VEGFₓₓₓb family of peptides in 2002 (1). In essence VEGF-A is 2 peptide families derived by alternative mRNA splicing from an 8 exon gene on chromosome 6. Family members, numbered by their amino acid content, have strikingly contrasting properties (22). The distinguishing content of the families is the 18 nucleotide open reading frame encoded by the final exon, coding for 6 amino acids—the inclusion of exon 8a resulting in the conventional pro-angiogenic, pro-permeability family of peptides (VEGFₓₓₓ). Replacement of 8a by exon 8b in the VEGFₓₓₓb family significantly influences the properties of these products in vivo, producing peptides that are anti-angiogenic and inhibit rather than promote tumour growth (1, 35, 37, 46, 48). Alternative splicing of exons 6 and 7 produce multiple isoforms within each family with differing heparin binding properties. The dominant member in each family contains 165 amino acids (see FIG. 8 of the accompanying drawings)

The second change in VEGF biology has been that, despite its nomenclature, VEGF is not endothelial cell specific, enhancing the survival of non-endothelial cells. VEGF₁₆₅, for example, is neuroprotective (26) and both VEGF₁₆₅ and VEGF₁₆₅b have human podocyte cyto-protective properties (16, 17). The VEGFₓₓₓb family, for example VEGF₁₆₅b, has been shown by the test data below to demonstrate human podocyte cyto-protective properties.

In most studies of VEGF-A in developing or mature glomerulus, a role of VEGF₁₆₅, or of other pro-angiogenic splice variants, has been investigated or assumed. Previous studies have used antibodies that detect both families of VEGF-A isoforms (pan-VEGF antibodies) as there were no antibodies or probes that distinguished between the VEGFₓₓₓ and VEGFₓₓₓb families of isoforms.

Schumacher et al [20] demonstrated that VEGF₁₆₅b is expressed at a greater level than VEGF₁₆₅ in glomeruli of the healthy adult human kidney, whereas VEGF₁₆₅b is absent from glomeruli of neonatal human kidneys in the fatal neonatal condition Denys-Drash syndrome. However, Denys-Drash syndrome is known to be a congenital renal abnormality caused by a mutation of the WT1 gene. Therefore, no predictions concerning any utility of VEGFₓₓₓb in treatment of microvascular hyperpermeability disorders, or any role of VEGFₓₓₓb isoform expression in normal renal development and function can be extrapolated from Schumacher's finding.

The present invention is based on our unanticipated finding that VEGFₓₓₓb is active against a range of microvascular hyperpermeability disorders, disorders of regulation of the pro-angiogenic pro-permeability properties of VEGFₓₓₓ isoforms and disorders of epithelial cell survival and permeability, particularly renal hyperpermeability disorders relating to the GFB and particularly VEGF-dependent disorders of these types.

In particular, the in vitro data below show that VEGF₁₆₅b reduces VEGF₁₆₅-induced human endothelial monolayer permeability, in addition to being anti-angiogenic in vivo. The in vivo data for heterozygous and homozygous transgenic animals that constitutively over-express VEGF₁₆₅b in podocytes under the control of the nephrin promoter show that sustained expression of exon 8b-containing VEGF peptides will produce different whole animal and organ phenotype from transgenic animals over-expressing exon 8a containing peptides. The homozygous transgenic animals have a lowered urinary protein:creatinine ratio and a significantly reduced single glomerular normalised ultra-filtration fraction (LpA/Vi), accompanied by a reduced endothelial fenestral density. In addition, VEGF₁₆₅b over-expression significantly reduced endogenous expression of murine total VEGF. The fenestration number and/or size of the filtration membrane of the animals' podocytes is markedly reduced by VEGFₓₓₓb, suggesting this as a part of the mechanism for the reduced microvascular permeability and that the same phenomenon would be expected in other epithelial filtration membranes. This mouse model data is thus predictive of activity of VEGFₓₓₓb, for example VEGF₁₆₅b, against in vivo vascular hyperpermeability disorders, or for regulating the pro-angiogenic pro-permeability properties of VEGFₓₓₓ isoforms, or for supporting epithelial cell survival without increased permeability, or for reducing the nature (for example the number density and/or size) of fenestrations of epithelial filtration membranes in vivo.

However, the action of VEGF is not limited to epithelial cells of the kidney. Retinal epithelial and endothelial cell loss are key events during progression of a number of ocular pathologies. For instance, diabetic retinopathy (DR) is associated with vascular closure, and subsequent ischemia, followed by hypoxia induced proliferative angiogenesis. In advanced retinal neovascularisation (RNV) vitreous haemorrhage, fibrosis and retinal detachment may occur. Severe DR is the most common reason for registration of blindness in the working population of developed countries despite conventional treatments. Additionally, retinal pigment epithelial cell loss in age related macular degeneration can contribute to geographic atrophy, and possibly invasive choroidal angiogenesis seen in neovascular AMD.

It is increasingly clear that inhibition of angiogenesis prevents ocular neovascularization in humans, and can prevent progression in models of proliferative RNV, which occurs through hypoxia driven expression of angiogenic vascular endothelial growth factor (VEGF) and choroidal neovascularisation (CNV) resulting from metabolic insult to RPE cells, possibly involving excess oxidised cholesterol uptake. Inhibitors of VEGF have been shown to be effective in treating the choroidal neovascularisation seen in age-related macular degeneration by inhibiting angiogenesis and reducing vascular permeability. They have also been shown to induce endothelial cell death and vascular regression. These latter properties are undesirable in the hypoxic diabetic eye so their use as a treatment for proliferative diabetic retinopathy is limited.

Inhibitory splice variants of VEGF-A—VEGFₓₓₓb—block the ability of VEGF to stimulate endothelial proliferation and migration, vasodilatation and tube formation in vitro. VEGF-A₁₆₅b and VEGF₁₂₁b have also been shown to inhibit angiogenesis in rabbit cornea, mouse mammary gland and skin, rat mesentery, chick chorioallantoic membrane and in five different tumour models. The presence of both angiogenic and anti-angiogenic isoforms in human retina, vitreous and iris has been demonstrated, as well as in the rodent eye. Furthermore it has been shown that whilst inhibitory VEGFₓₓₓb isoforms are the most abundant species in normal vitreous, they are relatively downregulated in diabetic vitreous resulting in a switch to an angiogenic phenotype. Moreover, the pro-angiogenic isoform VEGF-A₁₆₅ has been shown to act as a neuroprotective agent during retinal ischemia. There appears, therefore, to be a contradiction, in that endogenously, the eye has high levels of VEGF-A₁₆₅b, which is a competitive inhibitor of the actions of VEGF-A₁₆₅ in normal physiology, and yet it is well vascularised, and has healthy neurons. It is conceivable therefore that the VEGF-A₁₆₅b mediated inhibition of angiogenesis in the eye does not result in vascular regression, endothelial cell death, or neuronal impairment. It may specifically target VEGF-A₁₆₅ mediated neovascularisation, i.e. the formation of additional new vessels in the retina, rather than re-vascularisation—the reformation of existing blood vessels back into previously vascularised areas of the retina. If VEGF-A₁₆₅b is cytoprotective for epithelial cells of the human glomerulus, it may be similarly cytoprotective for retinal epithelial and endothelial cells. The effect of VEGF-A₁₆₅b on endothelial and retinal epithelial survival neovascularisation, and revascularisation has been investigated and the results are set forth below.

### SUMMARY

According to a first aspect of the present invention, there is provided VEGFₓₓₓb active agent for use in treating or preventing microvascular hyperpermeability disorders, or in regulating the pro-angiogenic pro-permeability properties of VEGFₓₓₓ isoforms, or in supporting epithelial cell survival with or without increased vascular permeability, or in reducing the nature (for example the number density and/or size) of fenestrations of epithelial filtration membranes.

According to a second aspect of the present invention, there is provided a method of treating or preventing microvascular hyperpermeability disorders, or in regulating the pro-angiogenic pro-permeability properties of VEGFₓₓₓ isoforms, or in supporting epithelial cell survival with or without increased vascular permeability, or in reducing the nature (for example the number density and/or size) of fenestrations of epithelial filtration membranes, which comprises administering to a subject having or susceptible to such a disorder, an effective amount of a VEGFₓₓₓb active agent.

According to a third aspect of the present invention, there is provided the use of a VEGFₓₓₓb active agent in the manufacture of a composition (e.g. a pharmaceutical composition) for treating or preventing microvascular hyperpermeability disorders, or in regulating the pro-angiogenic pro-permeability properties of VEGFₓₓₓ isoforms, or in supporting epithelial cell survival with or without increased vascular permeability, or in reducing the nature (for example the number density and/or size) of fenestrations of epithelial filtration membranes.

According to a fourth aspect of the present invention, there is provided a method of reducing the permeability of a microvascular membrane in vivo or in vitro (including ex vivo), or regulating the pro-angiogenic pro-permeability properties of VEGFₓₓₓ isoforms, or supporting epithelial cell survival with or without increased vascular permeability, or reducing the nature (for example the number density and/or size) of fenestrations of epithelial filtration membranes in vivo, which comprises contacting the membrane with an effective amount of a VEGFₓₓₓb active agent.

According to a fifth aspect of the present invention, there is provided a VEGFₓₓₓb active agent for use in treating or preventing disorders resulting from increased epithelial cell degeneration or decreased epithelial survival. The effect of the use of said agent is that epithelial cell survival is supported, or epithelial cell death is prevented.

According to a sixth aspect of the present invention, there is provided a method of treating or preventing disorders resulting from increased epithelial cell degeneration or decreased epithelial survival, which comprises administering to a subject having or susceptible to such a disorder, an effective amount of a VEGFₓₓₓb active agent.

According to a seventh aspect of the present invention, there is provided the use of a VEGFₓₓₓb active agent in the manufacture of a composition (e.g. a pharmaceutical composition) for treating or preventing disorders resulting from increased epithelial cell degeneration or decreased epithelial survival.

According to an eighth aspect of the present invention, there is provided a method treating or preventing disorders resulting from increased epithelial cell degeneration or decreased epithelial survival, which comprises contacting the membrane with an effective amount of a VEGFₓₓₓb active agent.

In the fifth to eighth aspects of the invention, and corresponding similar aspects below, the effect of the use of said agent is that epithelial cell survival is supported, or epithelial cell death is prevented. Support of epithelial cell survival may be associated with or without increased vascular permeability of the epithelial membrane.

The present invention also includes the corresponding use—in place of or additional to the VEGFₓₓₓb—of an agent, such as those described and claimed in WO-A-2008/110777, which selectively promotes the presence or expression of VEGFₓₓₓb, relative to a normal or untreated subject or in preference (i.e. relative) to VEGFₓₓₓ in cells of a subject or in vitro. The use of such an agent constitutes a further aspect of the present invention. In particular, there may be mentioned agents that favour distal splice site (DSS) splicing during processing of VEGF pre-mRNA transcribed from the C terminal exon 8 of the VEGF-A gene. Such agents may, if desired be used in association with one or more controlling agents for the splicing which suppresses or inhibits proximal splice site (PSS) splicing during processing of VEGF pre-mRNA transcribed from the C terminal exon 8 of the VEGF-A gene (see WO-A-2008/110777). Agents which selectively inhibit the function of VEGFₓₓₓ, for example specific anti-VEGFₓₓₓ antibodies, are further examples of agents which selectively promote the presence of VEGFₓₓₓb relative to VEGFₓₓₓ in cells of a subject or in vitro.

The VEGFₓₓₓb may be full VEGFₓₓₓb protein or an anti-angiogenic fragment thereof, or other VEGFₓₓₓb derived or related protein material which is functionally equivalent to full VEGFₓₓₓb protein in relevant respects. The term “VEGFₓₓₓb” is to be understood in this manner.

The terms “active agent” and “VEGFₓₓₓb active agent” used herein encompass VEGFₓₓₓb protein material and agents which promote the presence or endogenous expression of VEGFₓₓₓb relative to the normal or untreated subject, or in preference (i.e. relative) to VEGFₓₓₓ, in vivo or in vitro.

According to a further aspect of the present invention, there is provided a method of testing a subject for risk or susceptibility to microvascular hyperpermeability disorders, disorders of regulation of the pro-angiogenic pro-permeability properties of VEGFₓₓₓ isoforms, disorders of epithelial cell survival and permeability, and/or disorders in the nature (for example the number density and/or size) of fenestrations of epithelial filtration membranes, the method comprising obtaining a biological sample from the subject, and assaying the levels of VEGFₓₓₓb in the sample relative to normal absolute VEGFₓₓₓb levels or relative to normal VEGFₓₓₓb : VEGFₓₓₓ ratio. Depending on the results of the assay, the method according to the second aspect of the present invention may be applied to the subject.

According to a further aspect of the present invention, there is provided a method of testing a subject for risk or susceptibility to microvascular hyperpermeability disorders, disorders of regulation of the pro-angiogenic pro-permeability properties of VEGFₓₓₓ isoforms, disorders of epithelial cell survival and permeability, and/or disorders in the nature (for example the number density and/or size) of fenestrations of epithelial filtration membranes, the method comprising genotyping the subject to determine a risk of underexpressing VEGFₓₓₓb relative to normal absolute VEGFₓₓₓb level or relative to normal VEGFₓₓₓb : VEGFₓₓₓ ratio. Depending on the results of the assay, the method according to the second aspect of the present invention may be applied to the subject.

According to a further aspect of the present invention, there is provided a method of testing a subject for risk or susceptibility to disorders of epithelial cell survival, the method comprising obtaining a biological sample from the subject, and assaying the levels of VEGFₓₓₓb in the sample relative to normal absolute VEGFₓₓₓb levels or relative to normal VEGFₓₓₓb : VEGFₓₓₓ ratio. Depending on the results of the assay, the method according to the second aspect of the present invention may be applied to the subject.

According to a further aspect of the present invention, there is provided a method of testing a subject for risk or susceptibility to disorders of epithelial cell survival, the method comprising genotyping the subject to determine a risk of underexpressing VEGFₓₓₓb relative to normal absolute VEGFₓₓₓb level or relative to normal VEGFₓₓₓb: VEGFₓₓₓ ratio. Depending on the results of the assay, the method according to the second aspect of the present invention may be applied to the subject.

### DETAILED DESCRIPTION

VEGFₓₓₓb Active Agent

The term “VEGFₓₓₓb active agent” encompasses VEGFₓₓₓb protein materials (including, but not limited to, full protein and anti-angiogenic fragments thereof) and agents which promote the presence or endogenous expression of VEGFₓₓₓb relative to the normal or untreated subject, or in preference (i.e. relative) to VEGFₓₓₓ, in vivo or in vitro.

The VEGFₓₓₓb active agent used in the present invention may be prepared by any suitable means.

The use of agents, acting on cells to promote the endogenous expression of VEGFₓₓₓb in preference (i.e. relative) to VEGFₓₓₓ in the cells, is one possible way of preparing the VEGFₓₓₓb for use in the present invention. For further details of the agents, see WO-A-2008/110777.

The term “VEGFₓₓₓb active agent” thus includes within its scope an expression vector system which causes the expression of the VEGFₓₓₓb in a host organism. This may be the subject to be treated or another organism suitable to the subject to be treated. Such an expression vector system suitably comprises a promoter nucleotide sequence operably associated a nucleotide sequence coding for the VEGFₓₓₓb, whereby the VEGFₓₓₓb can be expressed in the host organism under suitable conditions of transfection and incubation. Further details are provided below in the section headed “Gene Therapy”.

The term “VEGFₓₓₓb active agent” thus also includes within its scope an inhibition system for VEGFₓₓₓ in a host organism, suitably the subject to be treated, whereby the proportion of active VEGFₓₓₓb to VEGFₓₓₓ is increased in the host organism or particular tissues thereof. Such an inhibition system may, for example, comprise a specific anti-VEGFₓₓₓ antibody, for example a monoclonal or polyclonal specific anti-VEGFₓₓₓ antibody [15, 16, 25]. The inhibition system may alternatively comprise an expression vector system which causes the expression of an inhibition system for VEGFₓₓₓ in a host organism. Such an expression vector system suitably comprises a promoter nucleotide sequence operably associated a nucleotide sequence coding for a protein inhibition system for VEGFₓₓₓ, such as a specific anti-VEGFₓₓₓ antibody, whereby the protein inhibition system for VEGFₓₓₓ can be expressed in the host organism under suitable conditions of transfection and incubation.

More than one type of VEGFₓₓₓb active agent, and/or more than one embodiment of any particular type of VEGFₓₓₓb active agent, may be used simultaneously or sequentially if desired.

The VEGFₓₓₓb may for example, comprise one or more of VEGF₁₆₅b, VEGF₁₈₉b, VEGF₁₄₅b, VEGF₁₈₃b and VEGF₁₂₁b. The VEGFₓₓₓb suitably comprises recombinant VEGFₓₓₓb, preferably recombinant human VEGFₓₓₓb (rhVEGFₓₓₓb).

The VEGFₓₓₓb preferably comprises VEGF₁₆₅b, e.g. recombinant VEGF₁₆₅b, such as recombinant human VEGF₁₆₅b (rhVEGF₁₆₅b).

The VEGFₓₓₓb may, for example, consist essentially of VEGF₁₆₅b, e.g. recombinant VEGF₁₆₅b, such as recombinant human VEGF₁₆₅b (rhVEGF₁₆₅b). The VEGFₓₓₓb may, for example, consist of VEGF₁₆₅b, e.g. recombinant VEGF₁₆₅b, such as recombinant human VEGF₁₆₅b (rhVEGF₁₆₅b).

VEGFₓₓₓb Active Agents which Selectively Promote the Expression of VEGFₓₓₓb in preference (i.e. Relative) to VEGFₓₓₓ in Cells of a Subject or in vitro

Such agents are described in the passage from page 6, line 22 to page 8, line 9 of WO-A-2008/110777, and elaborated in the remainder of WO-A-2008/110777 to the extent that favouring of DSS splicing over PSS splicing is concerned. Please refer to these passages of WO-A-2008/110777 for the discussion.

In particular, there may be mentioned Transforming Growth Factor (TGF)-b1, TGF-b R1, SRPK1 specific inhibitors (for example, SRPIN 340), T-cell intercellular antigen-1 (TIA-1), MKK3/MKK6-activatable MAP kinases (for example, p38 MAPK), Cdc20-like (Clk) family kinases Clk1/sty, Clk2, Clk3 and Clk4, the SR splicing factor SRp55, their in vivo activators, upregulators and potentiators, functionally active analogues and functionally active fragments of any of the foregoing, modified forms of any of the foregoing having a secondary functionality useful for control of their primary activity or the effects thereof, expression vector systems for expressing any of the foregoing agents in vivo, transcription/translation blocking agents which bind to the PSS of exon 8a of the pre-mRNA and/or at the region of the pre-mRNA to which a splicing regulatory protein binds, to inhibit proximal splicing (for example, morpholinos or other synthetic blocking agents, peptide conjugates, RNA binding proteins, RNA interference (RNAi) poly- and oligonucleotide blocking agents (for example dsRNA, microRNA (miRNA), siRNA), peptide nucleic acid (PNA), SR protein kinase (SRPK) inhibitors (for example, SRPIN340) and other mechanistically analogous SRPK inhibitors, particularly inhibitors which bind at the SRPK catalytic domain), or any combination thereof.

Such an expression vector system suitably comprises a promoter nucleotide sequence operably associated a nucleotide sequence coding for the agent promoting expression of VEGFₓₓₓb in preference to VEGFₓₓₓ, whereby the agent promoting expression of VEGFₓₓₓb in preference to VEGFₓₓₓ can be expressed in a host organism, suitably the subject to be treated, under suitable conditions of transfection and incubation. Further details are provided below in the section headed “Gene Therapy”.

Conditions and Disorders to be Treated

Microvascular hyperpermeability, disorders of regulation of the pro-angiogenic pro-permeability properties of VEGFₓₓₓ isoforms, disorders of epithelial cell survival and permeability, and/or disorders in the nature (for example the number density and/or size) of fenestrations of epithelial filtration membranes underlie a number of serious medical conditions.

Examples of such conditions include, for example, proteinuria, uraemia, microalbuminuria, hypoalbuminemia, renal hyperfiltration, nephrotic syndrome, renal failure, pulmonary hypertension, capillary hyperpermeability, microaneurysms, oedema and vascular complications of diabetes.

Examples of such vascular complications of diabetes include, for example, diabetic retinopathy, both proliferative and non-proliferative, and diabetic nephropathy. Vascular complications of diabetes can be associated with either Type I or Type II diabetes.

The loss of proteins from the blood can lead to further complications, for example thromboses, especially thromboses in the brain, and susceptibility to infections. Loss of natural proteins from the blood can seriously impair the efficacy of cancer therapies.

The microvascular hyperpermeability disorder may particularly be a renal disorder, for example a permeability disorder of the GFB, for example a permeability disorder of the podocytes.

Examples of disorders where treatment to support epithelial cell survival would be effective are as follows: acute pulmonary fibrotic disease, adult respiratory distress syndrome, adult respiratory distress syndrome, advanced cancer, allergic respiratory disease, alveolar injury, angiogenesis, arthritis, ascites, asthma, asthma or edema following burns, atherosclerosis, autoimmune diseases, bone resorption, bullous disorder associated with subepidermal blister formation including bullous pemphigoid, cardiovascular condition, certain kidney diseases associated with proliferation of glomerular or mesangial cells, chronic and allergic inflammation, chronic lung disease, chronic occlusive pulmonary disease, cirrhosis, corneal angiogenisis, corneal disease, coronary and cerebral collateral vascularization, coronary restenosis, damage following heart disease, dermatitis herpetiformis, diabetes, diabetic nephropathy, diabetic retinopathy, endotoxic shock, erythema multiforme, fibrosis, glomerular nephritis, glomerulonophritis, graft rejection, gram negative sepsis, hemangioma, hepatic cirrhosis, hepatic failure, Herpes Zoster, host-versus-graft reaction (ischemia reperfusion injury and allograft rejections of kidney, liver, heart, and skin), impaired wound healing in infection, infection by Herpes simplex, infection from human immunodeficiency virus (HIV), inflammation, cancer, inflammatory bowel disease (Crohn's disease and ulcerative colitis), inflammatory conditions, in-stent restenosis, in-stent stenosis, ischemia, ischemic retinal-vein occlusion, ischemic retinopathy, Kaposi's sarcoma, keloid, liver disease during acute inflammation, lung allograft rejection (obliterative bronchitis), lymphoid malignancy, macular degeneration retinopathy of prematurity, myelodysplastic syndromes, myocardial angiogenesis, neovascular glaucoma, non-insulin-dependent diabetes mellitus (NIDDM), obliterative bronchiolitis, ocular conditions or diseases, ocular diseases associated with retinal vessel proliferation, Osier-Weber-Rendu disease, osteoarthritis, ovarian hyperstimulation syndrome, Paget's disease, pancreatitis, pemphigoid, polycystic kidney disease, polyps, postmenopausal osteoperosis, preeclampsia, psoriasis, pulmonary edema, pulmonary fibrosis, pulmonary sarcoidosis, restenosis, restenosis, retinopathy including diabetic retinopathy, retinopathy of prematurity and age related macular degeneration; rheumatoid arthritis, rheumatoid arthritis, rubeosis, sarcoidosis, sepsis, stroke, synovitis, systemic lupus erythematosus, throiditis, thrombic micoangiopathy syndromes, transplant rejection, trauma, tumor-associated angiogenesis, vascular graft restenosis, vascular graft restenosis, von Hippel Lindau disease, wound healing.

In particular patients, the disorder of epithelial cell survival (e.g. epithelial cell loss or degeneration, decreased epithelial cell survival, and disorders characterised by these conditions) may be independent of any associated hyperpermeability, or any hyperpermeability may be secondary to epithelial cell loss. Examples of such disorders are dry age related macular degeneration; diabetic and non-diabetic nephropathy without proteinuria; glumerulosclerosis; lung diseases characterised primarily by epithelial cell loss with no or secondary hyperpermeability response such as chronic obstructive airway disease, pulmonary fibrosis and asthma; alpha-1 anti-trypsin deficiency; inflammatory bowel disease; inflammatory arthritis; and primary biliary cirrhosis.

The use of VEGFₓₓₓb agents to treat epithelial cell loss or degeneration, decreased epithelial cell survival, and disorders thereof is surprising in view of the fact that such conditions and disorders are often associated with hyperpermeability. The prior knowledge that VEGF₁₆₅b increased permeability although fleetingly (a few seconds) but to a greater degree than VEGF₁₆₅ (Reference [19], (20)) suggests that VEGFₓₓₓb agents would be contraindicated in hyperpermeable states. However, the present invention shows that chronic hyperpermeability is in fact inhibited by VEGFₓₓₓb agents. From this finding the present invention enables patients with epithelial cell disorders having potential secondary or associated hyperpermeability complications to be treated safely with VEGFₓₓₓb agents.

Similarly, the use of VEGFₓₓₓb agents to treat hyperpermeability and hyperpermeability disorders in patients having disorders where loss of epithelial cells or epithelial cell function would be dangerous to life is a specific aspect of the present invention. Such patients include those with the hyperpermeability conditions mentioned above, for example diabetic and non-diabetic nephropathy. Prior to the present invention, it was not appreciated that such patients could be treated using VEGFₓₓₓb agents without risk of adverse side effects on their epithelial cells or epithelial cell function. The present invention enables such patients to be treated safely with VEGFₓₓₓb agents.

The present invention may be used in the treatment of macular dystrophy. This includes: Stargardt disease/fundus flavimaculatus; Stargardt-like macular dystrophy; Stargardt-like macular dystrophy; Autosomal dominant “bull' seye” macular dystrophy Best macular dystrophy; Adult vitelliform dystrophy; Pattern dystrophy; Doyne honeycomb retinal dystrophy; North Carolina macular dystrophy; Autosomal dominant macular dystrophy resembling MCDR1; North Carolina-like macular dystrophy associated with deafness; Progressive bifocal chorioretinal atrophy; Sorsby's fundus dystrophy; Central areolar choroidal dystrophy; Dominant cystoid macular dystrophy; Juvenile retinoschisis; Occult Macular Dystrophy; Non-familial Occult Macular Dystrophy.

The disorder may particularly be a disorder of the retinal epithelium, such as geographic atrophy, age related macular degeneration.

The VEGFₓₓₓb active agent may, if desired, be co-administered with one or more additional active agent, for example one or more agent selected from, but not limited to, anti-angiogenic compounds, namely a compound capable of inhibiting the formation of blood vessels. Suitable compounds include, for example, one or more ACE (angiotensin converting enzyme) inhibitors, one or more angiotensin II receptor antagonists, one or more corticosteroids, or any combination thereof.

Testing for Disorders

According to the present invention, a biological sample taken from subject can be tested for risk or susceptibility to microvascular hyperpermeability disorders, disorders of regulation of the pro-angiogenic pro-permeability properties of VEGFₓₓₓ isoforms, disorders of epithelial cell survival and permeability, and/or disorders in the nature (for example the number density and/or size) of fenestrations of epithelial filtration membranes. The method comprises assaying the levels of VEGFₓₓₓb or the relative levels of VEGFₓₓₓb and VEGFₓₓₓ in the sample, or genotyping the subject, using the material from the biological sample, to determine a risk of underexpressing VEGFₓₓₓb relative to normal absolute VEGFₓₓₓb level or relative to normal VEGFₓₓₓb : VEGFₓₓₓ ratio.

The sample is preferably a body fluid sample such as urine, blood, blood plasma, saliva or serum.

A level of VEGFₓₓₓb or a relative level of VEGFₓₓₓb to VEGFₓₓₓ in the sample which is below normal levels is generally correlated to an increased risk or susceptibility to one or more of the disorders, for example one or more of the specific diseases or disorders that may be treated according to the present invention.

The levels of VEGFₓₓₓb or the relative levels of VEGFₓₓₓb and VEGFₓₓₓ in the sample are assayed in ways well established in the art, referred to in the references cited herein and in the following Examples, and a detailed discussion is not required. The risk or susceptibility is determined according to comparison data, obtained from groups of normal and diseased subjects, which correlates the levels to the risk or susceptibility.

The above also applies in the case where the biological sample taken from subject is tested for risk or susceptibility to disorders of epithelial cell survival.

Compositions and Administration

The active agent may be administered in the form of a composition comprising the active agent and any suitable additional component. The composition may, for example, be a pharmaceutical composition (medicament).

According to a further aspect of the present invention, there is provided a composition comprising an effective amount of VEGFₓₓₓb active agent for use in treating or preventing microvascular hyperpermeability disorders, or in regulating the pro-angiogenic pro-permeability properties of VEGFₓₓₓ isoforms, or in supporting epithelial cell survival without increased permeability, or in reducing the nature (for example the number density and/or size) of fenestrations of epithelial filtration membranes in vivo or in vitro (including ex vivo).

According to a further aspect of the present invention, there is provided a composition comprising an effective amount of VEGFₓₓₓb active agent for use in supporting epithelial cell survival.

The active agent according to the present invention may be administered in the form of a composition comprising the active agent and any suitable additional component. The composition may, for example, be a pharmaceutical composition (medicament), suitably for parenteral administration (e.g. injection, implantation or infusion).

The term “pharmaceutical composition” or “medicament” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations. Techniques and formulations generally may be found in Remington, The Science and Practice of Pharmacy, Mack Publishing Co., Easton, Pa., latest edition.

Liquid form preparations include solutions, suspensions, and emulsions. As an example may be mentioned water or water-propylene glycol solutions for parenteral injection. Liquid preparations can also be formulated in solution in aqueous polyethylene glycol solution.

Also included are solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container or apparatus. The solid form preparations intended to be converted to liquid form may contain, in addition to the active material, flavourings, colourants, stabilizers, buffers, artificial and natural sweeteners, dispersants, thickeners, solubilising agents, and the like. The liquid utilized for preparing the liquid form preparation may be water, isotonic water, ethanol, glycerine, propylene glycol, and the like as well as mixtures thereof. Naturally, the liquid utilized will be chosen with regard to the route of administration, for example, liquid preparations containing large amounts of ethanol are not suitable for parenteral use.

The dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

Gene Therapy

The present invention may alternatively be practiced using gene therapy. Gene therapy techniques are generally known in this art, and the present invention may suitably be put into practice in these generally known ways. The following discussion provides further outline explanation.

The gene therapies are broadly classified into two categories, i.e., in vivo and in vitro therapies. The in vivo gene therapy comprises introducing a therapeutic gene directly into the body, and the in vitro gene therapy comprises culturing a target cell in vitro, introducing a gene into the cell, and then, introducing the genetically modified cell into the body.

The gene transfer technologies are broadly divided into a viral vector-based transfer method using virus as a carrier, a non-viral delivery method using synthetic phospholipid or synthetic cationic polymer, and a physical method, such as electroporation or introducing a gene by applying temporary electrical stimulation to a cell membrane.

Among the gene transfer technologies, the viral vector-based transfer method is considered to be preferable for the gene therapy because the transfer of a genetic factor can be efficiently made with a vector with the loss of a portion or whole of replicative ability, which has a gene substituted a therapeutic gene. Examples of virus used as the virus carrier or vector include RNA virus vectors (retrovirus vectors, lentivirus vector, etc.), and DNA virus vectors (adenovirus vectors, adeno-associated virus vectors, etc.). In addition, its examples include herpes simplex viral vectors, alpha viral vectors, etc. Among them, retrovirus and adenovirus vectors are particularly actively studied.

The characteristics of retrovirus acting to integrate into the genome of host cells are that it is harmless to the human body, but can inhibit the function of normal cells upon integration. Also, it infects various cells, proliferates fast, can receive about 1-7 kb of foreign genes, and is capable of producing replication-deficient virus. However, it has disadvantages in that it is hard to infect cells after mitosis, it is difficult to transfer a gene in vivo, and the somatic cell tissue is needed to proliferate always in vitro. In addition, since it can be integrated into a proto-oncogene, it has the risk of mutation and can cause cell necrosis.

Meanwhile, adenovirus has various advantages for use as a cloning vector; it has moderate size, can be replicated within a cell nucleus, and is clinically nontoxic. Also, it is stable even when inserted with a foreign gene, and does not cause the rearrangement or loss of genes, can transform eucaryotes, and is stably expressed at a high level even when it is integrated into the chromosome of host cells. Good host cells for adenovirus are cells of causing human hematosis, lymphoma and myeloma. However, these cells are difficult to proliferate because they are linear DNAs. Also, it is not easy infected virus to be recovered, and they have low virus infection rate. Also, the expression of a transferred gene is the highest after 1-2 weeks, and in some cells, the expression is kept only for about 3-4 weeks. In addition, these have the problem of high immune antigenicity.

Adeno-associated virus (AAV) can overcome the above-described problems and at the same time, has many advantages for use as a gene therapeutic agent and thus is recently considered to be preferable. AAV, which is single-strand provirus, requires an assistant virus for replication, and the AAV genome is 4,680 by in size and can be inserted into any site of chromosome 19 of infected cells. A trans-gene is inserted into plasmid DNA linked with 145 by of each of two inverted terminal repeat sequence (ITR) and a signal sequence. This gene is transfected with another plasmid DNA expressing AAV rep and cap genes, and adenovirus is added as an assistant virus. AAV has advantages in that the range of its host cells to be transferred with a gene is wide, immune side effects due to repeated administration are little, and the gene expression time is long. Furthermore, it is stable even when the AAV genome is integrated into the chromosome of a host cell, and it does not cause the modification or rearrangement of gene expression in host cells. Since an AAV vector containing a CFTR gene was approved by NIH for the treatment of cystic fibrosis in 1994, it has been used for the clinical treatment of various diseases. An AAV vector containing a factor IX gene, which is a blood coagulation factor, is used for the treatment of hemophilia B, and the development of a therapeutic agent for hemophilia A with the AAV vector is currently being conducted. Also, AAV vectors containing various kinds of anticancer genes were certified for use as tumor vaccines.

Gene therapy, which is a method of treating diseases by gene transfer and expression, is used to adjust a certain gene, unlike the drug therapy. The ultimate purpose of the gene therapy is to obtain useful therapeutic effects by genetically modifying a living gene. The gene therapy has various advantages, such as the accurate transfer of a genetic factor into a disease site, the complete decomposition of the genetic factor in vivo, the absence of toxicity and immune antigenicity, and the long-term stable expression of the genetic factor and thus is spotlighted in connection with the present invention as a potentially suitable route of treatment.

The host cell for the gene therapy, to which the gene therapy is targeted, is preferably a podocyte.

In general, reference herein to the presence of one of a specified group of compounds, for example VEGFₓₓₓb, includes within its scope the presence of a mixture of two or more of such compounds.

“Treating or Preventing”

The expression “treating or preventing” and analogous terms used herein refers to all forms of healthcare intended to remove or avoid the disorder or to relieve its symptoms, including preventive, curative and palliative care, as judged according to any of the tests available according to the prevailing medical practice. An intervention that aims with reasonable expectation to achieve a particular result but does not always do so is included within the expression “treating or preventing”. An intervention that succeeds in slowing or halting progression of a disorder is included within the expression “treating or preventing”.

“Susceptible to”

The expression “susceptible to” and analogous terms used herein refers particularly to individuals at a higher than normal risk of developing a medical disorder, or a personality change, as assessed using the known risk factors for the individual or disorder. Such individuals may, for example, be categorised as having a substantial risk of developing one or more particular disorders, to the extent that medication would be prescribed and/or special dietary, lifestyle or similar recommendations would be made to that individual.

Subject

The subject is preferably a human or non-human mammal.

Besides being useful for human treatment, the present invention is also useful in a range of mammals. Such mammals include non-human primates (e.g. apes, monkeys and lemurs), for example in zoos, companion animals such as cats or dogs, working and sporting animals such as dogs, horses and ponies, farm animals, for example pigs, sheep, goats, deer, oxen and cattle, and laboratory animals such as rodents (e.g. rabbits, rats, mice, hamsters, gerbils or guinea pigs).

Where the disorder or function to be treated is exclusive to humans, then it will be understood that the mammal to be treated is a human. The same applies respectively to any other mammalian species if the disorder or function to be treated is exclusive to that species.

Where the context allows, the subject may include an unborn fetus. In the assay and genotyping methods for testing a subject for risk or susceptibility to microvascular hyperpermeability disorders, disorders of regulation of the pro-angiogenic pro-permeability properties of VEGFₓₓₓ isoforms, disorders of epithelial cell survival and permeability, and/or disorders in the nature (for example the number density and/or size) of fenestrations of epithelial filtration membranes, for example, the subject may be an unborn fetus and the method may be performed on a biological sample of fetal material, placental material or amniotic fluid. In the assay and genotyping methods for testing a subject for risk or susceptibility to disorders of epithelial cell survival, for example, the subject may be an unborn fetus and the method may be performed on a biological sample of fetal material, placental material or amniotic fluid.

The expression “human or non-human mammal” covers human and non-human mammals at all stages of development and ageing, including embryo, fetus, neonate, child, adolescent, young adult, mature adult and in old age.

### BRIEF DESCRIPTION OF THE DRAWINGS

In order to illustrate the invention further by way of non-limiting example, reference will now be made to the accompanying drawings and to the Examples which follow.

In the drawings:

FIG. 1 illustrates the expression of VEGF and its two families of isoforms in normal human renal cortex; VEGFₓₓₓb isoforms comprise over 45% of total VEGF in adult human renal cortical tissues.

FIG. 2 illustrates immunohistochemical staining of VEGFₓₓₓb and pan-VEGF in the adult kidney; VEGFₓₓₓb staining in the podocytes of the glomerulus (A, arrows), proximal and distal tubules of the renal cortex (B) and in the ascending thick and thin loop of Henle in the renal medulla (C) was clearly seen. Sections treated with a pan-VEGF antibody (D-F) show a comparable staining pattern. Matched mouse IgG controls were negative (G-I). Scales bar=30 μm, except A insert=10 μm. (PT=proximal tubule, DT=distal tubule, TLH=thin loop of Henle, TkLH=thick loop of Henle, CD=collecting ducts, VR=Vasa Recta).

FIG. 3 illustrates an overview of VEGFₓₓₓb and pan-VEGF immunohistochemical staining of the metanephric kidney; VEGFₓₓₓb (A and B) and pan-VEGF (C and D) staining was observed in specific developmental regions of the metanephric kidney of 10 (A and C) and 12 (B and D) week fetuses. Matched mouse IgG controls were negative (E and F). Scale bar=600 μm.

FIG. 4 illustrates VEGFₓₓₓb and pan-VEGF immunohistochemical staining during the early stages of nephrogenesis; Intracellular VEGFₓₓₓb staining (A to D) and pan-VEGF staining (E to H) was observed during glomerular development. VEGFₓₓₓb (A) and pan-VEGF (E) staining was observed at the various stages of nephrogenesis, with a specific staining pattern. For example, in the condensed vesicle (B, F) the staining was more polarised as the mesenchymal cells gained epithelial characteristics. In comma (C, G) and S-shaped bodies (D, H), VEGFₓₓₓb (C, D) and pan-VEGF (G, H) staining was localised to the primitive epithelial cells, especially on the apical side, and to the glomerular cleft. Scale Bar=30 μm. (GC=glomerular cleft, CV=condensed vesicle, CSB=comma shaped body, SSB=S-shaped body, G=glomeruli, arrow=diffuse staining of the glomerular cleft).

FIG. 5 illustrates VEGFₓₓₓb immunohistochemical staining during glomerular maturation; Apical and basolateral VEGFₓₓₓb (A,C,E) and pan-VEGF (B,D,F) staining. The presumptive podocytes and the glomerular cleft show diffuse staining in the capillary loop stage (A, B). As development of the glomerulus progresses and the presumptive podocytes mature, the intensity of the VEGFₓₓₓb staining decreases (C), whereas pan-VEGF staining is still intense (D) and VEGFₓₓₓb becomes more specific to a subpopulation of mature podocytes (E). Pan-VEGF staining is more widespread in the podocytes at this stage (F). Scale bar=50 μm. (GC=glomerular cleft, MD=macula densa, arrow=parietal epithelial cells)

FIG. 6 illustrates immunohistochemical staining of VEGFₓₓₓb and pan-VEGF in the tubules of the developing renal cortex and medulla; VEGFₓₓₓb staining was seen in the more proximal and distal regions of the convoluted tubules (A and C). Strong periluminal staining was observed in the proximal tubules and collecting ducts. Similar immunohistochemical staining was observed for pan-VEGF (B and D). Scale bars=30 μm. (GC=glomerular cleft, MD=macula densa, PT=proximal tubule, DT=distal tubule, CD=collecting ducts, VR=Vasa Recta).

FIG. 7 illustrates VEGF₁₆₅b is cytoprotective for glomerular epithelial cells, and inhibits VEGF₁₆₅ mediated migration and increased permeability of endothelial cells. (A) VEGF₁₆₅b dose dependently inhibits endothelial cell migration induced by VEGF₁₆₅. p<0.001,ANOVA. (B) VEGF₁₆₅b dose dependently prevents cell death in primary cultured podocytes. p<0.001,ANOVA. (C) VEGF₁₆₅b inhibits apoptosis induced by serum starvation. Flow cytometry of Annexin V and propidium iodide stained human conditionally immortalised podocytes (hCIP) treated with (Ci) 48 hrs serum starvation (SS) or (C2) 1 nM VEGF₁₆₅b and SS. V=viable population, N=necrotic, A=apoptotic, LA=Late apoptotic. (D) VEGF₁₆₅b reduces permeability of glomerular monolayers. VEGF₁₆₅b increases glomerular trans-endothelial electrical resistance (TEER) in cultured monolayers, VEGF₁₆₅ reduces TEER (increases permeability), and VEGF₁₆₅b inhibits the VEGF₁₆₅ mediated reduction. ***=p<0.001, **=p<0.01, compared with control, ++=p<0.01 compared with both VEGF₁₆₅b and VEGF₁₆₅. One way ANOVA and Bonferroni post hoc analysis.

FIG. 8 illustrates VEGF-A sub-families. Nomenclature is based on amino acid size of final peptide. Two mRNA isoform families are generated with differing heparin binding regions (exons 6 and 7). The pro-angiogenic isoforms (VEGFxxx, left) are generated by proximal splice site (PSS) selection in exon 8 (ie 8a) and the anti-angiogenic (VEGFxxxb, right) family from exon 8 distal splice site choice (exon 8b) (DSS). Thus VEGF165, formed by PSS selection in exon 8, has VEGF₁₆₅b as its distal splice site (DSS) sister isoform—the DSS selected mRNA encoding a protein of exactly the same length as VEGF165. Exon 6a′ occurs in VEGF₁₈₃ as a result of a conserved alternative splicing donor site in exon 6a and is 18 bp shorter than full length exon 6a. VEGF₂₀₆b has not yet been identified. UTR, untranslated region.

FIG. 9 illustrates generation of pNeph-VEGF₁₆₅b heterozygous transgenic mice. VEGF₁₆₅b was cloned into an expression vector under the control of the Nephrin promoter (Transgenic Construct FIG. 9Ai). When transfected into human podocytes, VEGF₁₆₅b expression was significantly greater than control vector, or untransfected cells (FIG. 9Aii). FIG. 9B illustrates a PCR screen of pups born from injected embryos. FIG. 9C shows Southern blot, confirming single insertion into genomic DNA. The gDNA was digested with EcoR1 and a probe made from the cDNA used to generate the transgenic. There was a single EcoR1 site in the 5′end of the insert, so multiple insertions should generate multiple different sized bands in the transgene. There was only a single band in line 1, indicating a single insertion point (insertions into multiple sites would generate multiple bands). Lines 1 was used for establishment of the subsequent heterozygous and homozygous breeding olonies. FIG. 9D shows Ultra-filtration fraction from founder lines was not different from each other.

FIG. 10 illustrates that pNeph-VEGF₁₆₅b heterozygous and homozygous transgenic mice over-express VEGF₁₆₅b in the renal visceral glomerular epithelial cells (podocytes). VEGF₁₆₅b expression was determined in the renal glomerulus (FIG. 10Ai and 10Aii). Exon 8b specific RT-PCR of renal cortex for transgene p<0.05, chi squared test for trend. N=3 per group. FIG. 10B shows immunohistochemistry using anti-human VEGF (A20 SANTA CRUZ) ×1000 under oil, demonstrating VEGF expression in the podocytes. FIG. 10C shows ELISA of protein extracted from renal cortex from transgenic and wild type mice using VEGFₓₓₓb specific ELISA (R&D Systems), p<0.01 (ANOVA).

FIG. 11 illustrates podocyte-specific VEGF₁₆₅b over-expression reduced glomerular water permeability. The normalized glomerular ultra-filtration coefficient was significantly reduced in mice with heterozygous and homozygous VEGF-A₁₆₅b over-expression. The reduced glomerular water permeability in VEGF165b over-expressing heterozygous mice was rescued by incubation with VEGF₁₆₅. Paired measurements were done on the same glomerulus before and after treatment. Bars represent mean±S.E.M. LₚA/Vᵢ measurements. Numbers in parentheses represent number of glomeruli studied. *=p<0.05 compared with wild type littermate and ††=p<0.001 compared with wild type. ##p <0.001 compared with heterozygous. One-way ANOVA with Bonferroni correction. ‡=p<0.05 compared with untreated gloms from the same NephVEGF165b transgenic mice (paired t test).

FIG. 12 illustrates glomerular VEGF-A₁₆₅b over-expression does not significantly affect renal function. Renal function and proteinuria were determined in transgenic mice and wild type littermates by measuring (A) plasma [creatinine] (μmol/L), (B) plasma [Urea] mmol/L (C) protein:creatinine ratio of urine collected using metabolic cages (mg/mmol).

FIG. 13 illustrates exogenous application of VEGF₁₆₅b to individual control WT glomeruli decreased the normalised glomerular ultrafiltration coefficient. FIG. 13A shows populations of normal WT glomeruli were exposed for 60 mins to control solution, 40 pM recombinant human VEGF₁₆₅b to 1 nM VEGF₁₆₅b. Bars represent mean±S.E.M. LₚA/Vi measurements. Numbers in parentheses represent number of glomeruli studied. *=p<0.05 compared with control. Kruskal-Wallis one-way ANOVA with Dunnett's correction. FIG. 13B shows contrasting properties of rhVEGF₁₆₅b on normalised LpA/Vi of isolated single murine glomeruli (FIG. 13Bi) and previously published data (reference 39) of similar concentrations of rhVEGF₁₆₅ on rat glomeruli (FIG. 13Bii) using the same assay.

FIG. 14 shows over-expression of human VEGF₁₆₅b (heterozygous & homozygous) in the podocyte causes a reduction in endogenous VEGF expression. Protein was extracted from Wild-type, heterozygous and homozygous kidneys using RIPA buffer supplemented with sigma protease inhibitor cocktail. Endogenous mouse VEGF was measured in the tissue homogenate using ELISA (R&D MOUSE VEGF). The ELISA picks up mouse VEGF₁₂₀ and VEGF₁₆₄ and has low cross reactivity with human VEGF (0.2%). *p<0.05 Kruskil Walis with Dunns post hoc test.

FIG. 15 illustrates ultra-structural phenotype. FIG. 15A shows haematoxylin and eosin light microscopy normal in all animals. FIG. 15B shows electron microscopy revealed that no significant difference in the area of the GFB enclosed by sub-podocyte space (SPS) was seen (WT-littermate [WT] controls 55±5% vs Homozygous 41±10%, p=NS). However a marked reduction in fenestration number and size was observed in homozygous animals FIG. 15C). EM studies above used 70 nm sections. FIG. 15D shows “Closed” Fenestrations containing electron dense material were prominent in homozygous animals (arrow) contrasting with more conventional, rare diaphragmed fenestrations (see FIG. 15E-arrow).

FIG. 16 shows ultra-structural phenotype on vascular side of glomerular basement membrane of homozygous animals, fenestration parameters determined using 40 nm sections. FIG. 16A shows an example of prominence (9/11) of “closed” fenestrations in the glomeruli from homozygous transgenic animals. FIG. 16B shows fenestration density was significantly reduced in both functional areas of the GFB. FIG. 16C shows percentage of “Closed” fenestrations as a proportion of total fenestrations. FIG. 16D shows open fenestration width. FIG. 16E shows “closed” fenestration width. WT=wild-type littermate controls, Hom=homozygous for transgene. SPS covered GFB=areas of GFB through which filtrate has to exit a restrictive sub-podocyte space to reach Bowman's space. Non-SPS covered GFB=areas of GFB through which filtrate enters Bowman's space directly.

FIG. 17 illustrates constitutive podocyte-specific VEGF₁₆₅b-overxpressing heterozygous transgenic mice are resistant to the glomerular lesion associated with STZ-induced diabetes. Groups of 12 week old heterozygous VEGF₁₆₅b mice age matched with WT littermate controls (n=5 each group) received 100 μg/gram body weight/day for 3 days (200 μL injection volume). Controls received equal volume of citrate buffer. Fasting (1 hour fast) blood glucose, urinary protein/creatinine ratio and body weight were monitored every 2 weeks. At 6 weeks post induction animals were put into metabolic cages and 12 hour urine collections made and urinary albumin content assayed by ELISA. *p<0.05 compared with WT diabetic, ANOVA, Bonferroni. Blood glucose levels for STZ groups were similar STZ WT: 22.97±1.47 mmol/L vs STZ HET: 26.42±1.45 mmol/L (p=NS).

FIG. 18 illustrates that VEGF-A₁₆₅b inhibits neovascularization in the oxygen induced retinopathy model, but does not block revascularization.

FIG. 19 illustrates that VEGF-A₁₆₅b inhibits human retinal endothelial cell migration.

FIG. 20 illustrates that VEGF-A₁₆₅b is a survival factor for human endothelial cells.

FIG. 21 illustrates that VEGF-A₁₆₅b is a cytoprotective agent for RPE cells.

FIG. 22 illustrates that VEGF-A₁₆₅b is an endogenous survival factor.

### DETAILED DESCRIPTION

### EXAMPLE 1

Materials and Methods

Tissue Source

Human adult renal cortex was collected from the normal pole of unilateral, unipolar renal carcinoma nephrectomy specimens with local ethics committee approval (Bristol). Three human female fetuses of 10 and 12 weeks pregnancy were obtained with local ethics committee approval (Leiden).

Immunohistochemistry and ELISA

Sections were microwave heated in 0.01 mM citric buffer saturated sodium citrate pH buffer (pH 6.0) for either 12 minutes at 95° C. (VEGFₓₓₓb), or for 7 minutes at 800 W followed by 9 minutes at 120 watts (pan-VEGF staining). Sections were washed twice with PBS, incubated with 3% hydrogen peroxide solution for 20 minutes, washed again, blocked with 10% BSA (Sigma;A4378) in 0.05% Tween-PBS (TBS) and then with 1.5% normal horse serum (NHS Vector lab; S-2000) in TBS (1 hr). Sections were incubated with 8 μg/mL primary antibody (MAB3045, R&D Systems, Sigma; I8765, or Santa Cruz, 7269) in TBS (pH7.4) with 1% BSA, washed twice with TBS, blocked again then incubated with secondary (Vector Lab; BA2000, 1:200 dilution in NHS in TBS for one hour washed twice, then incubated with Vectastain ABC solution (Vector Lab; PK4000) for 45 minutes.

Cytotoxicity, ELISA Flow Cytometry and Migration Assays

VEGF ELISA[21], cytotoxicity[12], apoptosis[22], and migration[23] were determined as described in the referenced literature.

Culture of Glomerular Endothelial Cells (GEnC)

GEnC derived from decapsulated glomeruli isolated from normal human kidney (according to the supplier's data sheet) were obtained at passage 2 from the Applied Cell Biology Research Institute (ACBRI, Kirkland, USA). Cells were cultured in EGM2-MV (endothelial growth medium 2-microvascular, Cambrex, Wokingham, UK), made up from EBM2 (endothelial basal medium 2, Cambrex) and fetal calf serum (FCS, 5%), antimicrobial agents and growth factors as supplied. Cells being prepared for, or being used in, experiments were cultured in EGM2-MV without VEGF.

Measurement of Trans Endothelial Electrical Resistance (TEER)

TEER is a measure of ion flux and is inversely related to the fractional area of pathways open to water and small molecules across a cell monolayer. Tissue culture inserts containing polycarbonate supports (0.4 μm pore size, Nalge Nunc International, Rochester) were seeded with GEnC at 100,000 cells/cm². Measurement of TEER of GEnC monolayers was performed using an Endohm 12 electrode chamber and EVOMx voltmeter (World Precision Instruments, Sarasota, USA) as previously described [24]. Medium was replaced with serum-free medium (EBM2). Baseline TEER was measured after 1 hr and the culture medium was again replaced, this time with SFM alone (control) or containing 1 nM VEGF₁₆₅ (R&D Systems) or 1 nM VEGF₁₆₅b. TEER was remeasured at 15, 30 and 60 minutes. Previous work has demonstrated a peak response to VEGF between 30 and 60 minutes in this assay.

Results

VEGFₓₓₓb Expression in Adult Renal Cortex

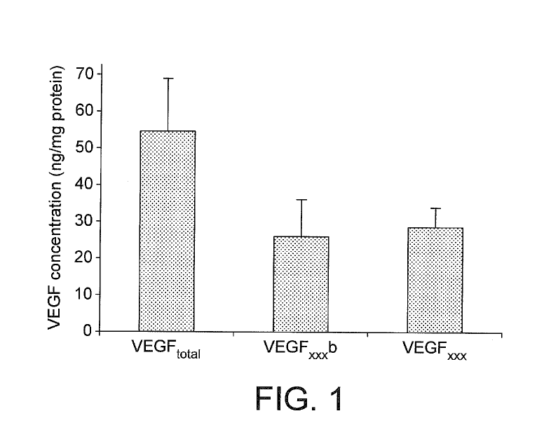

To determine quantitatively the contribution of VEGFₓₓₓb isoforms to the total VEGF expression in normal adult kidneys, VEGFₓₓₓb and total VEGF was measured in protein extracted from freshly frozen renal cortex. Total protein was measured using the commercially available ELISA, and VEGFₓₓₓb levels measured by a comparable ELISA but using a biotinylated detection antibody specific to the C terminus of VEGFₓₓₓb. Total VEGF concentrations in normal renal cortex averaged of 54.2±14 ng/mg protein. VEGFₓₓₓb concentrations averaged 25.8±9.6ng/mg (n=3, FIG. 1), or 45±5% of the total VEGF. This was similar to the relative proportion of total VEGF that was VEGF₁₆₅b measured in protein extracted from normal isolated glomeruli collected from human nephrectomy specimens (46.6±18%, n=3).

VEGFₓₓₓb Staining in Adult Kidney

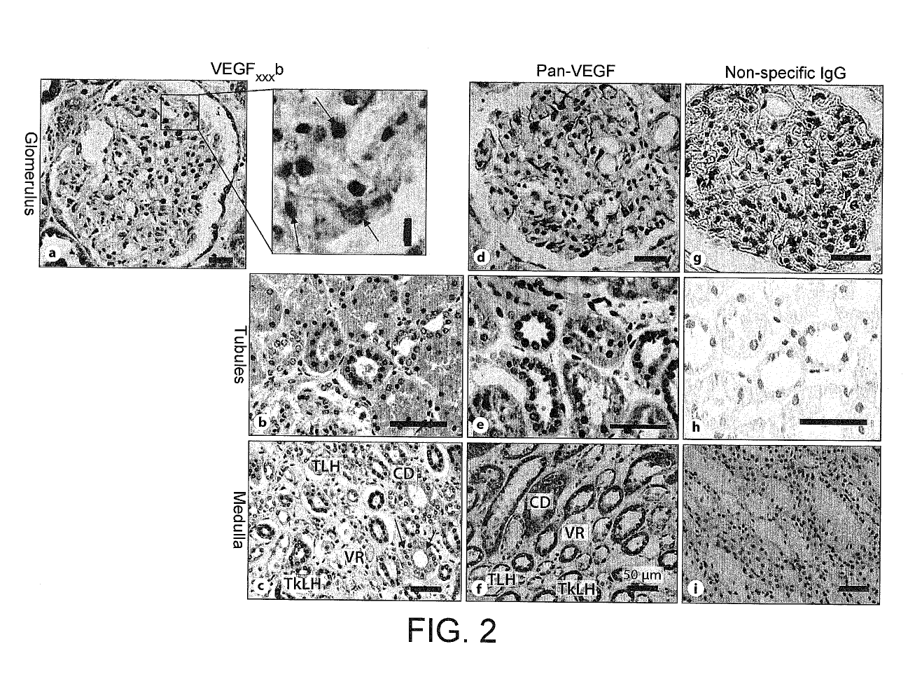

The antibody to VEGFₓₓₓb used for immunohistochemistry is an affinity purified mouse monoclonal IgG₁ antibody, Cat MAB3045, commercially available through R & D Systems, which has been characterised previously [15,16,25]. It binds recombinant VEGF₁₆₅b, and shows expression of VEGF₁₆₅b, VEGF₁₈₉b, VEGF₁₂₁b VEGF₁₈₃b and VEGF₁₄₅b collectively termed VEGFₓₓₓb, but not VEGF₁₆₅. Western blotting has previously shown that all the proteins recognised by this antibody are also recognised by commercial antibodies raised against VEGF-A. This antibody does not recognise the VEGFₓₓₓ isoforms, but does recognise recombinant VEGF₁₆₅b and VEGF₁₂₁b, conclusively demonstrating that this antibody is specific for VEGFₓₓₓb [16]. VEGFₓₓₓb staining was limited to a significant proportion of podocytes (FIG. 2A, see arrows), but present in parietal epithelial cells, macula densa and proximal and distal tubules of the renal cortex (FIG. 2B). VEGFₓₓₓb staining was also observed in the vasa recta, collecting ducts and ascending thin and thick loop of Henle (FIG. 2B). In the epithelial cells of the ascending thick loop of Henle, strong intracellular staining was observed, whereas in the epithelial cells of the collecting ducts, staining was highly localised to the tips of the apical surface (blue arrow) and to the basolateral cytoplasm (black arrow, FIG. 2C). A similar trend was observed for pan-VEGF staining (FIG. 2D-F). Staining was never seen when an isotype matched IgG antibody was used as a control (FIG. 2G-I) under the same conditions.

VEGFₓₓₓb Staining in Developing Glomerulus

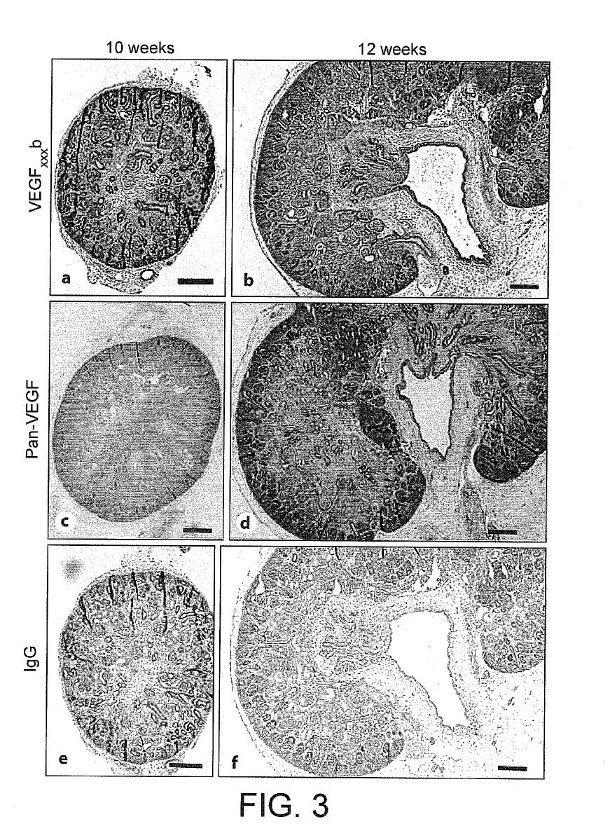

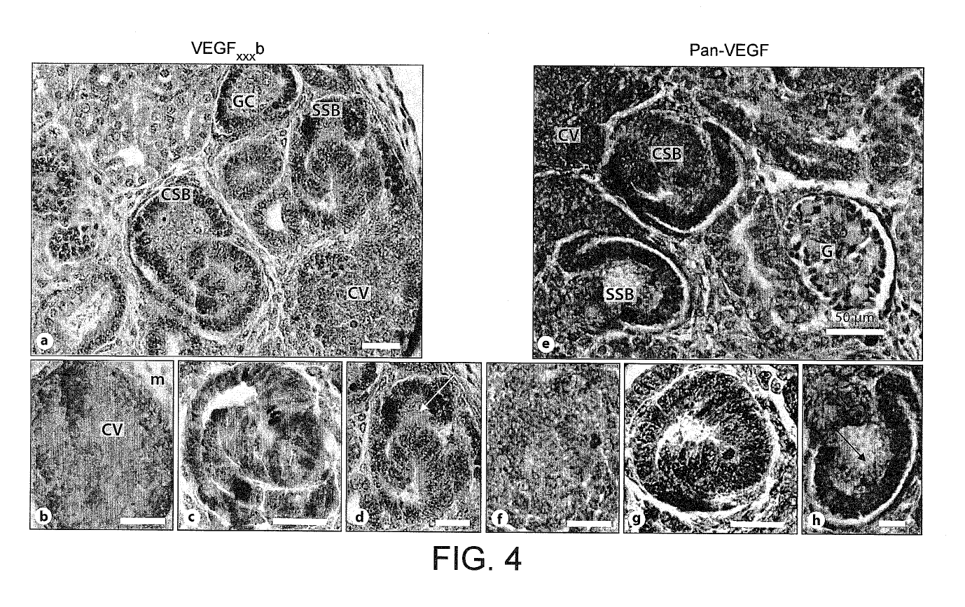

To investigate VEGF₁₆₅b expression in the developing glomerulus, immunohistochemistry was carried out on sections of human fetal renal tissue. Immunohistochemical staining for VEGFₓₓₓb of 10 and 12 week old fetuses showed clear expression in the developing nephron that was noticeably stronger than the surrounding mesenchyme (FIG. 3A, 10 weeks and FIG. 3B 12 weeks). Staining was very intense in all stages of nephrogenesis from the condensed vesicle stage onwards (FIG. 4A-D). Staining with an antibody to all isoforms of VEGF confirmed that VEGF was located throughout the developing kidney (FIG. 3C, 10 weeks & FIG. 3D, 12 weeks). Interestingly, there were no areas of the kidney that stained for VEGFₓₓₓb but did not stain for pan-VEGF. In contrast, there were a number of areas, including in the mesenchyme, where VEGFₓₓₓb antibodies did not detect expression, but the pan-VEGF antibody did (FIG. 4A versus FIG. 4E). No staining was seen using any isotype-matched affinity purified mouse IgG (FIG. 3E, 10 weeks and FIG. 3F, 12 weeks).

In the condensed vesicle (FIG. 4B, cv) VEGFₓₓₓb staining was greater than the surrounding mesenchyme (FIGS. 4A and 4B, m). The greatest intensity of staining could be seen during epithelialisation and staining was highest in both the apical and basolateral parts of the primitive epithelial cell and weakest in the nuclear regions, indicating a cytoplasmic subcellular localisation. Pan-VEGF staining was also apparent in these regions in the condensed vesicle (FIGS. 4E and 4F). As development proceeded to comma (FIG. 4C, G) and S-shaped bodies (FIG. 4D, H), VEGFₓₓₓb staining (FIGS. 4C and D) became even more restricted to the apical and basolateral parts of the primitive columnar epithelial cells of the developing nephron, as did pan-VEGF staining (FIGS. 4G and H). Of note was the more diffuse VEGFₓₓₓb staining in the glomerular cleft (FIG. 4D, H, arrows), the site at which endothelial cells will invade.

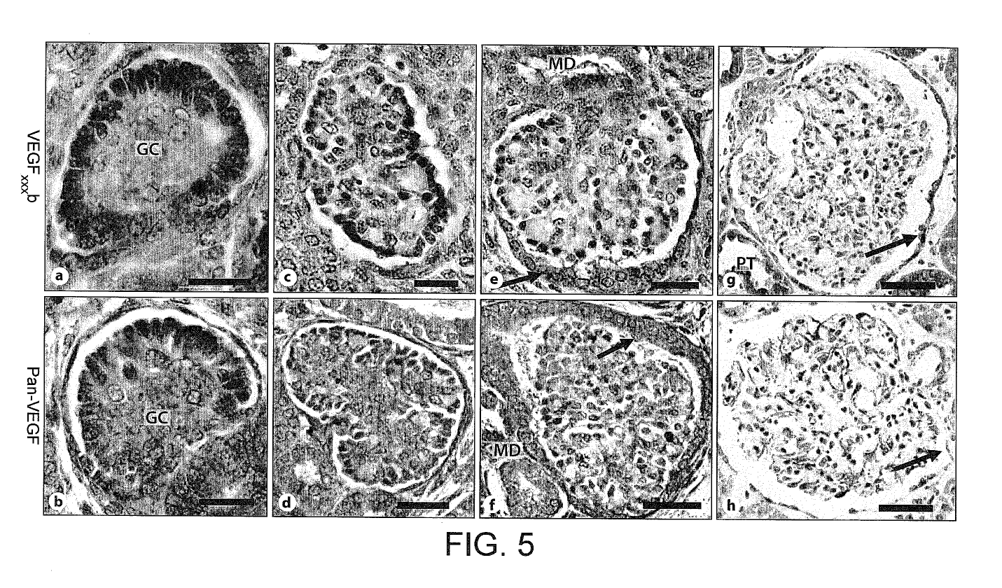

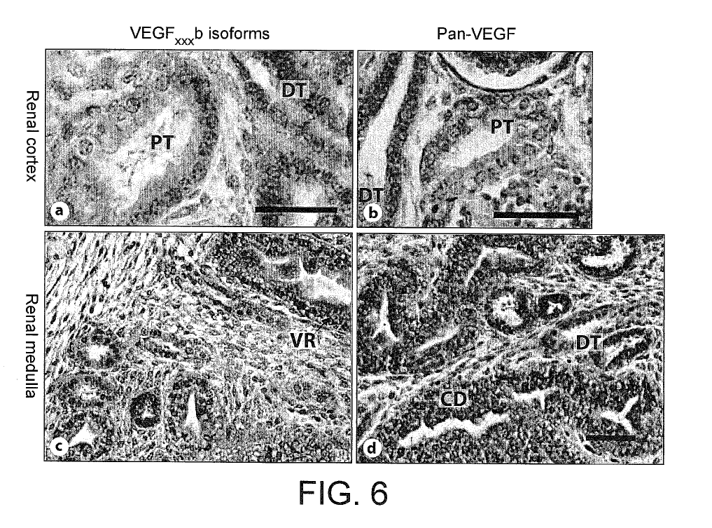

This pattern of staining, observed in the primitive epithelial cells and glomerular cleft appeared to be more diffuse in the capillary loop stage of glomerulogenesis (FIG. 5A), in contrast to pan-VEGF which appeared stronger (FIG. 5A in comparison with 5B contrasts with 4E in comparison with 4F). As the glomerulus was formed (FIG. 5C), VEGFₓₓₓb staining appeared to diminish in the developing glomerular visceral epithelial (podocytes) and endothelial cells (FIG. 5E), but there was still marked staining in the parietal epithelial cells lining Bowman's capsule, and cells of the maculae densa (FIG. 5E). Pan-VEGF expression appeared to be maintained through glomerular maturation, and stained up more glomerular epithelial cells than VEGFₓₓₓb (FIGS. 5D, F). In comparison, in the mature glomeruli, VEGFₓₓₓb staining was limited to a subpopulation of podocytes (FIG. 2A). This appeared to be true for pan-VEGF staining too, but the pan-VEGF antibody identified more podocytes than the VEGFₓₓₓb antibody (FIG. 2D)

VEGFₓₓₓb Staining in Developing Tubules of the Primitive Renal Cortex

Throughout the developmental stages examined VEGFₓₓₓb staining was clearly seen in both the proximal and distal portions of the convoluted tubules (FIG. 6A). More specifically, staining was seen both in the apical and basolateral parts of the primitive epithelial cells. In addition, staining was seen in all areas of tubule development in the renal cortex. Comparable pan-VEGF staining was seen in the tubules of the developing renal cortex (FIG. 6B).

VEGFₓₓₓb Staining in the Primitive Renal Medulla

VEGFₓₓₓb staining appeared to more specifically localised in the primitive epithelial cells of the developing nephron (FIG. 6C), whereas pan-VEGF staining was intense and widespread throughout the renal medulla (FIG. 6D), VEGFₓₓₓb staining was seen in both the apical and basolateral sides, but not as intense in the central, perinuclear regions in the epithelial cells of the distal tubules extending into the medulla and of the collecting ducts (FIG. 6C). Furthermore, it appeared that where the more distal portions of the convoluted tubules differentiate into their specialised transporting segments, (the thin and thick loop of Henle), VEGFₓₓₓb staining was less intense (FIG. 6C). Weak VEGFₓₓₓb staining was also observed the endothelial cells of the vasa recta (FIG. 6C).

The Effect of VEGF₁₆₅b on Human Glomerular and Endothelial Cells in vitro

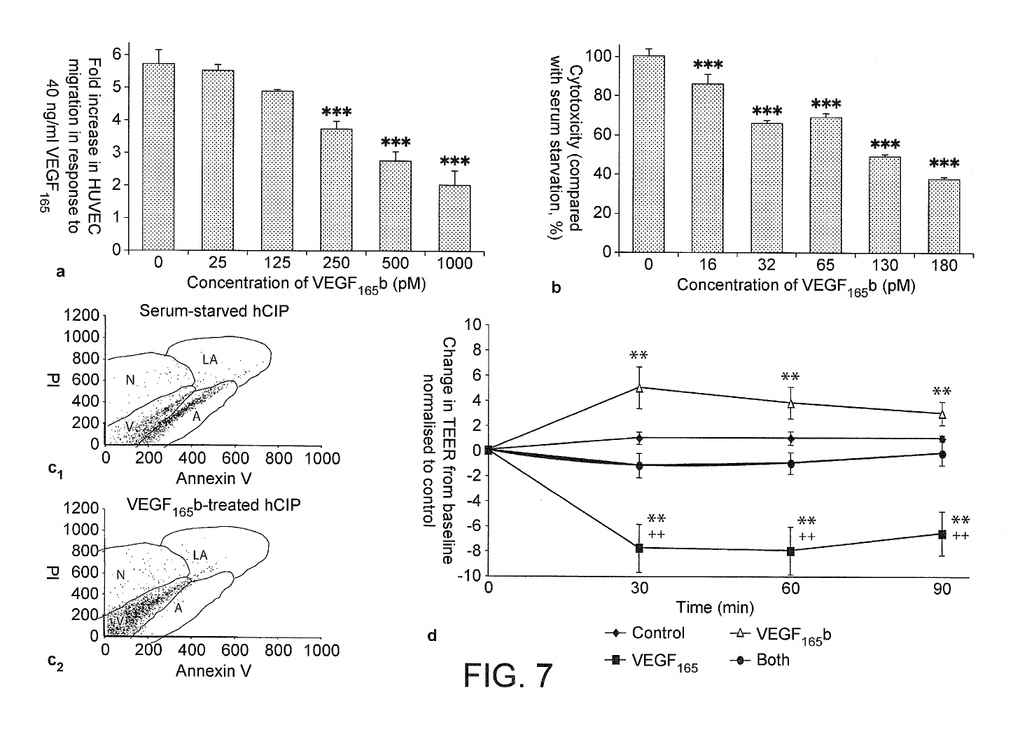

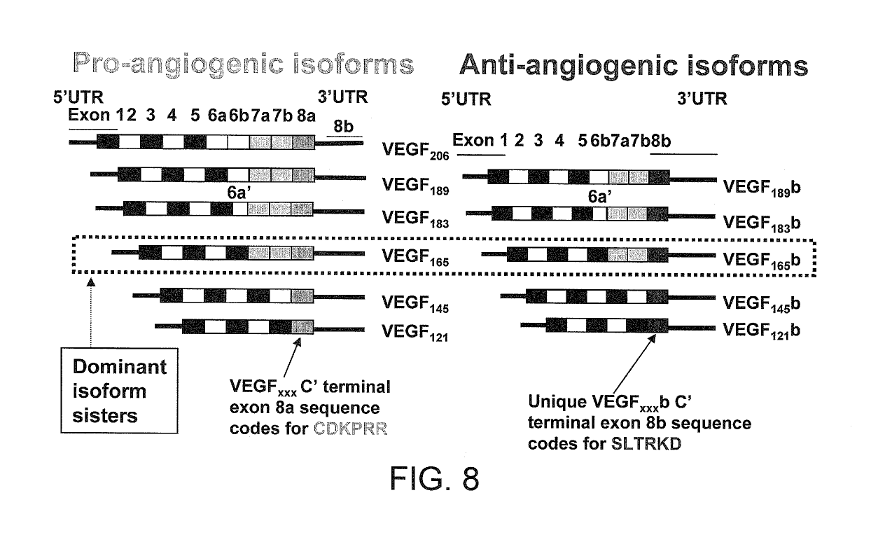

Alterations in expression may reflect changes in function in the embryo, in the adult and in disease. The role of VEGFₓₓₓb in the developing human kidney is not known. Although VEGF₁₆₅b has been shown to inhibit endothelial cell migration in response to VEGF₁₆₅, it is not known whether this inhibition can be balanced by controlling the expression level of VEGF isoforms. To determine whether VEGF₁₆₅b could dose dependently affect endothelial cells in vitro the effect of VEGF₁₆₅b on human endothelial cell migration was estimated. FIG. 7a shows that VEGF₁₆₅b dose dependently inhibited HUVEC migration responses to VEGF₁₆₅, with an IC₅₀ of 0.29±0.03 fold excess (i.e. 40 ng/ml VEGF₁₆₅ was 50% inhibited by 11.4±1.4ng/m1 VEGF₁₆₅b, n=3). This is consistent with downregulation of VEGF₁₆₅b during the endothelial invasion phase of glomerular development. To determine whether VEGF₁₆₅b might have positive benefits during glomerular development, we measured the effect of VEGF₁₆₅b on podocyte cytotoxicity. VEGF₁₆₅b dose dependently decreased cytotoxicity of primary cultured podocytes (FIG. 7b) with an EC₅₀ of 107±1.2 pM, showing that VEGF₁₆₅b had a cytoprotective effect (n=8). The LDH assay measures only the number of cells releasing a cytoplasmic protein and hence does not distinguish between apoptosis and necrosis. Interestingly, VEGF₁₆₅b did not affect podocyte cell proliferation (16.5±1.1×10³ cpm/cell compared with 15.8±1.0×10³ cpm/cell, n=6), thus suggesting an anti-apoptotic effect on human podocytes. This was confirmed by flow cytometry using AnnexinV and propidium iodide staining (FIG. 7c). Whereas serum starvation induced a significant proportion of the cells to undergo apoptosis (region A in FIG. 7Ci), this was inhibited by treatment with 0.3 nM VEGF₁₆₅b (FIG. 7cii). To determine whether VEGF₁₆₅b could affect glomerular endothelial barrier function a trans-electrical endothelial resistance (TEER) in vitro assay of glomerular endothelial permeability was used. Although VEGF₁₆₅ significantly reduced glomerular endothelial TEER (indicating an increase in monolayer permeability), VEGF₁₆₅b resulted in a significant increase in TEER, and VEGF₁₆₅b inhibited the increase induced by VEGF₁₆₅ (FIG. 7d, n=4). Thus in contrast to VEGF₁₆₅, VEGF₁₆₅b prevents endothelial cell migration and reduces monolayer permeability, while maintaining podocyte cell survival in vitro.

Discussion

The role of VEGF in renal function and development has been the subject of intense scrutiny since VEGF expression was demonstrated in the renal cortex and medulla by antibody staining, RT-PCR, in situ hybridisation and Northern blotting, in both normal and disease states. VEGF is highly expressed in the kidney—more so than nearly any other tissue, but very few studies have accounted for the VEGFₓₓₓb variants that are anti-angiogenic [16,20,25]. mRNA encoding the VEGFₓₓₓb splice variants were first described in normal renal cortex, and VEGF₁₆₅b protein was first identified in human podocytes by isoform specific siRNA [26]. The experiments described here, however, are the first to quantitate the contribution of VEGFₓₓₓb to the total VEGF expressed. The finding that, in normal renal cortex, almost half of the VEGF found is VEGFₓₓₓb, has significant implications for our interpretation of the many studies that have investigated VEGF expression in normal renal tissues and disease states [15]. The finding that VEGFₓₓₓb isoforms are a highly significant component of the total VEGF in renal tissues implies an as yet unknown physiological relevance.

Pan-VEGF and VEGFₓₓₓb Staining Patterns Compared

VEGF-A, both mRNA and/or protein, of unknown isoform family has been detected in the presumptive and mature podocytes and primitive columnar epithelial cells of the developing nephron, in both rodent and human tissues [2,4-7,27,28]. In this study we sought to determine the presence and localisation of VEGFₓₓₓb proteins in human metanephric kidneys and compare its spatiotemporal staining pattern to that detected by pan-VEGF antibodies. We detected VEGFₓₓₓb in metanephric kidneys from 10 and 12 week fetuses using immunohistochemical staining. Our pan-VEGF staining of the metanephric kidney is in close agreement with previous studies; VEGF was detected in presumptive and mature podocytes and to the primitive columnar epithelial cells of the nephron [2,4-7,29]. VEGFₓₓₓb isoforms appear to be present in a subset of cells that express VEGF, as there were no areas in the metanephric kidney that stained for VEGFₓₓₓb isoforms but not for pan-VEGF but some areas that stained positively for pan-VEGF but not for VEGFₓₓₓb. In the adult kidney, the presence of VEGF in the convoluted tubules is in contrast to in situ hybridisation studies, which show in adult tissues the primary source of renal VEGF synthesis to be the podocyte [30] suggesting the possibility of glomerular derived VEGF protein uptake by tubular cells.

Glomerulogenesis and VEGFₓₓₓb

Glomerulogenesis is dependent on reciprocally inductive interactions between renal endothelial cells and nephron epithelial cells, but although various genes [31-34] and growth factors [9,11,35-37] have been implicated at specific stages, the molecular regulators of the cell differentiation events are poorly understood. A dosage sensitivity to VEGF exists within the developing glomerulus [8], similar to that seen when VEGF expression was manipulated throughout the embryo [38,39]. As VEGF₁₆₅b has been shown to counteract some of the effects of VEGF₁₆₅, and has a dose dependent effect on podocyte survival, it is likely that dosage sensitivity of glomerulogenesis to VEGFₓₓₓb may also be a critical component of normal renal cortex formation, and a recent study showing that transgenic mice over-expressing VEGF₁₆₅b in mouse podocytes have reduced glomerular permeability characteristics supports this suggestion[40].

Previous Studies on VEGF

Apart from the original isolation of VEGF₁₆₅b mRNA from renal cortex [14] and protein in the glomeruli [16], and the identification of VEGF₁₆₅b mRNA and protein in differentiated, but not proliferating conditionally immortalised podocyte cell lines [26], previously used methodologies either did not detect VEGFₓₓₓb isoforms (RT-PCR using primers in the proximal part of exon 8), or did not distinguish VEGFₓₓₓb isoforms from VEGFₓₓₓ isoforms. The only study that has addressed this examined microdissected mRNA from fetal, child and adult glomeruli, and found that expression of VEGF₁₆₅b mRNA was lower in the S and C shaped bodies than in adult or child glomeruli. The decreasing protein expression we see here from condensed vesicle through S and comma shaped bodies to immature glomeruli may therefore be a result of this endogenous downregulation at the mRNA level, temporally shifted slightly, as the VEGF protein is turned over more slowly than the mRNA. Schumacher et al also noted higher VEGF₁₆₅b expression in the adult glomeruli compared with VEGF₁₆₅. Unfortunately antibodies that specifically detect VEGFₓₓₓ isoforms are not yet available, but it appears likely that most of the VEGF staining in adult glomeruli is VEGF₁₆₅b. Interestingly, in that study, Schumacher et al demonstrate a complete loss of VEGF₁₆₅b in Denys-Drash glomeruli, indicating a link to WT1 [20], a finding recently confirmed by over-expression studies in vitro [41]. Podocyte specific knockout of VEGF during development resulted in a lack of formation of glomeruli and renal failure immediately after birth followed by death within 6 hours [8], presumably because endothelial cells fail to migrate into the glomerulus (as is evidenced by a lack of phenotypically discernable endothelial cells in the glomerulus), and thus aberrant microvessel formation and glomerular filtration. VEGF knockouts, however, also are VEGFₓₓₓb knockouts, so it is not clear which part of the phenotype is dependent on VEGFₓₓₓb knockout. Inhibitors of VEGF, such as VEGF-TRAP [42], sFlt-1[43], bevacizumab [44] and other monoclonal antibodies to VEGF, shown to affect renal function, are also likely to affect the VEGFₓₓₓb isoforms. Therefore it is not clear whether the results in studies previously carried out on the inhibitory role of VEGF in glomerular function were due to the pro-angiogenic isoforms, or the anti-angiogenic isoforms, or both.

Possible Functions of Renal VEGFₓₓₓb Expression

VEGF₁₆₅b inhibits VEGF₁₆₅-mediated endothelial cell proliferation and migration in vitro and vasodilatation in isolated arteries ex vivo [14,16], VEGF₁₆₅-mediated physiological angiogenesis in the mesentery and the eye [16], the chicken chorioallantoic membrane and the dorsal skin chamber in mice [18], pathological VEGF-mediated angiogenesis in tumour models [16], and hypoxia-driven retinal angiogenesis in the eye in vivo [17]. VEGF₁₆₅b has been shown to have both dominant negative and partial agonist activity on endothelial mediated signalling [18] [16], potentially explaining its ability to both inhibit migration and protect against cytotoxicity. In contrast no effect of VEGF₁₆₅b was seen on glomerular endothelial monolayer integrity in vitro (FIG. 7C). In the developing kidney VEGFₓₓₓ isoforms are thought to mediate endothelial cell survival and migration, microvascular permeability [9] and perhaps epithelial cell survival [12]. The results shown here are consistent with the concept that the VEGFₓₓₓb isoforms also support epithelial cell survival, without increased permeability, and are downregulated during endothelial cell migration presumably to allow invasion into the glomerular cleft.

Eremina et al [8] have shown that unrestricted expression of VEGF₁₆₅ during development is significantly detrimental, which taken together with these results suggest a balance of pro-angiogenic/anti-angiogenic VEGF-A is required for normal development and function [8,9,31,33,45,46]. Expression of VEGFₓₓₓb isoforms and crucially the control of distal and proximal 3′end splicing control during kidney development may therefore play a significant role in the modulation of VEGFₓₓₓ driven responses. VEGFₓₓₓb may play a modulatory role in the developing kidney. For example, factors must limit and halt the endothelial cell invasion into the glomerular cleft at the primitive glomerulus and subsequent stages of glomerular development. To address this hypothesis further investigation is required including conditional transgenic knock-out and over expressing models that are designed to take account of both sides of the VEGF-A biology—angiogenesis and permeability—and perhaps, more importantly, their inhibition.

Conclusion

In this Example, we examined expression of VEGFₓₓₓb in metanephric kidneys from human fetuses, and performed parallel in vitro experiments to understand the role of VEGFₓₓₓb on cell types involved in glomerular function. VEGFₓₓₓb formed 45% of total VEGF protein in adult renal cortex, and VEGF₁₆₅b does not increase glomerular endothelial cell permeability, inhibits migration, and is cytoprotective for podocytes. During renal development, VEGFₓₓₓb was expressed in the condensed vesicles of the metanephros, epithelial cells of the comma shaped bodies, invading endothelial cells and epithelial cells of the S shaped body, and in the immature podocytes. Expression reduced as the glomerulus matured. These results show that the anti-angiogenic VEGFₓₓₓb isoforms are highly expressed in adult and developing renal cortex, and suggest that the VEGFₓₓₓb family plays a role in glomerular maturation and podocyte protection by regulating the pro-angiogenic pro-permeability properties of VEGFₓₓₓ soforms.

### EXAMPLE 2

Materials and Methods

Animal Maintenance

All transgenic (TG) lines were generated on the C57BL6xCBA/CA background. Animal care and procedures were carried out within United Kingdom Home-Office protocols and guidelines. Transgenic mice were crossed with mice in C57BL6 background. For permeability experiments, F₂₋₃ generation male transgenic mice were selected and wild type littermates were used as controls for the heterozygous mice.

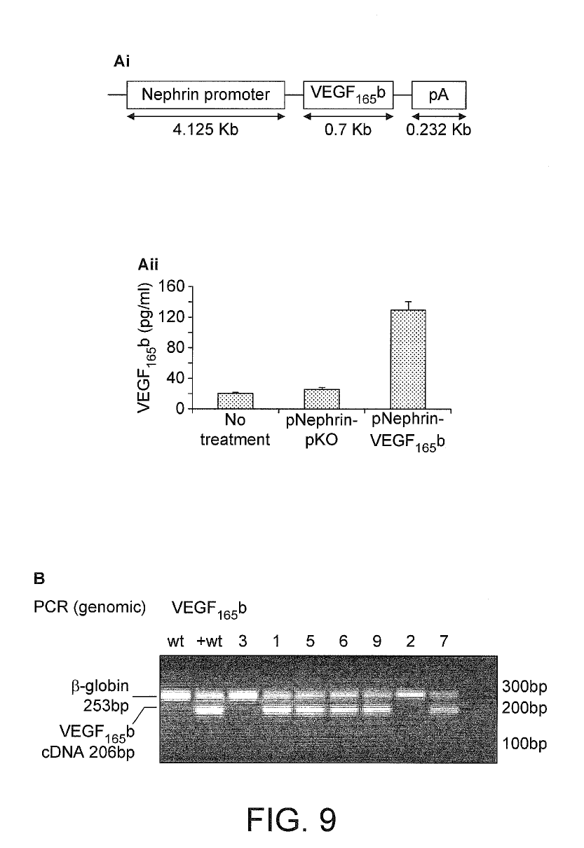

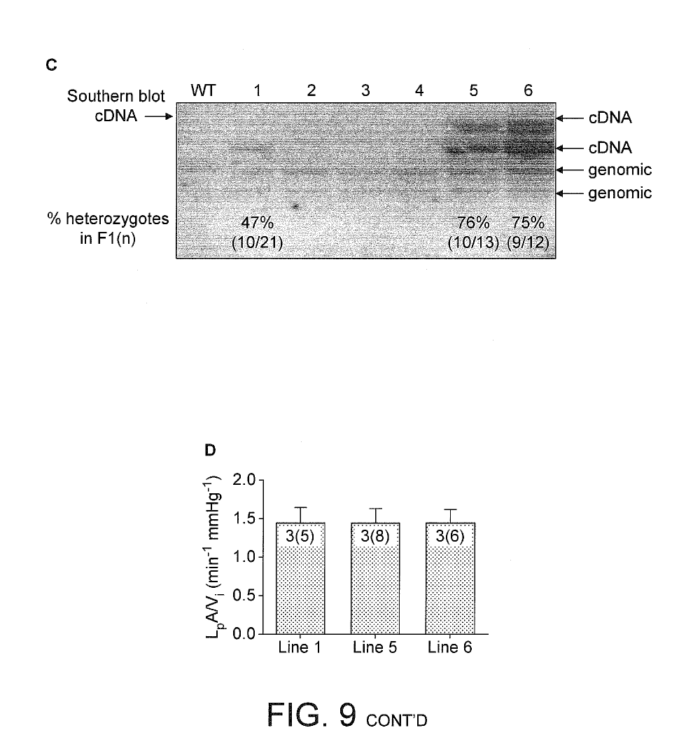

Construction of pNephrin-VEGF₁₆₅b (FIG. 9Ai)

pcDNA3-VEGF₁₆₅b was cloned as previously described (1). To generate a plasmid with mouse nephrin promoter upstream of VEGF₁₆₅b cDNA and poly-A signal, pcDNA3-VEGF₁₆₅b was digested with HindIII to delete CMV promoter. Mouse Nephrin promoter (kindly supplied by Professor Susan Quaggin) was from plasmid 5′-Nephrin-pKO. 5′-Nephrin-pKO was digested with Pac I and Xho I enzymes followed with both ends blunted. To get Hind III linkage ends, the blunted DNA product was ligated with phosphorylated HindIII linkers, followed by digestion with the HindIII enzyme. The nephrin promoter DNA fragment was inserted with rapid DNA ligation kit (Roche Applied Science) into the linearised pcDNA3-VEGF₁₆₅b of which the CMV promoter has been deleted and the colonies with correct orientation were selected (FIG. 9Ai).

Podocyte Transfection

Human conditionally immortalised visceral glomerular epithelial cells (hCIPs) previously characterised (39) were kindly donated by Professor Moin Saleem. hCIPs were cultured in RPMI 1640 medium with insulin, transferrin, selenite (all Sigma, Dorset, UK), and 10% fetal calf serum at 33° C., 5% CO₂. For transfection experiments, HCIPs were cultured to about 50% confluence, equal amount of pNephrin-VEGF₁₆₅b and empty vector 5′-Nephrin-pKO were transfected into HCIPs using the Lipofectamine Reagent (Invitrogen) according to the manufacturer's instructions. Expression of VEGF₁₆₅b in the supernatant of control, mock transfected and pNephrin-VEGF₁₆₅b transfected podocytes were analysed by VEGFₓₓₓb-family specific ELISA (R&D Systems DY304E)(FIG. 9Aii).

Generation of Transgenic Mice

The DNA fragment of mNephrin-VEGF₁₆₅b-pA for microinjection was generated with HindIII and HaeII digestions of pNephrin-VEGF₁₆₅b, gel-purified using QIAEX II DNA Extraction kit (QIAGEN, UK) before final purification with elutip minicolumns (Schleicher & Schuell biosciences) according to the manufacturer's suggestion. Microinjection of purified DNA into embryos was carried out by B&K Universal Ltd., UK. Briefly, 5-10 ng/μl of purified DNA fragment was microinjected into the pronuclei of fertilised one-cell stage embryos obtained from young C57BL6xCBA/CA mice. Successful injected embryos were cultured overnight in M16 medium (Sigma-Aldrich, UK) at 37° C., 5% CO₂ and transplanted into oviducts of pseudo-pregnant mice in C57BL6xCBA/CA background the next day. After pups weaned, genomic DNA (gDNA) extracted from tail biopsies were screened for the existence of transgene via polymerase chain reaction (PCR) (FIG. 9B) and confirmed by Southern blotting (FIG. 9C). Animals were bred to homozygosity using a standard breeding/genotyping programme. Too generate homozygous animals siblings (founder line 1) were crossed and subsequent pups of the F1 generation, themselves crossed with wild-types, were also genotyped. For homozygous animals all subsequent pups from at least 3 litters and a minimum of 20 pups were required to carry the transgene and all pups from subsequent litters. Numbers of animals for functional phenotype analysis was determined by the numbers required to demonstrate statistical analysis (from previous data showing that to demonstrate a significant difference in glomerular LpA/Vi of 25% the use of a minimum of 3 animals and 5 glomeruli was required), restrictions of UK home office license and ethical review board.

PCR

PCR was performed as shown in our previous publication (Qiu Y et al, 2008, Faseb J. 2008, 22(4), 1104-12). Briefly, one pair of primers (forward primer sequence: 5′-TCA GCG CAG CTA CTG CCA TC-3′ (SEQ. ID. NO: 1) and reverse primer sequence: 5′-GTG CTG GCC TTG GTG AGG TT-3′ (SEQ. ID. NO:2)) gave rise to a PCR product of 208 by to detect specifically the transgene. Another pair of primers (forward primer: 5′-ACG TCC TAA GCC AGT GAG TG-3′ (SEQ. ID. NO: 3) and reverse primer: 5′-CAG CCT TCT CAG CAT CAG TC-3′ (SEQ. ID. NO:4)) for mouse 13-globin resulting in a band of 253 by was also included in this amplification, serving as internal control. Each reaction contained 2 μl of the 10× buffer, 0.2 mM dATP, dGTP, dCTP and dGTP, 1.5 mM MgCl₂, 500 nM forward and reverse primers, 0.5 units of Taq polymerase (Abgene, UK), 0.5 μl gDNA and water to 20 μl. PCR was initiated with 94° C. for 4 mins, followed by 35 cycles of denaturation at 94° C. for 30 secs, annealing at 62° C. for 30 secs and extension at 72° C. for 30 secs, a final extension at 72° C. for 10 mins to finish.

Southern Blotting

10-15 μg of tail gDNA was digested with EcoRI restriction enzyme. DNA was separated on 0.8% Agarose gel, denatured and capillary-transferred to Hybond N⁺ membrane (Amersham, UK). DNA was fixed with baking at 80° C. for 2 hours. Membranes were probed with an alkaline phosphatase-labelled DNA fragment, exactly the same as the one used for microinjection. Probe preparation and transgene detection followed the manufacturer's guideline of Gene Images Alkphos Direct Labelling and Detection System (Amersham, UK).

RT-PCR

RT-PCR was carried out as shown in our previous publication (Qiu Y et al, 2008. Faseb J. 2008, 22(4), 1104-12). Briefly, total RNA was isolated with Trizol (Invitrogen) extraction and DNase I (Invitrogen) digested as manufacturer's suggestion to prevent gDNA contamination. 1 μg of DNase-treated RNA was reverse transcribed into cDNA with AMV reverse transcriptase using standard method as suggested by the manufacturer (Promega). Both cDNA and RNA treated with DNase I were subject to PCR with forward primer 5′-ACA AGA TCC GCA GAC GTG TA-3′ (SEQ. ID. NO: 5) and reverse primer 5′-ACA GAT GGC TGG CAA CTA GA-3′ (SEQ. ID. NO: 6). PCR amplification was initiated with 94° C. for 4 mins, 35 cycles of 94° C. for 30 secs, 50° C. for 30 secs and 72° C. for 30 secs, followed by final extension at 72° C. for 10 mins. A band at 199 by indicated VEGF₁₆₅b transgene expression.

Enzyme-Linked Immunosorbant Assay (ELISA) of VEGFₓₓₓb

Tissue protein lysate was prepared from mouse kidney tissue in RIPA buffer. For cultured podocytes, conditioned medium from cells with or without transfection was used. Protein concentration was determined by Bio-rad assay (Bio-rad) and the amount of VEGF₁₆₅b was determined by ELISA as previously described with a specific detection antibody against VEGFₓₓₓb isoforms.

Briefly, 0.08 μg of goat anti-VEGF polyclonal IgG (AF293-NA, R&D Systems) diluted in 1× PBS (pH 7.4) was adsorbed onto each well of a 96-well plate (Immulon 2HB, Thermo Life Sciences, Basingstoke, UK) overnight at room temperature. The plate was washed three times between each step with 1× PBS-Tween (0.05%). After blocking with 100 μl of 5% BSA in PBS for 1 h at 37° C., 100 μl of recombinant human VEGF₁₆₅b (R&D Systems) diluted in 1% BSA in PBS (ranging from 62.5 pg/ml to 4 ng/ml) or protein samples were added to each well. After incubation for 1 h at 37° C. with shaking and three washes, 100 μl of mouse monoclonal anti-VEGFₓₓₓb biotinylated IgG (clone 264610/1, R&D Systems) at 0.4 μg/ml was added to each well, and the plate left for 1 h at 37° C. with shaking. 100 μl of streptavidin-HRP (R&D Systems) at 1:200 dilution in 1% BSA in PBS was added, the plate left at room temperature for 20 mins and 100 μl/well O-phenylenediamine dihydrochloride solution (Substrate reagent pack DY-999; R&D Systems) added, protected from light and incubated for 20 mins at room temperature. The reaction was stopped with 50 μl/well 1 M H₂SO₄, and absorbance read immediately in the Opsys MR 96 well plate reader (Dynex Technologies, Chantilly Va., USA) at 492 nm, with control reading at 460 nm.

Glomerular Permeability

The normalised glomerular ultrafiltration coefficient (LₚA/Vᵢ) of isolated intact whole glomeruli was calculated using an oncometric technique first described by Salmon et al 2006 (40).

Glomerular Isolation and Solutions

Mice aged between 8 and 10 months were killed by cervical dislocation and kidneys removed. Glomeruli were isolated in mammalian ringer solution containing 1% bovine serum albumin (BSA) using conventional techniques. The glomerular harvest retained by the 100 μm mesh sieve was kept on ice to preserve morphology. During isolation the concentration of plasma proteins within the glomerular capillaries equilibrates with the surrounding solution. Perifusate containing either dilute BSA (1%) or concentrated BSA (8%) was made in mammalian ringer solution and adjusted to pH 7.45±0.02.

Apparatus

Micropipettes, pulled from glass capillary tubes (o.d. 1.2 mm; Clark Electromedical Instruments, Reading, UK). The 13 μm aperture tip was fitted within a rectangular cross section glass microslide (i.d. 400 μm×4 mm; Camlab, Cambridge, UK). The microslide was visualised over the 10x objective using an inverted microscope. (Leica DM IL HC Fluo) A monochrome video camera (Hitachi KP-M3AP) was attached to the top of the microscope to permit recording of individual glomeruli loaded into the system. The video camera was connected through a digital timer (FOR.A VT33) to a video cassette recorder (Panasonic AG7350) and monochrome monitor. (Sony SSM-125CE) Perifusates were held in elevated heated reservoirs connected to the microslide via tubing. A rapid-response remote tap (075P3;Bio-Chem Valve, Inc) controlled the choice of perifusate exciting the microslide. The fluid within the system was maintained at 37° C. using a separate system of tubes and heating coils connected to a heated water bath.

Glomerular Volume Change

Glomeruli that were free of Bowmans capsule and arteriolar or tubular fragments were chosen for study. All glomerular observations were performed within 3 h of nephrectomy. After a period of equilibration in flowing dilute perifusate (2 minutes) the rapid remote tap was switched allowing the concentrated BSA to excite the microslide.

Analysis of Glomerular Volumetric Change

Perifusate switches were recorded on videotape and sequences reviewed off line using Apple imovies (Apple USA) and an analogue to digital converter (ADVC-300, Canopus). All measurements were done by operator blind to the genotype or treatment. A sequence of images straddling the time point at which perifusate switch occurred, was created. The glomerular image in each was replicated in Adobe Photoshop CS3 (Adobe Systems, Inc., CA, USA) and the area (A μm²) calculated using image J (US National Institutes of Health). Glomerular volume was derived from area measurements by substituting glomerular image area (A) into the formula

Glomerular volume=4/3 πr³

(where r=glomerular radius) to reveal:

Glomerular volume=[4/3A((A/π))1/10⁻⁶

Glomerular volume (nl) was plotted against time since the first appearance of the Schlieren phenomenon marking the arrival of the new oncopressive perifusate. Two regression lines were then applied to these points. The slope of the first was set as zero and applied to points before the solution switch when glomerular volume was stable. The two lines were calculated to meet at their breakpoint. This point was defined as the time point at which glomerular volume begins to decline. The second line was applied to points covering a time period of at least 0.04 s and no more than 0.1 s from the breakpoint. Within these confines the points at which the applied regression line had the greatest slope were chosen.

Calculation of LₚA

The slope of the second regression line describes the greatest initial rate of glomerular volume change and can therefore be equated to the term Jᵥ in the Starling equation:

Jᵥ/A=Lₚ[P_{c}−Pᵢ)−σ(Π_{c}−Πᵢ)]

The net hydrostatic pressures acting across an isolated glomerulus can be assumed to be negligible. Previous work suggests the reflection coefficient of an isolated glomerulus is not significantly different from 1. The Starling equation can therefore be rearranged to show that

LₚA=Jᵥ/−ΔΠ

(in nlmin⁻¹ mmHg⁻¹) (where ΔΠ is the difference between capillary and interstitial oncotic pressure).

VEGF₁₆₅b Experiments

In a separate group of experiments glomeruli from wild type C57/Blk6 mice were exposed to recombinant human VEGF₁₆₅b (rhVEGH₁₆₅b; PhiloGene, Inc., NJ, USA). After isolation glomeruli were incubated at 37° C. in 1% BSA solution or 1% BSA solution containing either 40 pm VEGF₁₆₅b or 1 nM VEGF₁₆₅b. Glomeruli from each solution were then individually loaded into the microslide and the ultrafiltration coefficient calculated as described above.

Phenotype and Histological Analysis

A separate group of animals aged between 8 and 10 months was used to collect tissue, plasma and urine for phenotypic and histological analysis. Animals were individually housed in metabolic cages for up to 12 h to obtain a urine sample. They were anaesthetised using 5% isoflurane and a blood sample taken by direct cardiac puncture. Mice were then culled by cervical dislocation. The kidneys were removed, divided and preserved by immersing in either 4% PFA, 2.5% gluteraldehyde or liquid nitrogen.

Immunohistochemistry

Kidney samples from wildtype, heterozygous and homozygous mice were formalin-fixed and embedded in paraffin. 5 μm sections were mounted onto gelatin/poly-1-lysine-coated glass slides. The sections were dried onto the slides in a 37° C. incubator overnight. Sections were dewaxed in Histoclear (RA Lamb, Eastbourne, UK) for 5 min and rehydrated through graded ethanol solutions (100, 90, and 70% v/v). Sagittal sections of all kidneys were cut and stained (H&E). These were coded and reviewed by 2 assessors independently. Assessors were unaware of the origin of the section and could not distinguish between animals on glomerular size, mesangial matrix, glomerular cellular scores or tubular morphology.

Microwave antigen retrieval was performed in 0.01 mM citric buffer, saturated sodium citrate pH buffer (pH 6.0), for 7 min at 95° C. at 800 W followed by 9 min at 120 W. Sections were cooled to room temperature prior to being washed twice in deionized water for 5 min each time. Sections were incubated with freshly prepared 3% v/v hydrogen peroxide (BDH, Poole, UK) diluted in 1× PBS for 5 min, then washed twice for 5 min with 1× PBS and blocked with 5% w/v BSA (Sigma)) followed by 1.5% w/v normal goat serum (Vector Laboratories) in 5% w/v BSA for 30 min. The sections were washed twice with 0.05% v/v PBS-Tween at room temperature for 5 min, then incubated with the primary antibody diluted in 1.5% w/v normal goat serum in 1× PBS. A polyclonal rabbit VEGF antibody (A20 sc152; Santa Cruz Biotechnology, Inc., Santa Cruz, Calif., USA) was used. Tissue sections were treated with a matched concentration of normal, affinity-purified rabbit IgG (Sigma), used as a negative control. The sections were washed twice in 0.05% v/v PBS-Tween, for 5 min each time. The blocking step was repeated as before, followed by two 5min washes in 0.05% v/v PBS-Tween. All sections, including the controls, were incubated with biotinylated goat anti-rabbit IgG (Vector Laboratories) diluted in 1.5% w/v normal goat serum for 1 h in a humid chamber at room temperature. Sections were washed twice with 0.05% v/v PBS-Tween, 5 min per wash, then incubated with a pre-prepared avidin-biotinylated enzyme complete kit (Vector Laboratories) for 45 min in a humid chamber at room temperature. Again, the sections were washed twice with 0.05% v/v PBS-Tween, 5 min each time, followed by incubation with 3,3′-diaminobenzidine substrate (Vector Laboratories) to yield a brown-colored product. The reaction was stopped by washing twice with deionized water for 5 min. Sections were counterstained with Mayer's hematoxylin (BDH) for 5 min, then differentiated in water. Sections were dehydrated by passing through increasing concentrations of ethanol (70, 90, and 100% v/v) for at least 2 min each, cleared in xylene for at least 10 min, and permanently mounted in DPX mountant for histology. Staining was examined with a Nikon Eclipse E-400 microscope; images were captured using a DCN-100 digital imaging system (Nikon Instruments).

Electron Microscopy Analysis

Kidney fixation procedures were adapted and modified from Hayat (23). Portions of kidney from each mouse were rapidly excised and sliced in a pool of glutaraldehyde fixative (2.5% glutaraldehyde in 0.1 M cacodylate buffer [pH 7.3], 4-8° C.). Cubes (0.5 to 1 mm diam.) of kidney cortex were further fixed at 4° C. with glutaraldehyde fixative. After a minimum of 3 hour fixation, the tissues were left in fresh fixative overnight, then washed in cacodylate buffer postfixed for 1 hour in osmium (1% osmium tetroxide in 0.1 M cacodylate buffer, pH 7.3, 4° C.). Tissues were washed in cacodylate buffer and then distilled water before ethanol dehydration, infiltration and embedding in Araldite resin (Agar Scientific, Stansted, UK). Glomeruli were identified from 0.5 μm Toluidine Blue stained survey sections. Glomeruli were cut at 70-100 nm thick for EM observation. Analysis was conducted on digital electron micrographs (taken at 890 and 2900 times). Measurements were made of %coverage of the glomerular filtration barrier by the sub-podocyte space (SPS), thickness of the Glomerular Basement Membrane, height of SPS, foot process width (or separation between slit diaphragms) and separation between endothelial fenestrations and width of fenestrations. Linear measurements from electron micrographs were made at random points using a Photoship grid. In order to clarify changes in fenestrations 40 nm sections were used.

Murine Specific VEGF ELISA

Kidney tissue protein lysate was prepared from transgenic and control mice and total protein quantified as described above. Mouse VEGF-A concentration was measured in duplicate for each sample using a commercial enzyme-linked immunosorbent assay kit (Quantikine® R&D Systems; Minneapolis, Minn.) that recognizes the soluble isoforms VEGF₁₂₀ and VEGF₁₆₄. Microplates were precoated with a monoclonal antibody specific for VEGF. Recombinanat mouse VEGF was diluted to concentrations ranging from 250 pg/mL-7.8 pg/mL . Standards and samples were pipetted into the wells. Any VEGF-A present in the sample, was bound by the immobilized antibody. After any unbound substances were washed away, an enzyme-linked polyclonal antibody conjugated to horse radish peroxidase and specific for VEGF was added to the wells. After a wash to remove any unbound antibody-enzyme reagent, 100 μl/well O-phenylenediamine dihydrochloride solution (Substrate reagent pack DY-999; R&D Systems) was added to each well, the plate was protected from light using foil and incubated for 20 min at room temperature. The reaction was stopped with 50 μl/well 1 M H₂SO₄, and absorbance was read immediately in the Opsys MR 96-wellplate reader (Dynex Technologies, Chantilly, Va., USA) at 492 nm, with control reading at 460 nm).

Glomerular Filtration Rate.

GFR was determined in anaesthetizsed 9 month old heterozygous and age-matched littermate controls using a single bolus injection of FITC-Inulin.

Conditionally Immortalized Human Glomerular Endothelial Cells (ciGEnC).

ciGEnCs are well characterized and were grown and maintained as previously described(48). For PV-1 western blot experiments were grown at 33° C. for 6 days then 37° C. for 5 days. Cells were serum starved for four hours then treated with either 1 nM VEGF165 or 1 nM VEGF165b or a combination of 1 nM VEGF165 & 1 nM VEGF165b or left untreated for 24 hours. Renal cortex, glomerular lysate from 125 m and 180 m sieves and podocyte lysate were loaded as controls. Experimental protocol was as previously described (1) with primary anti-body concentration 1:200 (anti-PV-1), secondary antibody concentration 1:10,000).

Statistics

Figures are given as mean+/−standard error. p<0.05 was regarded as significant. Methods of statistical analysis are included in relevant figure legend as stated above in the section “Brief Description of the Drawings”.

Results

Generation of pNeph-VEGF₁₆₅b Heterozygous Transgenic Mice

VEGF₁₆₅b cDNA was cloned into an expression vector under the control of the Nephrin promoter (FIG. 9Ai). To assess transfection and construct functionality, human conditionally immortalized podocytes, were then transfected with the expression construct, and VEGF₁₆₅b expression was assessed in the cell supernatant at 48 hours. Significantly more VEGF₁₆₅b was seen in the transfected podocytes compared with control vector, or untransfected cells (FIG. 9Aii). Potential founder lines were identified by PCR (FIG. 9B) and Southern blot analysis screening of pups born from injected embryos (FIG. 9C). These founder lines were then used for subsequent studies. There was no difference in isolated glomerular functional phenotype (LpA/Vi: permeability to water-area product) between founder lines (FIG. 9D).

VEGF₁₆₅b Expression in Renal Cortex of pNeph-VEGF₁₆₅b Heterozygous and Homozygous Transgenic Mice

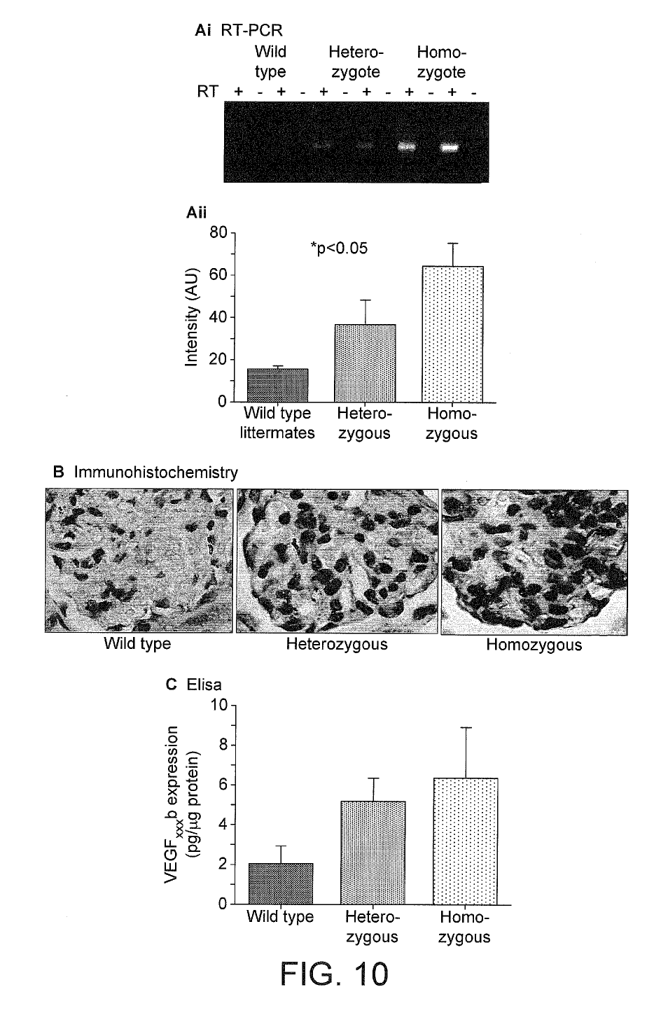

VEGF₁₆₅b expression was determined in the renal cortex of transgenic mice and age matched littermate wild-type controls using 3 methods. Firstly, Exon 8b specific RT-PCR of renal cortex for transgene (FIGS. 10Ai and Aii) p<0.05, chi squared test for trend. N=3 per group. Secondly, by immunohistochemistry (FIG. 10B) using an anti-human anti-VEGF antibody, which demonstrated increasing IHC staining for VEGF in the podocytes, and finally, by exon 8b-specific ELISA (FIG. 10C) on protein extracted from renal cortex from transgenic and wild type mice using VEGFₓₓₓb specific ELISA (p<0.01, ANOVA). All 3 methods demonstrated a gradient of expression from wild-type littermate controls through heterozygous transgenic mice to homozygous animals.

Functional Phenotype: Podocyte-Specific VEGF₁₆₅b Over-Expression Reduces Glomerular Water Permeability and Urinary Protein Loss (Summary Table 1)

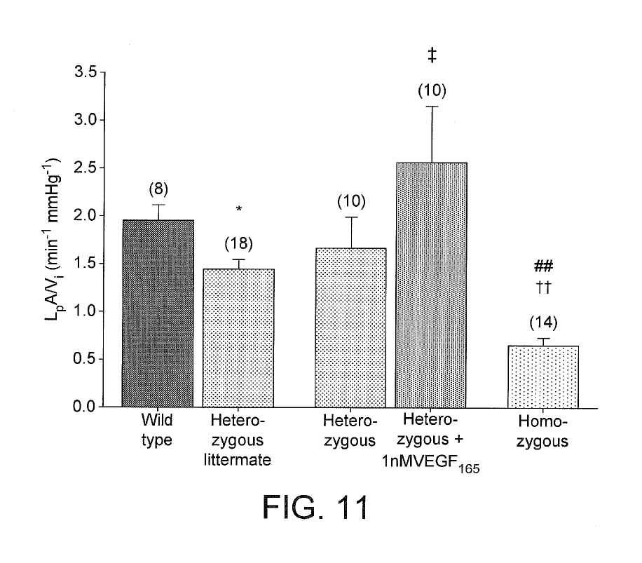

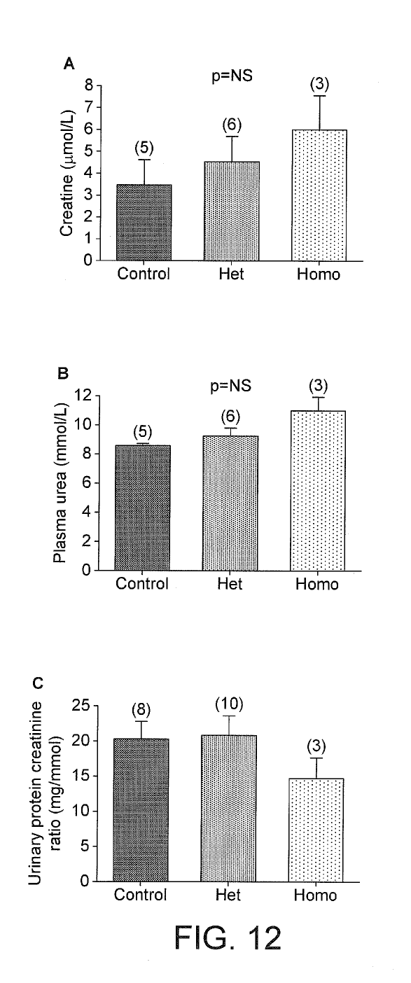

To determine whether the permeability to water of the glomeruli from transgenic animals was altered by VEGF₁₆₅b over-expression, the normalized glomerular ultra-filtration co-efficient (LₚA/Vᵢ) was investigated (using a validated oncometric assay we have previously characterized (40)) in groups of glomeruli from WT, heterozygous and homozygous VEGF₁₆₅b over-expressing mice. A marked difference was seen in LpA/Vi between these three groups (FIG. 11) from 1.95±0.16 nl.min⁻¹mmHg ⁻¹ in WT to 1.43±0.1 in heterozygous to 0.67±0.07 in homozygous mice, table 1). To determine whether this reduction was attenuated by exogenous VEGF₁₆₅, LpA/Vi from glomeruli from transgenic mice was measured, and then the glomeruli exposed to 1 nM VEGF₁₆₅ for 1 hr. FIG. 11 shows that this restored LpA/Vi to levels similar to wild type. LₚA is a permeability-area product so glomerular capillary volume was therefore calculated in glomeruli from controls and transgenics. No significant difference was seen: WT (mean 0.98±0.16), transgenic mean 0.81±0.11, p>0.4), suggesting that changes in LpA/Vi are due to changes in water permeability alone rather than reduced glomerular capillary area due to developmental abnormalities. We also estimated VEGF₁₆₄ expression in transgenic mice and wild-types and found no evidence of compensatory increase in VEGFₓₓₓ isoforms (data not shown). Plasma creatinine, urea levels and GFR (306.7±57.52 1.11/min, n=4, WT-controls vs 344.1±41.80 μl/min, n=4 Heterozygotes) were not significantly different. Plasma creatinine and urea levels were not significantly different in transgenic mice and wild type littermates (FIGS. 12A and 12B). However, urinary protein:creatinine ratio (uPCR) of urine collected using metabolic cages showed lower values in the homozygous animals (FIG. 12C and Table 1) compared to WT controls but did not rech significance. Body weight of animals and blood glucose levels were also unchanged in transgeneic mice.

TABLE 1

| | Wild-type | Heterozygous | Homozygous |
|---|---|---|---|
| | littermate control | animals | animals |
| Glomerular | 0.98 ± 0.16 | 0.73 ± 0.07 | 1.56 ± 0.11 |
| Volume (ul) | n = 8 | n = 18 | n = 36 |
| LₚA | 1.93 ± 0.32 | 1.00 ± 0.12 | 1.06 ± 0.16 |
| | n = 8 | n = 18 | n = 23 |
| LₚA/Vᵢ | 1.95 ± 0.17 | 1.44 ± 0.11 | 0.67 ± 0.066 |
| | n = 8 | n = 18 | n = 23 |
| uPCR (ng/nnol) | 20.16 ± 2.55, | 20.74 ± 2.80 | 14.80 ± 2.78 |
| | n = 8 | n = 10 | n = 3 |
| Plasma Creatinine | 3.4 ± 1.21, | 4.5 ± 1.2 | 11.07 ± 0.90 |
| (μl/mol/L) | n = 5 | n = 6 | n = 3 |
| Plasma Urea | 8.6 ± 0.10, | 9.32 ± 0.50, | 11.07 ± 0.90, |
| (mmol/L) | n = 5 | n = 6 | n = 3 |
| GFR (μl/min) | 306.7 ± 57.5, | 344.1 ± 41.8, | |
| | n = 4 | n = 4 | |
| % SPS coverage | 55 ± 5 | ND | 41 ± 10 |
| Foot process width | 400 ± 40 | ND | 460 ± 70 |
| (nm) | | | |
| GBM thickness | 248 ± 10 | ND | 252 ± 9 |
| under SPS (nm) | | | |
| GBM thickness | 196 ± 6 | ND | 240 ± 14, |
| uncovered | | | *p < 0.05 |

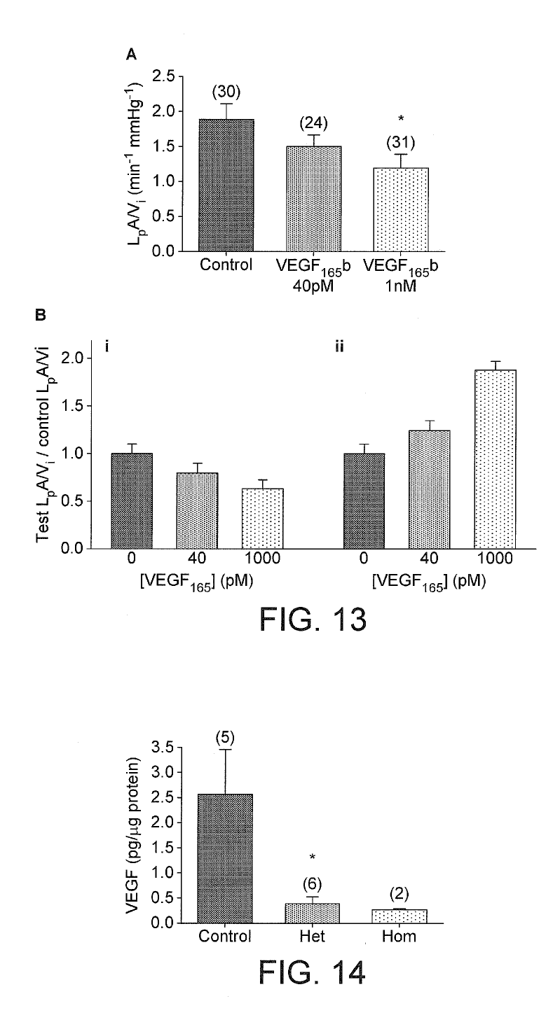

To assess whether exogenous administration of rhVEGF₁₆₅b could reproduce the reduction in LₚA/Vᵢ WT glomeruli were incubated with increasing doses of rhVEGF₁₆₅b. Exogenous VEGF₁₆₅b significantly reduced ultrafiltration co-efficient in a dose dependent fashion (FIG. 13A). FIGS. 13Bi and Bii summarizes the characteristically distinct permeability changes induced in LₚA/Vᵢ elicited by VEGF₁₆₅ (increase)(FIG. 13Bii) and VEGF₁₆₅b (decrease) (FIG. 13Bi).

Ultra-Structural Phenotype: Podocyte-Specific VEGF₁₆₅b Over-Expression Reduces Fenestral Size and Density

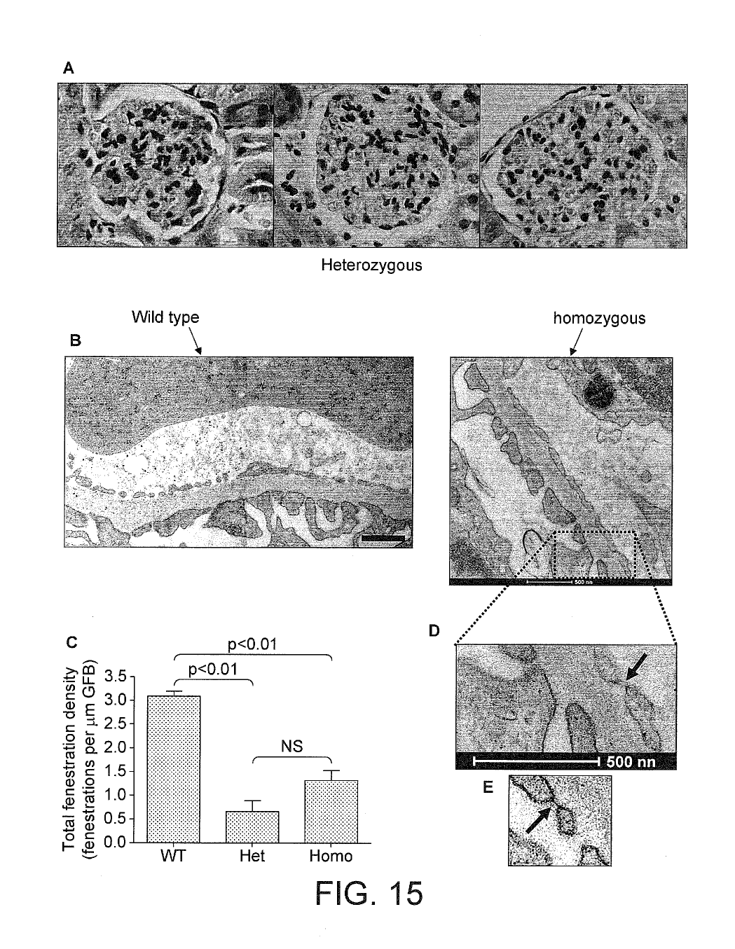

Macroscopically the mice were normal up to 18 months of age with normal behaviour, growth rate, feeding and no urinary sediment. Histological assessment with light microscopy revealed no obvious abnormality between the wild types (WTs) and transgenic animals (FIG. 15A). However, serial transmission electron microscopy (TEM) studies revealed that typical glomerular endothelial open fenestrations were difficult to identify at all in homozygous animals (FIG. 15B).

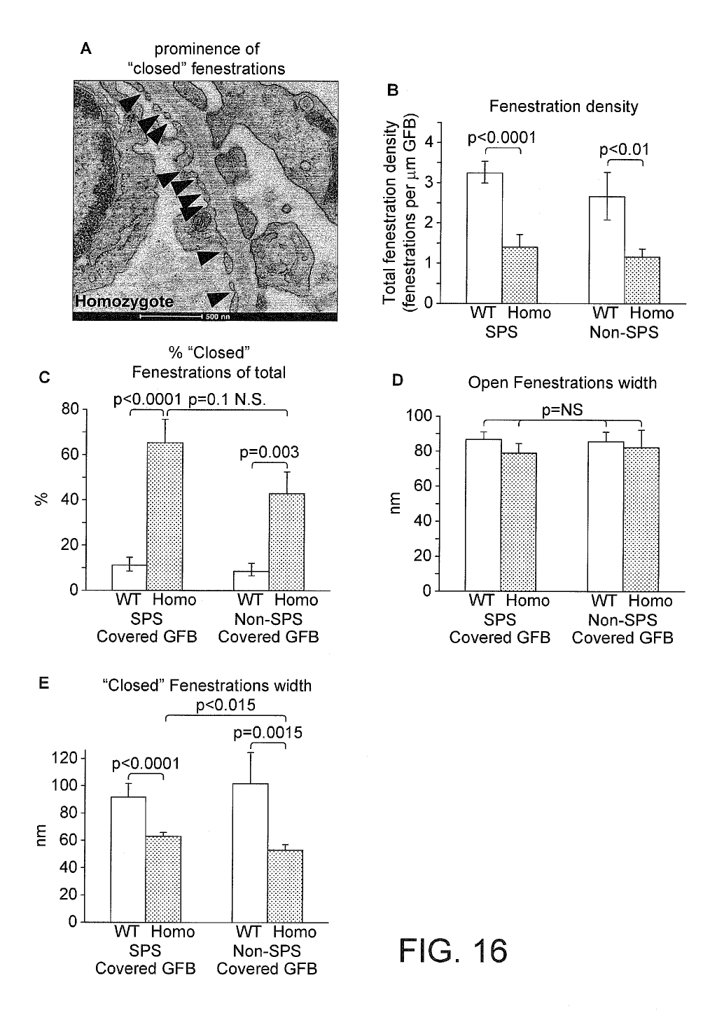

Ultra-structural measurements revealed no change in sub-podocyte space coverage, foot-process width and GBM thickness in SPS covered areas (Table 1). However, the GBM in areas of the GFB devoid of SPS coverage were significantly thinner in WT controls(196±6 nm) vs homozygous animals(240±14 nm), p<0.01. In addition fenestration density was reduced in homozygous animals (FIG. 16C, Table 1). Moreover, the vastas expected, the vast majority of fenestrations in WT-littermate controls did not demonstrate fenestral diaphragms. In contrast, many of the fenestrations in the homozygous transgenic animals contained electron dense material (FIG. 16D) that contrasted with the conventional (but rare) distinct diaphragms seen in normal glomerular endothelium in vivo (FIG. 16E). Since, it was not possible to define whether these fenestrations contained atypical diaphragms, excess glycocalyx, glycocalyx-like material, “sieve plugs” or was simply a reflection of smaller fenestrations with more frequent (relatively) sectioning through the attenuated edge of fenestrations, we termed these “closed” fenestrations. Further EM characterisation was therefore performed using 40 nm sections from paired samples fixed and processed in parallel from homozygous animals and littermate controls. Random measurements (in excess of 200 from multiple animals) were made using a photo shop grid (FIG. 16).

On the urinary side of the GBM the podocyte foot process slit-diaphragm width and density was not significantly different (Table 1). In contrast on the vascular side there was a significant increase in the proportion of “closed” fenestrations (FIGS. 16A and 16B). Furthermore, although the open fenestrations were of a similar size in WT-controls and homozygous animals (FIG. 16C), the closed fenestrations were significantly narrower (FIG. 16D).

Although a detailed study of the nature of the “closed” fenestrations was not possible we did attempt to clarify if the over-expression of VEGF₁₆₅b had influenced PV-1 (Plasmalemma vesicle protein-1) expression. We were unsuccessful using the established antibodies for immunogold studies and we therefore studied PV-1 expression by western blotting in conditionally immortalised glomerular endothelial cells. These studies did not show any significant change in PV-1 expression at the protein level in glomerular endothelial cells exposed to VEGF₁₆₅b.

Resistance to Diabetic Glomerular Lesion

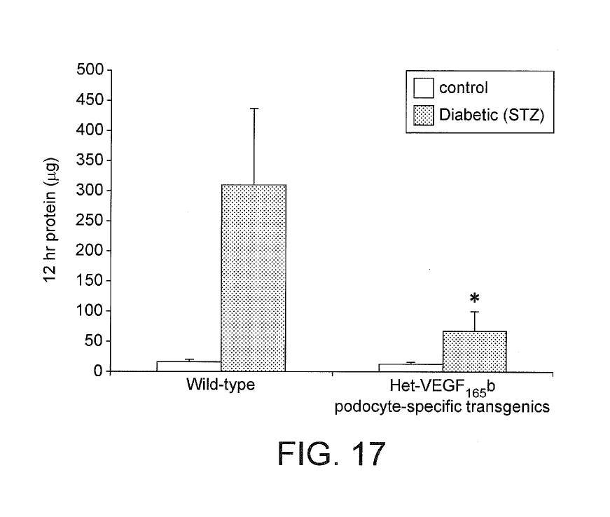

FIG. 17 illustrates constitutive podocyte-specific VEGF₁₆₅b-overxpressing heterozygous transgenic mice are resistant to the glomerular lesion associated with streptozotocin (STZ)-induced diabetes.

Groups of 12 week old heterozygous VEGF₁₆₅b mice age matched with WT littermate controls (n=5 each group) received 100m/gram body weight/day for 3 days (200 μL injection volume). Controls received equal volume of citrate buffer. Fasting (1 hour fast) blood glucose, urinary protein/creatinine ratio and body weight were monitored every 2 weeks.

At 6 weeks post induction animals were put into metabolic cages and 12 hour urine collections made and urinary albumin content assayed by ELISA. *p<0.05 compared with WT diabetic, ANOVA, Bonferroni. Blood glucose levels for STZ groups were similar STZ WT: 22.97±1.47 mmol/L vs STZ HET: 26.42±1.45 mmol/L (p=NS).

Discussion

The traditional view of the glomerular filtration barrier as a tri-layered filter has evolved significantly (41) with the identification of previously over looked ultra-structural (29, 30, 42) and biochemical (glycocalyx) (33) aspects of glomerular structure that contribute additional resistance to fluid and molecular flow, and with the realisation that GFB is more than a fixed passive sieve (or even a series of sieves) that provides resistance to the movement of water and solutes in a manner predicted by biophysical models. Overlying the complex ultra-structure are signalling pathways that are initiated within the GFB, and that serve to modify the GFB, and are required to maintain the normal glomerular phenotype. These signalling pathways appear to act across the GFB, and involve “crosstalk” between podocytes and adjacent GEC (12). Such cross-talk includes the VEGFₓₓₓ/VEGFₓₓₓb-VEGF-R2; VEGF-C-VEGF-R3 and Ang-1-Tie2 axes—all molecules that have been shown to affect microvascular permeability in other vascular beds (18, 2, 20, 24). These axes elicit paracrine alterations in the adjacent, but nevertheless “up-stream”, glomerular endothelium. The functional significance of these trans-GBM effects has been elegantly demonstrated by multiple podocyte specific transgenic models (14, 13, 11)—the phenotypes of some of which (proteinuria and glomerular thrombotic micro-angiopathy) (13) are reflected clinically in humans in the context of anti-VEGF therapy (eg the monoclonal antibody bevacizumab). These studies provide robust evidence that podocyte-derived VEGF is required to maintain GEC phenotype in the mature glomerulus. Overlying the endothelial cell changes resulting from podocyte VEGF derangement, is the fact that VEGF also undoubtedly has autocrine effects on podocytes themselves, this is true both for the VEGF₁₆₅ (16, 17), and VEGF₁₆₅b (5) isoforms, which, in the context of epithelial cell survival, have similar properties (16, 5).

It has been proposed then that podocyte-derived VEGF-A plays a crucial role in maintaining the filtration barrier through cell survival, proliferation and/or differentiation cues to the adjacent glomerular endothelium and to the podocytes themselves (14, 17). It is certainly an essential mediator of embryonic vasculogenesis since even heterozygous null VEGF-A mice die a few days post coitus (6). Its specific role in an established microvasculature such as the glomerulus is incompletely understood, however, multiple roles of glomerular VEGF and multiple VEGF isoforms with widely contrasting properties (22) perhaps goes someway to explaining the apparent experimental contradictions in the literature. In addition, the work of Eremina et al (14) was the first to support the notion that an optimal “dose of VEGF” in the glomerulus was likely to underpin the normal glomerular phenotype since podocyte specific transgenic over-expressing, or heterozygous KO mice, produce distinct glomerular phenotypes but both result in end stage kidney failure (14). Our study suggests that the “dose of VEGF” may include features that are qualitative (the balance of VEGFₓₓₓ/VEGFₓₓₓb isoforms) as well as quantitative (the absolute amounts of bio-available VEGF isoforms)Although difficult to make comprehensive direct comparisons of the KO-phenotype in the study of Eremina (14) (because the lox-P system will have knocked out VEGFₓₓₓ and VEGFₓₓₓb isoforms), the VEGF₁₆₄ podocyte-specific pnephrin-driven over-expressing transgenic animals are very comparable. This last produced a phenotype that showed ESRF secondary to collapsing nephropathy, a glomerular lesion typical of HIV-nephropathy (27). The kidney demonstrated renal haemorrhages and the animals died at day 5. This contrasts with our phenotype of modestly reduced permeability to water and urinary protein loss in animals that have a normal life expectancy. Our studies also show that the over-expression of VEGF₁₆₅b reduced the expression of constitutively expressed murine VEGF. This raises the question as to what proportion of the Eremina KO phenotypes are due to VEGFₓₓₓ inhibition and what degree of abnormalities resulted from VEGFₓₓₓb reduction. Furthermore, it is not clear what the contribution of murine VEGF plays in the model we present here. Future experiments on crosses between these two lines to assess the effectiveness of VEGFₓₓₓb in ameliorating the phenotype of the animals in Ereminas study may be informative as would isoform specific knockouts.

Although the podocyte-VEGF KO (14) are not directly comparable with our model, of note, the heterozygous animals in Eremina's study did demonstrate a loss of fenestrations (14). VEGF₁₆₅ has been shown to induce endothelial fenestrae ex vivo (38). The findings we present here suggest the qualitative balance of VEGF may be important for the establishment and maintenance of fenestrations in vivo.

VEGF expression in glomerulogenesis starts at the s-shape stage when a single capillary grows into the glomerular cleft. We have recently shown that at least some of the VEGF expression at that stage is VEGFₓₓₓb (5). The model described here is characterised by constitutive VEGF₁₆₅b over-expression, and therefore does not appear to influence the incoming migration of endothelial cells to the primitive glomerulus as the glomeruli in our appear histologically normal.

Using exon 8b VEGF specific ELISA we have shown that the exon 8b specific isoforms predominate in many tissues (3) and contribute to about half the VEGF in the normal kidney (5, 37). The contrast between the properties of these two families of isoforms is striking. Many laboratories worldwide have now confirmed, in receptor binding studies, in vitro endothelial proliferation and migration assays, ex vivo isolated resistance vessel myograph studies and in in vivo neo-vascular and tumour growth models, that VEGF₁₆₅b is not only not itself angiogenic but is actively anti-angiogenic (1, 48, 25, 7) inhibiting the action of VEGF₁₆₅. Thus, it reduces tumour growth of transfected melanoma (48), colon cancer (47), PC3 prostate cancer, Ewings Sarcoma, and CaKi renal carcinoma in nude mice (37). Furthermore, parenterally administered rhVEGF₁₆₅b (IP & SC) halts colonic carcinoma tumour growth in nude mice (46). We have also now shown that transgenic mice over-expressing VEGF₁₆₅b in mammary tissue have inhibited physiological angiogenesis (17). It is clear, therefore, that VEGF₁₆₅b can inhibit angiogenesis and vasodilatation, most likely through inhibition of VEGF₁₆₅ mediated activation of VEGF-R2.

In regard to micro-vessel permeability, studies using the Landis-Michel micro-occlusion technique, in cannulated single capillaries, have shown that in response to a bolus of rhVEGF₁₆₅b micro-vascular permeability to water increases for a few seconds only (rapidly returning to normal), apparently mediated by VEGF-R1 (20). However there is no physiological correlate to this and in the same study no chronic change in water permeability was seen in response to VEGF₁₆₅b, in contrast to that seen with VEGF₁₆₅ (21). VEGF₁₆₅b also inhibits VEGF₁₆₅-mediated reduction in Trans-endothelial monolayer resistance (TEER) (increased permeability) in vitro (5, 4).

The finding that exon 8a (eg VEGF₁₆₅) containing conventional isoforms tend to predominate in de-differentiated human conditionally immortalised podocytes that lack exon 8b-containing isoforms, the latter being present in differentiated podocytes, prompted Schumacher and colleagues to suggest that the maturation of podocytes (glomerular endothelial cells and hence the GBM) may depend on a ratio of these isoform families. In their study of VEGF-isoform family expression in Denys-Drash syndrome (DDS)(glomerular dysgenesis, FSGS leading to early onset nephritic syndrome and renal failure, and male pseudo-hermaphroditism) showed that although DDS podocytes produce ample pro-angiogenic, pro-permeability VEGF₁₆₅, they completely lack the anti-angiogenic, anti-permeability form VEGF₁₆₅b (43). The factors that influence splicing between the VEGF-A families are emerging (31) and since 74% human genes demonstrate mRNA splicing (44), it is perhaps no surprise that an increasing number of podocyte derived products display this property, eg the transcription factor WT-1 which has been implicated in Wilms tumours and DDS (28). WT-1 has 4 major isoforms. The zinc finger regions of WT-1 are able to bind both DNA and RNA and although the targets for WT-1 are unknown, as is its precise role, it has been shown that mutations in WT-1 in humans can lead to mesangial sclerosis and well characterised glomerular lesions (28, 36). It has been suggested that WT-1 might regulate the expression of factors that affect vascular development such as VEGF (28). WT-1 may therefore be one factor that controls the splicing of VEGF in podocytes and the associated glomerular phenotype.

Altered VEGF isoform balance has been potentially linked to other forms of glomerular lesion e.g. in a transgenic model in which the Hippel Lindau gene was deleted (10), leading to increased HIF-1α subunits, increased Cxcr4 expression and crescentic glomerulonephritis. In this model the podocytes were functionally responding to the signalling pathways activated in hypoxia which are known to increase VEGFₓₓₓ-expression but have no effect on VEGFₓₓₓb production (46).

In summary, here we show that VEGF₁₆₅b over-expression results in histologically, and physiologically healthy renal function, but with reduced glomerular permeability to water and urinary protein loss, and contrasts with VEGF₁₆₅ over-expression.

### EXAMPLE 3

Retinal Epithelial and Endothelial Cell Survival Studies

Materials and Methods

Human microvascular endothelial cells (HMVEC) were purchased from Cascade Biologics (Portland, Oreg., USA) and cultured in EGM-2MV media containing 5% FBS and supplements (Lonza Biologics, Switzerland). Human umbilical vein endothelial cells (HUVEC) were extracted from umbilical cords as previously described ¹(St Michael's Hospital, Bristol, UK) and cultured in EGM-MV2 media containing 5% FBS and supplements. Human retinal microvascular endothelial cells (REC) were purchased from Cell Systems (Kirkland, Wash., USA) and cultured in CSC Complete Media containing 10% FBS (Cell Systems, Kirkland, Wash., USA). Human retinal pigmented endothelial cells (RPE) were isolated from retinas of human eyes (Eye bank, Bristol, UK) and cultured in DMEM F12 containing 10% FBS (Gibco, Invitrogen, Paisley, UK). Immortalized ARPE-19 cells were purchased from ATCC and cultured in DMEM F12 media containing 10% FBS. Cells were confirmed by positivity by RT-PCR for cytokeratin 18, retinaldehyde binding protein 1 and retinol dehydrogenase 5.

VEGF-A₁₆₅b and VEGF-A₁₆₅

VEGF-A₁₆₅ protein was purchased from R&D, Minneapolis, Minn., USA and kindly provided by Kurt Ballmer-Hofer, Paul Scherrer Institute, Switzerland. PhiloGene Inc, Israel provided VEGF-A₁₆₅b protein.

Cytotoxicity Assay

Cells were seeded into 96 well plates (10,000 HUVEC and 15,000 ARPE-19), serum starved overnight and incubated in the presence or absence of H₂O₂, Na Butyrate or increasing concentrations of 7-ketocholesterol (Steraloids Inc., Newport, R.I.) and inhibitor or vehicle with or without 2.5 nM VEGF₁₆₅b {Rennel, 2008 #3849} (available on request from R&D systems, or Philogene Inc, New York). After 48 h (HUVEC) or 24 h (ARPE-19), 50 μl media was removed and cytotoxicity assayed using a lactate dehydrogenase (LDH) cytotoxicity detection kit (Promega, Madison, Wis., USA) and quantified using a Bichrometric Multiscan plate reader (Labsystems). To assay for total cell number, cells were lysed by the addition of 10 μl of 10× lysis buffer and the total level of LDH was assayed according to manufacturer's instructions. Cell viability assays (Cell Proliferation Reagent WST-1, Roche Diagnostics GmbH, Mannheim, Germany) on ARPE-19 cells were conducted according to the manufacturer's instructions. After 24 h test reagent incubation, 10 μl WST-1 reagent was added, plates were incubated at 37° C. and the ensuing colour development was quantified after 30 min and at hour intervals for 4 h using the aforementioned plate reader.

PCR on cDNA from Human Total RNA

1 mL of Trizol reagent was added to each well of a 6 well plate and mRNA extracted using the method of Chomczynski and Sacchi. 50% of the mRNA was reverse transcribed using MMLV RT, RNase H Minus, Point mutant (Promega) and polyd(T) (Promega) as a primer. Ten percent of the cDNA was then amplified using primers designed to detect VEGF and VEGFR2 (Table 2) and PCR Master Mix (Promega) were used in reactions cycled 30 times, denaturing at 95° C. for 60 seconds, annealing at 55° C. for 60 seconds and extending at 72° C. for 60 seconds. PCR products were run on agarose gels containing 0.5 μg/mL ethidium bromide and visualized under a UV transilluminator.

TABLE 2

| | | | PCR product |
|---|---|---|---|
| | Forward primer (5′-3′) | Reverse primer (5′-3′) | length (bp) |
| VEGFR2 | AAAACCTTTTGTTGCTT | GAAATGGGATTGGTA | 236 |
| | TGGA | AGATGA | |
| | (SEQ ID NO. 7) | (SEQ ID NO. 8) | |
| VEGFₓₓₓ/ | GGCAGCTTGAGTTAAA | ATGGATCCGTATCAG | (VEGF165) 123 |
| VEGFₓₓₓb | CGAACG | TCTTTCCTGC | (VEGF165b) 57 |
| | (SEQ ID NO. 9) | (SEQ ID NO. 10) | |

Trans Well Migration Assay

Endothelial cells for migration were used in passages 3-6 at 70-80% confluency. HMVECs were serum starved in endothelial basal media without FBS and supplements (EBM) for 8-10 h. Cells were trypsinized and re-suspended in 0.1% v/v FBS in EBM and 150000 cells in 500 μl medium were seeded on attachment factor (Cascade Biologics, Portland, Oreg., USA) coated filter inserts (8 μm, 12 mm, Millipore, Billerica, Mass., USA) with the treatment in the bottom well. Each treatment was performed in triplicate. The cells were incubated at 37° C. and allowed to migrate overnight. The inserts were washed with PBS and cells fixed with 4% PFA/PBS pH 7.4 for 10 min. Non-migrated cells were removed from the membranes and the nuclei of migrated cells stained with Hoechst 33258 (5 μg/ml in 0.5% Triton/PBS) and mounted on microscope slides with Vectashield (Vetorlabs, Burlingame, Calif., USA). Migrated cells were counted in 10 fields per membrane under the fluorescence microscope (Leica DM, Germany, 40× objective). The change in migration was expressed relatively to the basal migration rate towards zero chemo attractant and plotted as average±sem. The inhibitory effect on migration of VEGF-A-₁₆₅b over VEGF-A-₁₆₅ was determined by increasing concentrations of VEGF-A₁₆₅b (0-2 nM) with or without 1 nM VEGF-A₁₆₅. IC₅₀ was calculated from the normalized data using a variable slope sigmoidal fit (Prism4 software). RECs were serum starved and re-suspended for migration in CSC media without serum and growth factors (Cell Systems, Kirkland, Wash., USA) and the experiments performed as described above.

ICell Signalling

Serum starved human dermal endothelial cells were activated with 1 nM VEGF-A₁₆₅ or VEGF-A₁₆₅b. The cell lysates were run for protein separation on a 7.5% Laemmli acryl amide SDS gel under denaturing conditions. The proteins were blotted from the gel to a nitrocellulose membrane (wet transfer technique) and blocked with 5% BSA (Sigma-Aldrich, UK) in 0.05% Tween/PBS overnight at 4° C. The membranes were incubated first with either mouse anti-human-phospho-p38 MAP kinase (Thr180/Tyr182) antibody (9216), rabbit anti-human VEGF receptor 2 (Tyr1175), (2478), rabbit anti-human-VEGF receptor 2 antibody (2479), mouse-anti-human-phospho-p44/p42 MAPK (Thr202/Tyr204) antibody (9106), rabbit anti-human-p44/42 MAPK antibody (9102) (all from Cell Signalling Technologies) or mouse anti-human IGFBP3 antibody (Sigma, 2 μg/ml), in 3% BSA/0.05% Tween/PBS for 2.5 h at RT and secondary antibody 1:15,000 in 3% BSA/0.05% Tween/PBS for 45 min and processed as described above.

VEGF Protein Blotting

RPE cell lysates (30 μg total protein) and recombinant human control protein (30 ng) were run on a 12% Laemmli acryl amide SDS gel under denaturing conditions and processed as described above

Effect of VEGF on IGFBP3 Expression

Human primary RPE at passage 3-4 and at 70-80% confluency were cultured in serum free medium in the absence of FBS for 24 hours prior to treatment. Two ml of 1 ng/ml human recombinant VEGF-A₁₆₅ or VEGF-A₁₆₅b in serum-free medium were added. 24 hours later the RPE cells were washed 3 times with ice-cold PBS and lysed in 200 μl of Laemmli buffer for Western blotting as described above.

Cytotoxicity Effects of Antibodies on RPE Cells

A flask of sub-confluent freshly isolated RPE or ARPE-19 cells were seeded into 96 wells and grown until 70-80% confluent in 10% FBS DMEM F12. The media was changed to serum free DMEM and cells staved overnight before treated with mouse IgG, anti-VEGF-A₁₆₅b antibody (56/1) or bevacizumab (Avastin®) for 48hrs. Relative cell death was measured by the amount of lactase dehydrogenase (LDH) released into the culture media using a Cytotox Non-radioactive cytotoxicity assay (Promega, Madison, Wis., USA). The experiment was performed following the manufacturers protocol.

Immunohistochemistry on ARPE-19 Cells

Cells were grown on sterile cover slips until 50% confluent. Cells were fixed with 4% PFA/PBS pH 7.4 for 10min, washed with 2× PBS, blocked with 5% normal goat serum (Sigma-Aldrich, UK) in 0.5% triton/PBS for 1 h and incubated with anti-VEGF-A₁₆₅b antibody (R&DMAB3045) at 8 μg/ml in blocking solution overnight in a humidifying chamber. Cells were washed in 0.5% Triton/PBS and incubated with 1:400 goat anti-mouse AlexaFluor 546 antibody (Molecular Probes, Invtitrogen, UK) in blocking solution was applied for 3 hr and for the last 30 min a 1:200 dilution of phalloidin AlexaFluor 488 (Molecular Probes) and 5 μg/ml Hoechst 33258. After washing with triton/PBS, followed by PBS the cover slips were mounted with Vectashield and images were taken with the appropriate filters on a Leica DM fluorescence microscope (40× objective) and merged in Photoshop.

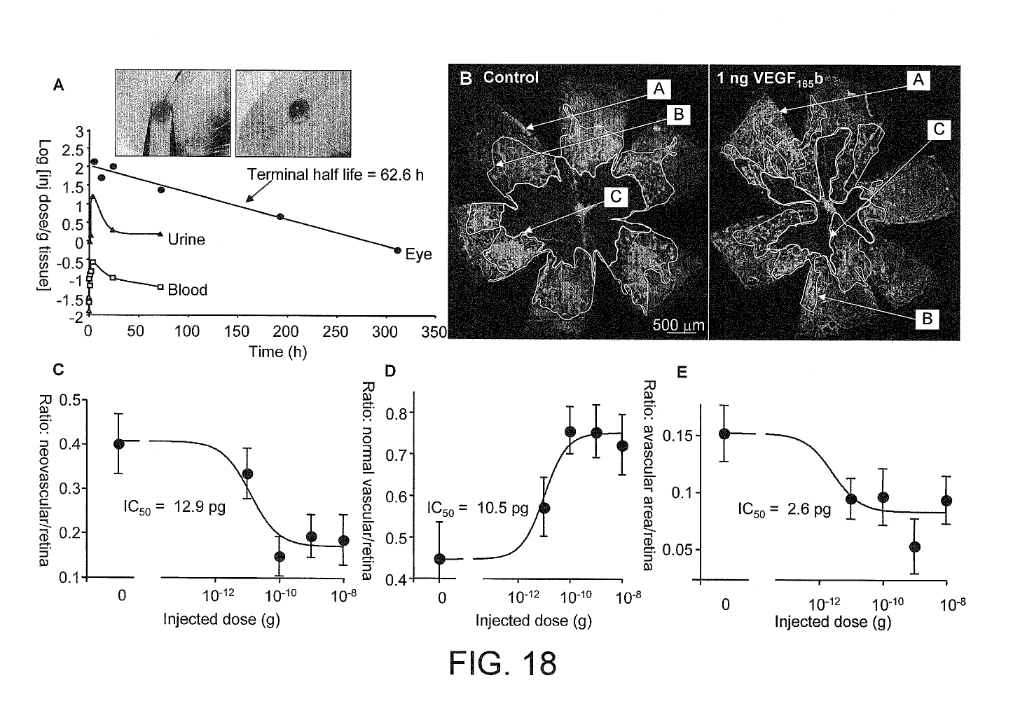

The results obtained are illustrated in FIGS. 18 to 22, which in particular illustrate the effect of VEGF₁₆₅b as an agent to support epithelial cell survival, and are discussed further below.

FIG. 18 illustrates that VEGF-A₁₆₅b inhibits neovascularization in the oxygen induced retinopathy model, but does not block revascularization.

As illustrated in FIG. 18A, intraocular injection of VEGF-A₁₆₅b has a half life of 62.6 h in the eye. ¹²⁵I-VEGF-A₁₆₅b was injected into the vitreous and the rats were culled and eyes, urine and blood were assayed using a gamma counter. The bi-exponential clearance was expressed as gamma counts per gram of tissue and the terminal half life is 2.6 days (62.6 h). Uptake into the urine and blood was seen within 30 min. Injection of fluorescein-dextran does not leak out after an intraocular injection of mice (inserted images).

As illustrated in FIG. 18B, mice were subjected to hyperoxia during postnatal development. Mice were injected with increasing concentrations of VEGF-A₁₆₅b or HBSS as a control and the retinal vessels visualised by isolectin B4 staining. The left image shows HBSS-(control) and the right VEGF-A₁₆₅b-treated retinas. The central ischemic avascular region (denoted by Arrow C), pre-retinal proliferation region (neovascularisation, denoted by Arrow B) and total vascularised retina (denoted by Arrow A) and the areas were measured.

As illustrated in FIG. 18C-E, the area in μm² of each defined region was measured in Image J. Neovascularization was significantly reduced by VEGF-A₁₆₅b injection (FIG. 18C), and the amount of normal vascularization was increased (FIG. 18D). This was partly a result of blood vessels growing into the avascular area reducing the avascular area (FIG. 18E). Thus VEGF-A₁₆₅b is able to maintain normal revascularization while inhibiting neovascularization, making it an ideal agent for ischemia induced angiogenesis.

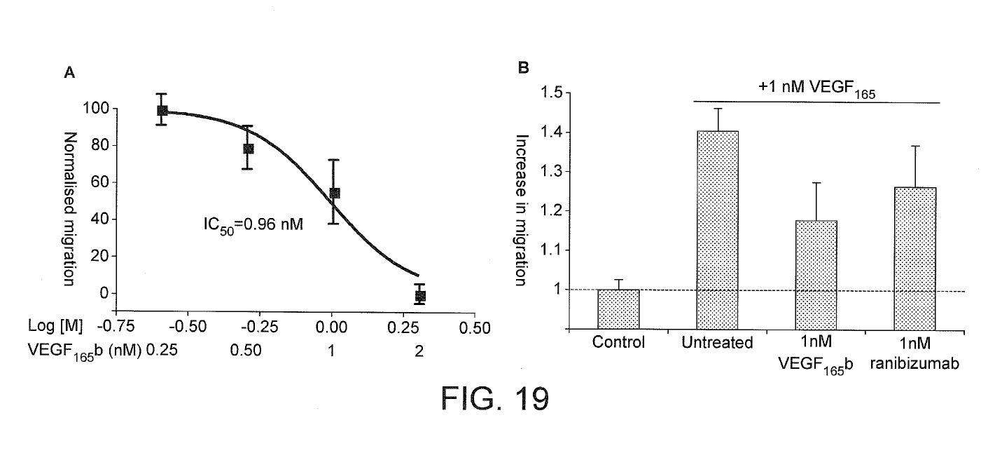

FIG. 19 illustrates that VEGF-A₁₆₅b inhibits human retinal endothelial cell migration.

As shown in FIG. 19A, human REC were seeded onto polycarbonate filters and migration towards increasing concentration of VEGF-A₁₆₅b was measured.

In FIG. 19B, inhibition of REC migration in response to 1 nM VEGF-A₁₆₅b was compared with 1 nM ranibizumab.

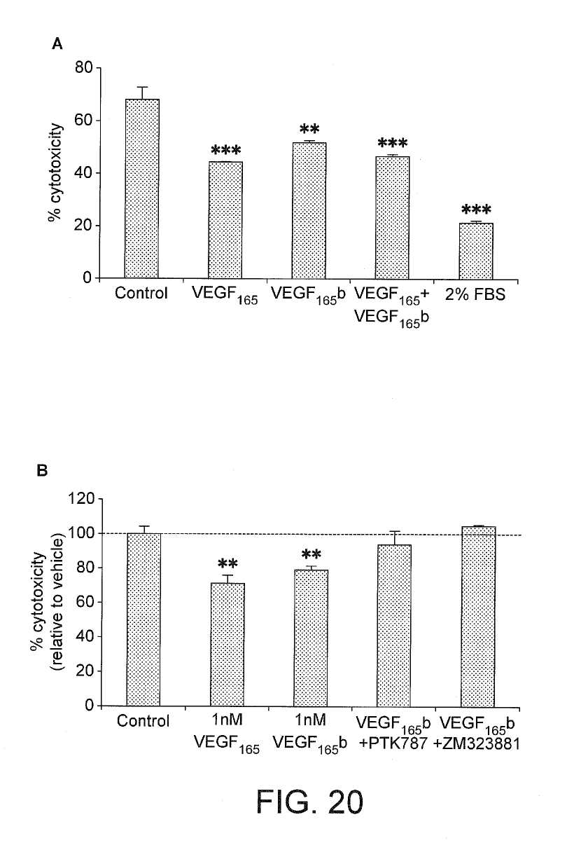

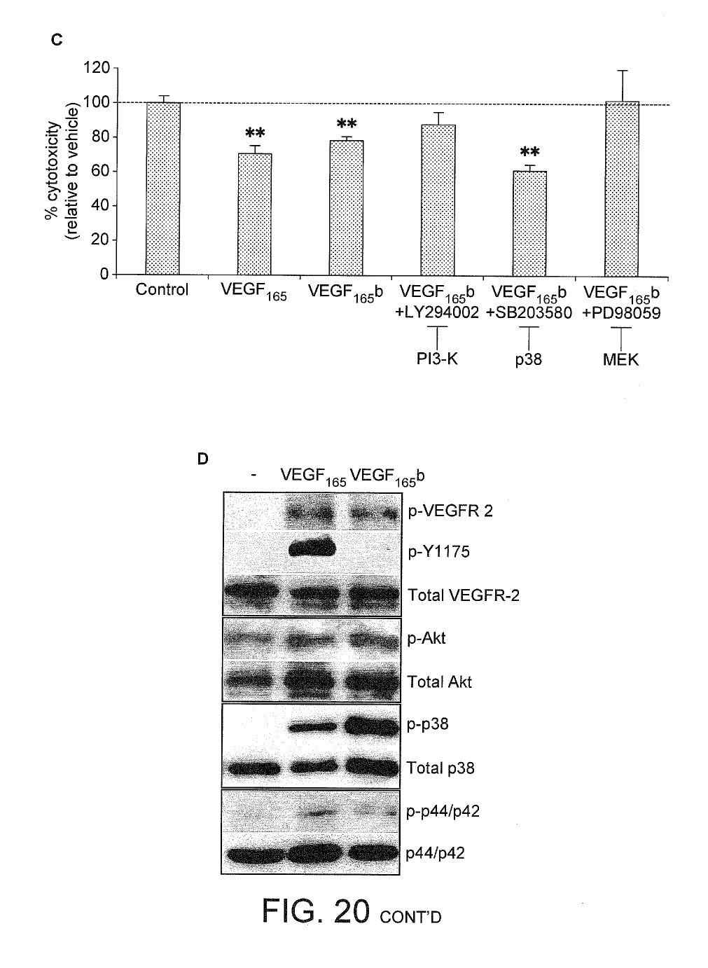

FIG. 20 illustrates that VEGF-A₁₆₅b is a survival factor for human endothelial cells.

In FIG. 20A, HUVEC cells were serum starved (0.1% serum, SFM). LDH assay to measure cytotoxicity after 48 h treatment with VEGF isoforms. VEGF-A₁₆₅ and VEGF-A₁₆₅b both inhibited cytotoxicity induced by serum starvation.

In FIG. 20B, cells were incubated either with VEGF-A₁₆₅b, VEGF-A₁₆₅, inhibitors, or VEGF-A₁₆₅b in the presence of VEGFR inhibitors and cytotoxicity measured by ELISA for LDH in the media. Cytotoxicity is expressed relative to the appropriate control (i.e. inhibitor in SFM). VEGFR inhibitors, PTK787 (blocks both VEGFR) and ZM323881 (specific to VEGFR2) inhibited the cytotoxicity.

In FIG. 20C, cells were treated with three different signal transduction inhibitors in the presence or absence of VEGF-A₁₆₅b, SB203580, which blocks p38MAPK, PD98059, which blocks p42/p44 MAPK phosphorylation by MEK, and LY294002, which inhibits PI3K and cytotoxicity measured. MEK and PI3K inhibitors blocked the reduction in cytotoxicity, but not p38MAPK inhibitor.

FIG. 20D illustrates activation of VEGFR2, Tyr residue 1175 of VEGFR2, Akt, p42p44MAPK, and p38MAPK in endothelial cells by VEGF-A₁₆₅ and VEGF-A₁₆₅b. Cells were treated for 10 min with VEGFs. **=p<0.01, ***=p<0.001, compared with control, one way ANOVA, Student Newman Keuls post hoc test.

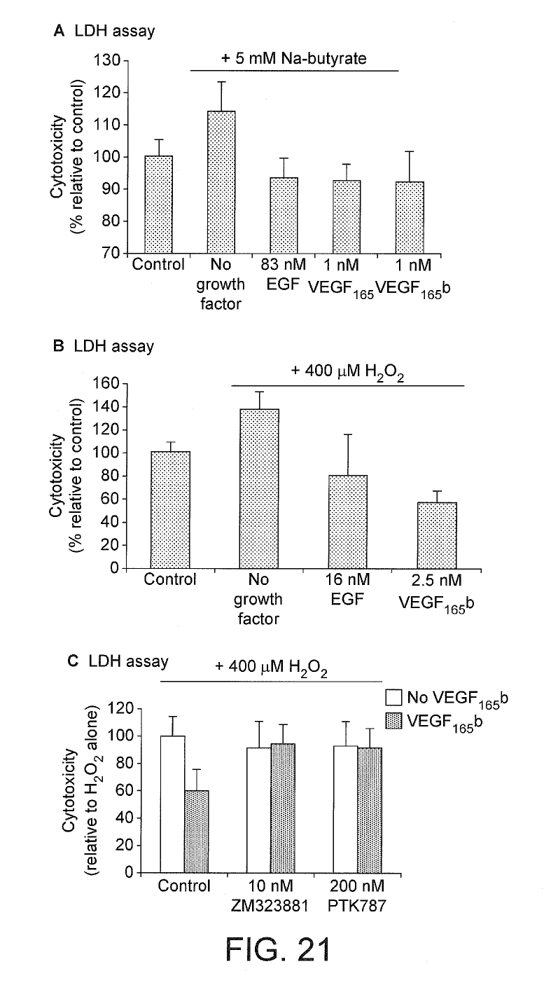

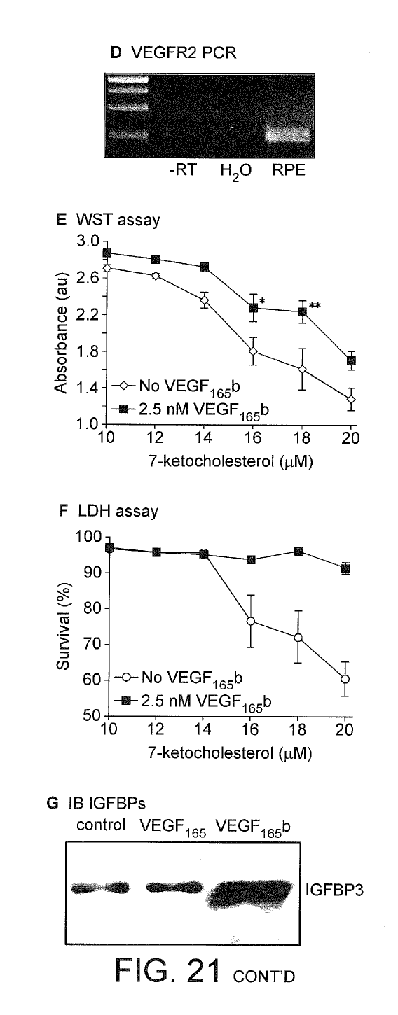

FIG. 21 illustrates that VEGF-A₁₆₅b is a cytoprotective agent for RPE cells.

In FIGS. 21A-C, ARPE19 cells were treated with either Na butyrate (FIG. 21A) or hydrogen peroxide (FIGS. 21B and 21C). Cells were incubated either with VEGF-A₁₆₅b, VEGF-A₁₆₅ or EGF and cytotoxicity measured by ELISA for LDH in the media. VEGF-A₁₆₅b inhibited cytotoxicity induced by Na Butyrate (FIG. 21A) and H₂O₂ (FIG. 21B). Cells were treated with H₂O₂ and two different inhibitors in the presence or absence of VEGF-A₁₆₅b (FIG. 21C). PTK787, which blocks both VEGFR1 and VEGFR2, or ZM323881, which is specific for VEGFR2. Both inhibitors blocked the reduction in cytotoxicity induced by VEGF-A₁₆₅b.

In FIG. 21D, RT-PCR of mRNA extracted from RPE cells indicate VEGFR2 expression.

In FIG. 21E, VEGF₁₆₅b reduced loss of cell viability induced by 7-ketocholesterol, as assessed by WST1 assay. Specifically, 2.5 nM VEGF165b increased ARPE-19 cell viability in the presence of 7-ketocholesterol compared to control as determined by WST-1 cell viability assay. 7-ketocholesterol treatment for 24 h and cells incubated with WST-1 for 240 min. The colour product (formazan salt) was read at 450 nm giving a measure of cell viability. VEGF165b does not induce ARPE-19 proliferation in treatment media indicating the observed increase in cell viability is due to cytoprotection.

In FIG. 21F, VEGF₁₆₅b reduced LDH release from cells during treatment with 7-ketocholesterol, and shows VEGF165b medieated cytoprotection.

In FIG. 21G, VEGF-A₁₆₅b increased IGFBP3 expression in RPE cells, whereas VEGF-A₁₆₅ did not.

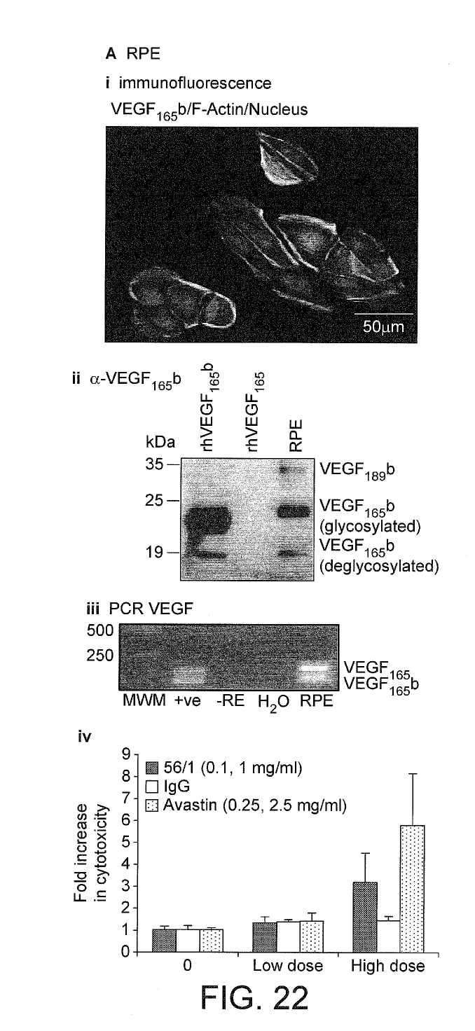

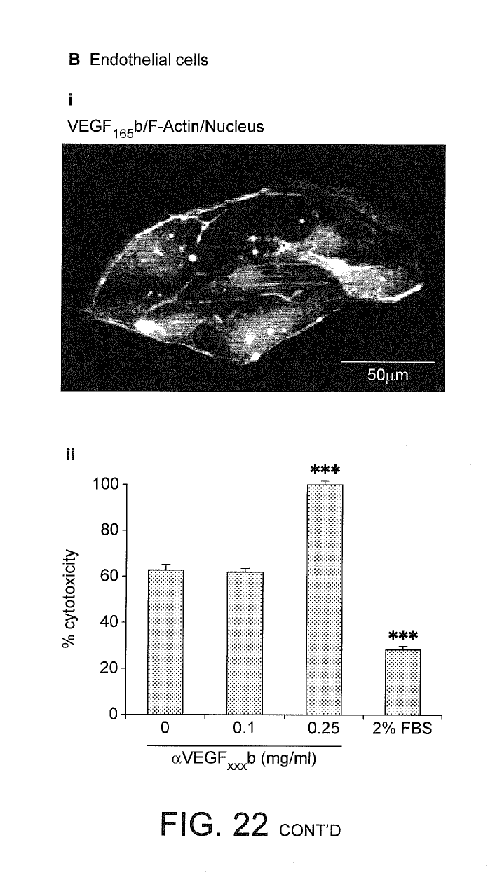

FIG. 22 illustrates that VEGF-A₁₆₅b is an endogenous survival factor.

In FIG. 22A, immunofluorescence staining revealed expression of VEGF₁₆₅b (red) in RPE cells (i) which was confirmed by western blotting (ii) using a VEGFₓₓₓb specific antibody, and mRNA confirmed by RT-PCR (iii). Inhibition of endogenous VEGFₓₓₓb or all VEGF isoforms by bevacizumab increased cytotoxicity (iv).

In FIG. 22B, human endothelial cells show VEGF₁₆₅b expression (i) and inhibition of VEGFₓₓₓb increased cytotoxicity (ii). ***=p<0.001 compared to control. Actin (green) and nucleus (blue).

### REFERENCES

Please note the existence below of separate lists of references, one of which is in square brackets [. . . ], another of which is in round brackets ( . . . ), and a third is a simple list of documents.

[1] Carmeliet P: Angiogenesis in health and disease. Nat Med 2003; 9:653-660.

[2] Robert B, Zhao X, Abrahamson D R: Coexpression of neuropilin-1, Flk1, and VEGF(164) in developing and mature mouse kidney glomeruli. Am J Physiol Renal Physiol 2000; 279:F275-282.

[3] Robert B, St John P L, Hyink D P, Abrahamson D R: Evidence that embryonic kidney cells expressing flk-1 are intrinsic, vasculogenic angioblasts. Am J Physiol 1996; 271:F744-753.

[4] Kitamoto Y, Tokunaga H, Tomita K: Vascular endothelial growth factor is an essential molecule for mouse kidney development: glomerulogenesis and nephrogenesis. J Clin Invest 1997; 99:2351-2357.

[5] Kaipainen A, Korhonen J, Pajusola K, Aprelikova O, Persico M G, Terman B I, Alitalo K: The related FLT4, FLT1, and KDR receptor tyrosine kinases show distinct expression patterns in human fetal endothelial cells. J Exp Med 1993; 178:2077-2088.

[6] Dumont D J, Fong G H, Puri M C, Gradwohl G, Alitalo K, Breitman M L: Vascularization of the mouse embryo: a study of flk-1, tek, tie, and vascular endothelial growth factor expression during development. Dev Dyn 1995; 203:80-92.

[7] Breier G, Albrecht U, Sterrer S, Risau W: Expression of vascular endothelial growth factor during embryonic angiogenesis and endothelial cell differentiation. Development 1992; 114:521-532.

[8] Eremina V, Sood M, Haigh J, Nagy A, Lajoie G, Ferrara N, Gerber H P, Kikkawa Y, Miner J H, Quaggin S E: Glomerular-specific alterations of VEGF-A expression lead to distinct congenital and acquired renal diseases. J Clin Invest 2003; 111:707-716.

[9] Eremina V, Quaggin S E: The role of VEGF-A in glomerular development and function. Curr Opin Nephrol Hypertens 2004; 13:9-15.

[10] Gerber H P, Hillan K J, Ryan A M, Kowalski J, Keller G A, Rangell L, Wright B D, Radtke F, Aguet M, Ferrara N: VEGF is required for growth and survival in neonatal mice. Development 1999; 126:1149-1159.

[11] Schrijvers B F, Flyvbjerg A, De Vriese A S: The role of vascular endothelial growth factor (VEGF) in renal pathophysiology. Kidney Int 2004; 65:2003-2017.

[12] Foster R R, Hole R, Anderson K, Satchell S C, Coward R J, Mathieson P W, Gillatt D A, Saleem M A, Bates D O, Harper S J: Functional evidence that vascular endothelial growth factor may act as an autocrine factor on human podocytes. Am J Physiol Renal Physiol 2003; 284:F1263-1273.

[13] Houck K A, Ferrara N, Winer J, Cachianes G, Li B, Leung D W: The vascular endothelial growth factor family: identification of a fourth molecular species and characterization of alternative splicing of RNA. Mol Endocrinol 1991; 5:1806-1814.

[14] Bates D O, Cui T G, Doughty J M, Winkler M, Sugiono M, Shields J D, Peat D, Gillatt D, Harper S J: VEGF165b, an inhibitory splice variant of vascular endothelial growth factor, is down-regulated in renal cell carcinoma. Cancer Res 2002; 62:4123-4131.

[15] Perrin R M, Konopatskaya O, Qiu Y, Harper S, Bates D O, Churchill A J: Diabetic retinopathy is associated with a switch in splicing from anti- to pro-angiogenic isoforms of vascular endothelial growth factor. Diabetologia 2005; 48:2422-2427.

[16] Woolard J, Wang W Y, Bevan H S, Qiu Y, Morbidelli L, Pritchard-Jones R O, Cui T G, Sugiono M, Waine E, Perrin R, Foster R, Digby-Bell J, Shields J D, Whittles C E, Mushens R E, Gillatt D A, Ziche M, Harper S J, Bates D O: VEGF165b, an inhibitory vascular endothelial growth factor splice variant: mechanism of action, in vivo effect on angiogenesis and endogenous protein expression. Cancer Res 2004; 64:7822-7835.

[17] Konopatskaya O, Churchill A J, Harper S J, Bates D O, Gardiner T A: VEGF₁₆₅b, an endogenous C-terminal splice variant of VEGF, inhibits retinal neovascularisation in mice. Mol Vis 2006, 12, 626-32.

[18] Cebe Suarez S, Pieren M, Cariolato L, Am S, Hoffmann U, Bogucki A, Manlius C, Wood J, Ballmer-Hofer K: A VEGF-A splice variant defective for heparan sulfate and neuropilin-1 binding shows attenuated signaling through VEGFR-2. Cell Mol Life Sci 2006; 63:2067-2077.

[19] Glass C A, Harper S J, Bates D O: The anti-angiogenic VEGF isoform VEGF165b transiently increases hydraulic conductivity, probably through VEGF receptor 1 in vivo. J Physiol 2006; 572:243-257.

[20] Schumacher V, Jeruschke S, Eitner F, Becker J, Pitschke G, Ince C, Miner J H, I Leuschner, R Engers, A S Everding, M Bulla, Royer-Pokora B: Impaired glomerular maturation and lack of VEGF165b in Denys-Drash syndrome. J Am Soc Nephrol 2007, 18, 719-29.

[21] Varey A H, Rennel E S, Qiu Y, Bevan H S, Perrin R M, Raffy S, Dixon A R, Paraskeva C, Zaccheo O, Hassan A B, Harper S J, Bates D O: VEGF 165 b, an antiangiogenic VEGF-A isoform, binds and inhibits bevacizumab treatment in experimental colorectal carcinoma: balance of pro- and antiangiogenic VEGF-A isoforms has implications for therapy. Br J Cancer 2008; 98:1366-1379.

[22] Foster R R, Saleem M A, Mathieson P W, Bates D O, Harper S J: Vascular endothelial growth factor and nephrin interact and reduce apoptosis in human podocytes. Am J Physiol Renal Physiol 2005; 288:F48-57.

[23] Rennel E S, H-Zadeh M A, Wheatley E, Schuler Y, Kelly S P, Cebe Suarez S, Ballmer-Hofer K, Stewart L, Bates D O, Harper S J: Recombinant human VEGF₁₆₅b protein is an effective anti-cancer agent in mice. Eur J Cancer 2008; 44:1883-1894.

[24] Satchell S C, Anderson K L, Mathieson P W: Angiopoietin 1 and vascular endothelial growth factor modulate human glomerular endothelial cell barrier properties. J Am Soc Nephrol 2004; 15:566-574.

[25] Bates D O, Macmillan P P, Manjaly J G, Qiu Y, Hudson S J, Bevan H S, Hunter A J, Soothill P W, Read M, Donaldson L F, Harper S J: The endogenous anti-angiogenic family of splice variants of VEGF, VEGF xxxb, are down-regulated in pre-eclamptic placentae at term. Clin Sci (Lund) 2006.

[26] Cui T G, Foster R R, Saleem M, Mathieson P W, Gillatt D A, Bates D O, Harper S J: Differentiated human podocytes endogenously express an inhibitory isoform of vascular endothelial growth factor (VEGF165b) mRNA and protein. Am J Physiol Renal Physiol 2004; 286:F767-773.

[27] Simon M, Rockl W, Hornig C, Grone E F, Theis H, Weich H A, Fuchs E, Yayon A, Grone H J: Receptors of vascular endothelial growth factor/vascular permeability factor (VEGF/VPF) in fetal and adult human kidney: localization and [1251]VEGF binding sites. J Am Soc Nephrol 1998; 9:1032-1044.

[28] Tufro A, Norwood V F, Carey R M, Gomez R A: Vascular endothelial growth factor induces nephrogenesis and vasculogenesis. J Am Soc Nephrol 1999; 10:2125-2134.

[29] Simon M, Grone H J, Johren O, Kullmer J, Plate K H, Risau W, Fuchs E: Expression of vascular endothelial growth factor and its receptors in human renal ontogenesis and in adult kidney. Am J Physiol 1995; 268:F240-250.

[30] Bailey E, Bottomley M J, Westwell S, Pringle J H, Furness P N, Feehally J, Brenchley P E, Harper S J: Vascular endothelial growth factor mRNA expression in minimal change, membranous, and diabetic nephropathy demonstrated by non-isotopic in situ hybridisation. J Clin Pathol 1999; 52:735-738.

[31] Sadl V, Jin F, Yu J, Cui S, Holmyard D, Quaggin S, Barsh G, Cordes S: The mouse Kreisler (Krml1/MafB) segmentation gene is required for differentiation of glomerular visceral epithelial cells. Dev Biol 2002; 249:16-29.

[32] Loughna S, Hardman P, Landels E, Jussila L, Alitalo K, Woolf A S: A molecular and genetic analysis of renalglomerular capillary development. Angiogenesis 1997; 1:84-101.

[33] Takemoto M, He L, Norlin J, Patrakka J, Xiao Z, Petrova T, Bondjers C, Asp J, Wallgard E, Sun Y, Samuelsson T, Mostad P, Lundin S, Miura N, Sado Y, Alitalo K, Quaggin S E, Tryggvason K, Betsholtz C: Large-scale identification of genes implicated in kidney glomerulus development and function. Embo J 2006; 25:1160-1174.

[34] Wong M A, Cui S, Quaggin S E: Identification and characterization of a glomerular-specific promoter from the human nephrin gene. Am J Physiol Renal Physiol 2000; 279:F1027-1032.

[35] Robert B, Abrahamson D R: Control of glomerular capillary development by growth factor/receptor kinases. Pediatr Nephrol 2001; 16:294-301.

[36] Dolan V, Hensey C, Brady H R: Diabetic nephropathy: renal development gone awry? Pediatr Nephrol 2003; 18:75-84.

[37] Bernhardt W M, Schmitt R, Rosenberger C, Munchenhagen P M, Grone H J, Frei U, Warnecke C, Bachmann S, Wiesener M S, Willam C, Eckardt K U: Expression of hypoxia-inducible transcription factors in developing human and rat kidneys. Kidney Int 2006; 69:114-122.

[38] Carmeliet P, Ferreira V, Breier G, Pollefeyt S, Kieckens L, Gertsenstein M, Fahrig M, Vandenhoeck A, Harpal K, Eberhardt C, Declercq C, Pawling J, Moons L, Collen D, Risau W, Nagy A: Abnormal blood vessel development and lethality in embryos lacking a single VEGF allele. Nature 1996; 380:435-439.

[39] Ferrara N, Carver-Moore K, Chen H, Dowd M, Lu L, O'Shea K S, Powell-Braxton L, Hillan K J, Moore M W: Heterozygous embryonic lethality induced by targeted inactivation of the VEGF gene. Nature 1996; 380:439-442.

[40] Ferguson J, Qiu Y, Sage L M, Neal C R, Bates D O, Harper S J, Salmon A H: Ultrafiltration co-efficient in isolated intact glomeruli from podocyte specific vegf₁₆₅b over-expressing transgenic mice. Microcirculation 2007; 14.

[41] Amin E M, Nowak D G, Saleem M, Bates D, Ladomery M: Relationship between wt1 and vascular endothelial growth factor (vegf) splicing. Microcirculation 2007; 14.

[42] Holash J, Davis S, Papadopoulos N, Croll S D, Ho L, Russell M, Boland P, Leidich R, Hylton D, Burova E, Ioffe E, Huang T, Radziejewski C, Bailey K, Fandl J P, Daly T, Wiegand S J, Yancopoulos G D, Rudge J S: VEGF-Trap: a VEGF blocker with potent antitumor effects. Proc Natl Acad Sci USA 2002; 99:11393-11398.

[43] Kendall R L, Wang G, Thomas K A: Identification of a natural soluble form of the vascular endothelial growth factor receptor, FLT-1, and its heterodimerization with KDR. Biochem Biophys Res Commun 1996; 226:324-328.

[44] Presta L G, Chen H, O'Connor S J, Chisholm V, Meng Y G, Krummen L, Winkler M, Ferrara N: Humanization of an anti-vascular endothelial growth factor monoclonal antibody for the therapy of solid tumors and other disorders. Cancer Res 1997; 57:4593-4599.

[45] Belteki G, Haigh J, Kabacs N, Haigh K, Sison K, Costantini F, Whitsett J, Quaggin S E, Nagy A: Conditional and inducible transgene expression in mice through the combinatorial use of Cre-mediated recombination and tetracycline induction. Nucleic Acids Res 2005; 33:e51.

[46] Quaggin S E: Transcriptional regulation of podocyte specification and differentiation. Microsc Res Tech 2002; 57:208-211.

(1) Bates, D O, Cui, TG, Doughty, J M, Winkler, M, Sugiono, M, Shields, J D, Peat, D, Gillatt, D & Harper, S J: VEGF165b, an inhibitory splice variant of vascular endothelial growth factor, is down-regulated in renal cell carcinoma. Cancer Research, 62: 4123-31., 2002.

(2) Bates, D O & Curry, F E: Vascular endothelial growth factor increases hydraulic conductivity of isolated perfused microvessels. American Journal of Physiology: Heart and Circulatory Physiology, 271: H2520-H2528, 1996.

(3) Bevan, H S, Harper, S. J., Bates, D. O.: Vascular Endothelial Growth Factors. In: Angiogenesis: Basic Science and Clinical Applications. ISBN:978-81-7895-302-1. edited by MARAGOUDAKIS, M. E., PAPADIMITRIOU, E., Kerala, Transworld Research Network, 2007, pp pp 1-26.

(4) Bevan, H S, van den Akker, N M, Qiu, Y, Polman, J A, Foster, R R, Yem, J, Nishikawa, A, Satchell, S C, Harper, S J, Gittenberger de Groot, A C & Bates, D O: The alternatively spliced anti-angiogenic family of VEGF isoforms VEGFxxxb in human kidney development. Nephron Physiol, 110: p57-67, 2008.

(5) Bevan, H S, van den Akker, N M, Qiu, Y, Polman, J A, Foster, R R, Yem, J, Nishikawa, A, Satchell, S C, Harper, S J, Gittenberger-de Groot, A C & Bates, D O: The alternatively spliced anti-angiogenic family of VEGF isoforms VEGFxxxb in human kidney development. Nephron Physiol, 110: p57-67, 2008.

(6) Carmeliet, P, Ferreira, V, Breier, G, Pollefeyt, S, Kieckens, L, Gertsenstein, M, Fahrig, M, Vandenhoeck, A, Harpal, K, Eberhardt, C, Declercq, C, Pawling, J, Moons, L, Collen, D, Risau, W & Nagy, A: Abnormal blood vessel development and lethality in embryos lacking a single VEGF allele. Nature, 380: 435-9, 1996.

(7) Cebe Suarez, S, Pieren, M, Cariolato, L, Arn, S, Hoffmann, U, Bogucki, A, Manlius, C, Wood, J & Ballmer-Hofer, K: A VEGF-A splice variant defective for heparan sulfate and neuropilin-1 binding shows attenuated signaling through VEGFR-2. Cell Mol Life Sci, 63: 2067-77, 2006.

(8) Chobanian, A V, Bakris, G L, Black, H R, Cushman, W C, Green, L A, Izzo, J L, Jr., Jones, D W, Materson, B J, Oparil, S, Wright, J T, Jr. & Roccella, E J: The Seventh Report of the Joint National Committee on Prevention, Detection, Evaluation, and Treatment of High Blood Pressure: the JNC 7 report. JAMA, 289: 2560-72, 2003.

(9) de Vriese, A: Antibodies against vascular endothelial growth factor improving early renal dysfunction in experimental diabetes. J Am Soc Nephrol, 12: 993-1000, 2001.

(10) Ding, M: Loss of the tumor suppressor Vhlh leads to upregulation of Cxcr4 and rapidly progressive glomerulonephritis in mice. Nat Medicine, 12: 1081-1087, 2006.

(11) Ding, M, Cui, S, Li, C, Jothy, S, Haase, V, Steer, B M, Marsden, P A, Pippin, J, Shankland, S, Rastaldi, M P, Cohen, C D, Kretzler, M & Quaggin, S E: Loss of the tumor suppressor Vhlh leads to upregulation of Cxcr4 and rapidly progressive glomerulonephritis in mice. Nat Med, 12: 1081-7, 2006.

(12) Eremina, V, Baelde, H J & Quaggin, S E: Role of the VEGF—a signaling pathway in the glomerulus: evidence for crosstalk between components of the glomerular filtration barrier. Nephron Physiol, 106: p32-7, 2007.

(13) Eremina, V, Jefferson, J A, Kowalewska, J, Hochster, H, Haas, M, Weisstuch, J, Richardson, C, Kopp, J B, Kabir, M G, Backx, P H, Gerber, H P, Ferrara, N, Barisoni, L, Alpers, C E & Quaggin, S E: VEGF inhibition and renal thrombotic microangiopathy. N Engl J Med, 358: 1129-36, 2008.

(14) Eremina, V, Sood, M, Haigh, J, Nagy, A, Lajoie, G, Ferrara, N, Gerber, H P, Kikkawa, Y, Miner, J H & Quaggin, S E: Glomerular-specific alterations of VEGF-A expression lead to distinct congenital and acquired renal diseases. J Clin Invest, 111: 707-16, 2003.

(15) Feng, D, Nagy, J A, Brekken, R A, Pettersson, A, Manseau, E J, Pyne, K, Mulligan, R, Thorpe, P E, Dvorak, H F & Dvorak, A M: Ultrastructural localization of the vascular permeability factor/vascular endothelial growth factor (VPF/VEGF) receptor-2 (FLK-1, KDR) in normal mouse kidney and in the hyperpermeable vessels induced by VPF/VEGF-expressing tumors and adenoviral vectors. J Histochem Cytochem, 48: 545-56, 2000.

(16) Foster, R R, Hole, R, Anderson, K, Satchell, S C, Coward, R J, Mathieson, P W, Gillatt, D A, Saleem, M A, Bates, D O & Harper, S J: Functional evidence that vascular endothelial growth factor may act as an autocrine factor on human podocytes. Am J Physiol Renal Physiol, 284: F1263-73, 2003.

(17) Foster, R R, Saleem, M A, Mathieson, P W, Bates, D O & Harper, S J: Vascular endothelial growth factor and nephrin interact and reduce apoptosis in human podocytes. Am J Physiol Renal Physiol, 288: F48-57, 2005.

(18) Gavard, J, Patel, V & Gutkind, J S: Angiopoietin-1 prevents VEGF-induced endothelial permeability by sequestering Src through mDia. Dev Cell, 14: 25-36, 2008.

(19) Gerstein, H C, Mann, J F, Yi, Q, Zinman, B, Dinneen, S F, Hoogwerf, B, Halle, J P, Young, J, Rashkow, A, Joyce, C, Nawaz, S & Yusuf, S: Albuminuria and risk of cardiovascular events, death, and heart failure in diabetic and nondiabetic individuals. JAMA, 286: 421-6, 2001.

(20) Glass, C A, Harper, S J & Bates, D O: The anti-angiogenic VEGF isoform VEGF165b transiently increases hydraulic conductivity, probably through VEGF receptor 1 in vivo. Journal of Physiology, 572: 243-57, 2006.

(21) Glass, C A, Harper, S J & Bates, D O: The anti-angiogenic VEGF isoform VEGF165b transiently increases hydraulic conductivity, probably through VEGF receptor 1 in vivo. J Physiol, 572: 243-57, 2006.

(22) Harper, S J & Bates, D O: VEGF-A splicing: the key to anti-angiogenic therapeutics? Nat Rev Cancer, 8: 880-7, 2008.

(23) Hayat, M A: Chemical Fixation In: Principles and Techniques of Electron Microscopy. edited by Hayat, M., London, Macmillan, 1989, pp 311-315.

(24) Hillman, N J, Whittles, C E, Pocock, T M, Williams, B & Bates, D O: Differential effects of vascular endothelial growth factor-C and placental growth factor-1 on the hydraulic conductivity of frog mesenteric capillaries. J Vasc Res, 38: 176-86, 2001.

(25) Konopatskaya, O, Churchill, A J, Harper, S J, Bates, D O & Gardiner, T A: VEGF165b, an endogenous C-terminal splice variant of VEGF, inhibits retinal neovascularization in mice. Mol Vis, 12: 626-32, 2006.

(26) Lambrechts, D, Storkebaum, E, Morimoto, M, Del-Favero, J, Desmet, F, Marklund, S L, Wyns, S, Thijs, V, Andersson, J, van Marion, I, Al-Chalabi, A, Bornes, S, Musson, R, Hansen, V, Beckman, L, Adolfsson, R, Pall, H S, Prats, H, Vermeire, S, Rutgeerts, P, Katayama, S, Awata, T, Leigh, N, Lang-Lazdunski, L, Dewerchin, M, Shaw, C, Moons, L, Vlietinck, R, Morrison, K E, Robberecht, W, Van Broeckhoven, C, Collen, D, Andersen, P M & Carmeliet, P: VEGF is a modifier of amyotrophic lateral sclerosis in mice and humans and protects motoneurons against ischemic death. Nat Genet, 34: 383-94, 2003.

(27) Laurinavicius, A, Hurwitz, S & Rennke, H G: Collapsing glomerulopathy in HIV and non-HIV patients: a clinicopathological and follow-up study. Kidney Int, 56: 2203-13, 1999.

(28) Morrison, A A, Viney, R L, Saleem, M A & Ladomery, M R: New insights into the function of the Wilms tumor suppressor gene WT1 in podocytes. Am J Physiol Renal Physiol, 295: F12-7, 2008.

(29) Neal, C R, Crook, H, Bell, E, Harper, S J & Bates, D O: Three-dimensional reconstruction of glomeruli by electron microscopy reveals a distinct restrictive urinary subpodocyte space. J Am Soc Nephrol, 16: 1223-35, 2005.

(30) Neal, C R, Muston, P R, Njegovan, D, Verrill, R, Harper, S J, Deen, W M & Bates, D O: Glomerular filtration into the subpodocyte space is highly restricted under physiological perfusion conditions. Am J Physiol Renal Physiol, 293: F1787-98, 2007.

(31) Nowak, D G, et al Expression of pro- and anti-angiogenic isoforms of VEGF is differentially regulated by known splicing and growth factors. J Cell Sci, 121, 3487-3495, 2008.

(32) Ostendorf, T, Kunter, U, Eitner, F, Loos, A, Regele, H, Kerjaschki, D, Henninger, D D, Janjic, N & Floege, J: VEGF(165) mediates glomerular endothelial repair. J Clin Invest, 104: 913-23, 1999.

(33) Perrin, R M, Harper, S J & Bates, D O: A role for the endothelial glycocalyx in regulating microvascular permeability in diabetes mellitus. Cell Biochem Biophys, 49: 65-72, 2007.

(34) Pinto-Sietsma, S J, Janssen, W M, Hillege, H L, Navis, G, De Zeeuw, D & De Jong, P E: Urinary albumin excretion is associated with renal functional abnormalities in a nondiabetic population. J Am Soc Nephrol, 11: 1882-8, 2000.

(35) Qiu, Y, Bevan, H, Weeraperuma, S, Wratting, D, Murphy, D, Neal, C R, Bates, D O & Harper, S J: Mammary alveolar development during lactation is inhibited by the endogenous antiangiogenic growth factor isoform, VEGF165b. FASEB J, 22: 1104-12, 2008.

(36) Quaggin, S E, Kreidberg, J. A.: Developemnt of the renal glomerulus: ggod neighbors and good fences. Development, 135: 609-620, 2008.

(37) Rennel, E S, Waine, E, Guan, H, Schuler, Y, Leenders, W, Woolard, J, Sugiono, M, Gillatt, D, Kleinerman, E S, Bates, D O & Harper, S J: The endogenous anti-angiogenic VEGF isoform, VEGF(165)b inhibits human tumour growth in mice. Br J Cancer, 98: 1250-7, 2008.

(38) Roberts, W G & Palade, G E: Increased microvascular permeability and endothelial fenestration induced by vascular endothelial growth factor. J Cell Sci, 108 (Pt 6): 2369-79, 1995.

(39) Saleem, M A, O'Hare, M J, Reiser, J, Coward, R J, Inward, C D, Farren, T, Xing, C Y, Ni, L, Mathieson, P W & Mundel, P: A conditionally immortalized human podocyte cell line demonstrating nephrin and podocin expression. J Am Soc Nephrol, 13: 630-8, 2002.

(40) Salmon, A H, Neal, C R, Bates, D O & Harper, S J: Vascular endothelial growth factor increases the ultrafiltration coefficient in isolated intact Wistar rat glomeruli. J Physiol, 570: 141-56, 2006.

(41) Salmon, A H, Neal, C. R., Harper, S. J.: New Aspects of Glomerular Filtration Barrier Structure and Function −5 layers (at least) not 3. Curr Opin Nephrol Hypertens, 2009 (In Press).

(42) Salmon, A H, Toma, I, Sipos, A, Muston, P R, Harper, S J, Bates, D O, Neal, C R & Peti-Peterdi, J: Evidence for restriction of fluid and solute movement across the glomerular capillary wall by the subpodocyte space. Am J Physiol Renal Physiol, 293: F1777-86, 2007.

(43) Schumacher, V A, Jeruschke, S, Eitner, F, Becker, J U, Pitschke, G, Ince, Y, Miner, J H, Leuschner, I, Engers, R, Everding, A S, Bulla, M & Royer-Pokora, B: Impaired glomerular maturation and lack of VEGF165b in Denys-Drash syndrome. J Am Soc Nephrol, 18: 719-29, 2007.

(44) Smith, C W & Valcarcel, J: Alternative pre-mRNA splicing: the logic of combinatorial control. Trends Biochem Sci, 25: 381-8, 2000.

(45) Sugimoto, H, Hamano, Y, Charytan, D, Cosgrove, D, Kieran, M, Sudhakar, A & Kalluri, R: Neutralization of circulating vascular endothelial growth factor (VEGF) by anti-VEGF antibodies and soluble VEGF receptor 1 (sFlt-1) induces proteinuria. J Biol Chem, 278: 12605-8, 2003.

(46) Varey, A H, Rennel, E S, Qiu, Y, Bevan, H S, Perrin, R M, Raffy, S, Dixon, A R, Paraskeva, C, Zaccheo, O, Hassan, A B, Harper, S J & Bates, D O: VEGF(165)b, an antiangiogenic VEGF-A isoform, binds and inhibits bevacizumab treatment in experimental colorectal carcinoma: balance of pro- and antiangiogenic VEGF-A isoforms has implications for therapy. Br J Cancer, 98: 1366-1379, 2008.

(47) Varey, A H, Rennel, E S, Qiu, Y, Bevan, H S, Perrin, R M, Raffy, S, Dixon, A R, Paraskeva, C, Zaccheo, O, Hassan, A B, Harper, S J & Bates, D O: VEGF(165)b, an antiangiogenic VEGF-A isoform, binds and inhibits bevacizumab treatment in experimental colorectal carcinoma: balance of pro- and antiangiogenic VEGF-A isoforms has implications for therapy. Br. J. Cancer, 98(8): 1366-1379, 2008.

(48) Woolard, J, Wang, W Y, Bevan, H S, Qiu, Y, Morbidelli, L, Pritchard-Jones, R O, Cui, T G, Sugiono, M, Waine, E, Perrin, R, Foster, R, Digby-Bell, J, Shields, J D, Whittles, C E, Mushens, R E, Gillatt, D A, Ziche, M, Harper, S J & Bates, D O: VEGF165b, an inhibitory vascular endothelial growth factor splice variant: mechanism of action, in vivo effect on angiogenesis and endogenous protein expression. Cancer Res, 64: 7822-35, 2004.

(49) Yang, J C, Haworth, L, Sherry, R M, Hwu, P, Schwartzentruber, D J, Topalian, S L, Steinberg, S M, Chen, H X & Rosenberg, S A: A randomized trial of bevacizumab, an anti-vascular endothelial growth factor antibody, for metastatic renal cancer. N Engl J Med, 349: 427-34, 2003.

### FURTHER REFERENCES

Davis, B, Dei Cas, A, Long, D A, White, K E, Hayward, A, Ku, C H, Woolf, A S, Bilous, R, Viberti, G & Gnudi, L: Podocyte-specific expression of angiopoietin-2 causes proteinuria and apoptosis of glomerular endothelia. J Am Soc Nephrol, 18: 2320-9, 2007.

Ichimura, K, Stan, R V, Kurihara, H & Sakai, T: Glomerular endothelial cells form diaphragms during development and pathologic conditions. J Am Soc Nephrol, 19: 1463-71, 2008.

Kamba, T, Tam, B Y, Hashizume, H, Haskell, A, Sennino, B, Mancuso, M R, Norberg, S M, O'Brien, S M, Davis, R B, Gowen, L C, Anderson, K D, Thurston, G, Joho, S, Springer, M L, Kuo, C J & McDonald, D M: VEGF-dependent plasticity of fenestrated capillaries in the normal adult microvasculature. Am J Physiol Heart Circ Physiol, 290: H560-76, 2006.

Katavetin, P: VEGF inhibition and renal thrombotic microangiopathy. N Engl J Med, 359: 205-6; author reply 206-7, 2008.

Kawamura, H, Li, X, Harper, S J, Bates, D O & Claesson-Welsh, L: Vascular endothelial growth factor (VEGF)-A165b is a weak in vitro agonist for VEGF receptor-2 due to lack of coreceptor binding and deficient regulation of kinase activity. Cancer Res, 68: 4683-92, 2008.

Oltean, S, Neal, C. R., Salmon, A., Quaggin, S. E., Harper, S. J., Bates, D. O.: VEGF over-expression increases glomerular water permeability in vivo in a conditional and inducible mouse model. American Society of Nephrology. San Diego, 2009 (In Press).

Rennel, E S, Hamdollah-Zadeh, M A, Wheatley, E R, Magnussen, A, Schuler, Y, Kelly, S P, Finucane, C, Ellison, D, Cebe-Suarez, S, Ballmer-Hofer, K, Mather, S, Stewart, L, Bates, D O & Harper, S J: Recombinant human VEGF165b protein is an effective anti-cancer agent in mice. Eur J Cancer, 44: 1883-94, 2008.

Rostgaard, J, Qvortrup, K.: Sieve plugs in fenestrae of glomerular capillaries—site of the filtration barrier? Cells Tissues Organs, 170: 132-138, 2002.

Satchell, S C & Braet, F: Glomerular endothelial cell fenestrations: an integral component of the glomerular filtration barrier. Am J Physiol Renal Physiol, 296: F947-56, 2009.

Satchell, S C, Tasman, C H, Singh, A, Ni, L, Geelen, J, von Ruhland, C J, O'Hare, M J, Saleem, M A, van den Heuvel, L P & Mathieson, P W: Conditionally immortalized human glomerular endothelial cells expressing fenestrations in response to VEGF. Kidney Int, 69: 1633-40, 2006.

Savin, V J & Terreros, D A: Filtration in single isolated mammalian glomeruli. Kidney Int, 20: 188-97, 1981

### INDUSTRIAL APPLICABILITY

The present invention provides a new family of active agents for use in treating or preventing microvascular hyperpermeability disorders, or in regulating the pro-angiogenic pro-permeability properties of VEGFₓₓₓ isoforms, or in supporting epithelial cell survival without increased permeability, or in reducing the nature (for example the number density and/or size) of fenestrations of epithelial filtration membranes.

This activity of the VEGFₓₓₓb family of proteins, and particularly VEGF₁₆₅b, is unexpected in view of the known properties of the proteins.

This finding opens up many new therapeutic and other treatments of human and animal subjects suffering from or susceptible to microvascular hyperpermeability disorders.

SEQUENCE DATA
SEQ. ID. NO: 1
TCA GCG CAG CTA CTG CCA TC
SEQ. ID. NO: 2
GTG CTG GCC TTG GTG AGG TT
SEQ. ID. NO: 3
ACG TCC TAA GCC AGT GAG TG
SEQ. ID. NO: 4
CAG CCT TCT CAG CAT CAG TC
SEQ. ID. NO: 5
ACA AGA TCC GCA GAC GTG TA
SEQ. ID. NO. 6
ACA GAT GGC TGG CAA CTA GA
SEQ. ID NO. 7
AAA ACC TTT TGT TGC TTT GGA
SEQ. ID NO. 8
GAA ATG GGA TTG GTA AGA TGA
SEQ. ID NO. 9
GGC AGC TTG AGT TAA ACG AAC G
SEQ. ID NO. 10
ATG GAT CCG TAT CAG TCT TTC CTG C

1-28. (canceled)

29. A method of treating a microvascular hyperpermeability disorder, or regulating the pro-angiogenic pro-permeability properties of VEGFₓₓₓ isoforms, or supporting epithelial cell survival without increased permeability, or reducing the nature of fenestrations of epithelial filtration membranes, the method comprising:
administering to a subject or to an epithelial filtration membrane an effective amount of a VEGFₓₓₓb active agent.

30. The method according to claim 29, wherein the VEGFₓₓₓb active agent selectively promotes the presence or expression of VEGFₓₓₓb in preference to VEGFₓₓₓ in cells.

31. The method according to claim 29, wherein the VEGFₓₓₓb active agent is VEGFₓₓₓb or an agent which selectively promotes the presence or expression of VEGFₓₓₓb in preference to VEGFₓₓₓ in cells.

32. The method according to claim 29, wherein the VEGFₓₓₓb active agent is an expression vector system expressing a VEGFₓₓₓb active agent.

33. The method according to claim 29, wherein the VEGFₓₓₓb active agent comprises one or more of VEGF₁₆₅b, VEGF₁₈₉b, VEGF₁₄₅b, VEGF₁₈₃b and VEGF₁₂₁b.

34. The method according to claim 29, wherein the VEGFₓₓₓb comprises VEGF₁₆₅b.

35. A method of reducing the permeability of a microvascular membrane, or regulating the pro-angiogenic pro-permeability properties of VEGFₓₓₓ isoforms, or supporting epithelial cell survival without increased permeability, or reducing the nature of fenestrations of epithelial filtration membranes, the method comprising:
contacting the membrane with an effective amount of a VEGFₓₓₓb active agent.

36. The method according to claim 35, wherein the VEGFₓₓₓb active agent selectively promotes the presence or expression of VEGFₓₓₓb in preference to VEGFₓₓₓ in cells.

37. The method according to claim 35, wherein the VEGFₓₓₓb active agent is VEGFₓₓₓb or an agent which selectively promotes the presence or expression of VEGFₓₓₓb in preference to VEGFₓₓₓ in cells.

38. The method according to claim 35, wherein the VEGFₓₓₓb active agent is an expression vector system expressing a VEGFₓₓₓb active agent.

39. The method according to claim 35, wherein the VEGFₓₓₓb active agent comprises one or more of VEGF₁₆₅b, VEGF₁₈₉b, VEGF₁₄₅b, VEGF₁₈₃b and VEGF₁₂₁b.

40. The method according to claim 35, wherein the VEGFₓₓₓb comprises VEGF₁₆₅b.

41. A method of testing a subject for risk or susceptibility to microvascular hyperpermeability disorders, disorders of regulation of the pro-angiogenic pro-permeability properties of VEGFₓₓₓ isoforms, disorders of epithelial cell survival and permeability, and/or disorders in the nature of fenestrations of epithelial filtration membranes, the method comprising:
obtaining a biological sample from the subject, and
assaying the levels of VEGFₓₓₓb in the sample relative to normal absolute VEGFₓₓₓb levels or relative to normal VEGFₓₓₓb: VEGFₓₓₓ ratio.

42. A method of testing a subject for risk or susceptibility to microvascular hyperpermeability disorders, disorders of regulation of the pro-angiogenic pro-permeability properties of VEGFₓₓₓ isoforms, disorders of epithelial cell survival and permeability, and/or disorders in the nature of fenestrations of epithelial filtration membranes, the method comprising:
obtaining a biological sample from the subject, and
genotyping the sample to determine a risk of underexpressing VEGFₓₓₓb relative to normal absolute VEGFₓₓₓb level or relative to normal VEGFₓₓₓb: VEGFₓₓₓ ratio.

43. A method of supporting epithelial cell survival or treating a disorder resulting from increased epithelial cell degeneration or decreased epithelial survival, the method comprising:
administering to a subject or to an epithelial cell population an effective amount of a VEGFₓₓₓb active agent.

44. The method according to claim 43, wherein the VEGFₓₓₓb active agent selectively promotes the presence or expression of VEGFₓₓₓb in preference to VEGFₓₓₓ in cells.

45. The method according to claim 43, wherein the VEGFₓₓₓb active agent is VEGFₓₓₓb or an agent which selectively promotes the presence or expression of VEGFₓₓₓb in preference to VEGFₓₓₓ in cells.

46. The method according to claim 43, wherein the VEGFₓₓₓb active agent is an expression vector system expressing a VEGFₓₓₓb active agent.

47. The method according to claim 43, wherein the VEGFₓₓₓb active agent comprises one or more of VEGF₁₆₅b, VEGF₁₈₉b, VEGF₁₄₅b, VEGF₁₈₃b and VEGF₁₂₁b.

48. The method according to claim 43, wherein the VEGFₓₓₓb comprises VEGF₁₆₅b.


SEQUENCE LISTING (10 sequences)

SEQ ID NO: 1 - DNA, 20 bp, Artificial
  PCR Primer
tcagcgcagctactgccatc

SEQ ID NO: 2 - DNA, 20 bp, Artificial
  PCR Primer
gtgctggccttggtgaggtt

SEQ ID NO: 3 - DNA, 20 bp, Artificial
  PCR Primer
acgtcctaagccagtgagtg

SEQ ID NO: 4 - DNA, 20 bp, Artificial
  PCR Primer
cagccttctcagcatcagtc

SEQ ID NO: 5 - DNA, 20 bp, Artificial
  RT-PCR Primer
acaagatccgcagacgtgta

SEQ ID NO: 6 - DNA, 20 bp, Artificial
  RT-PCR Primer
acagatggctggcaactaga

SEQ ID NO: 7 - DNA, 21 bp, Artificial
  PCR Primer
aaaaccttttgttgctttgga

SEQ ID NO: 8 - DNA, 21 bp, Artificial
  PCR Primer
gaaatgggattggtaagatga

SEQ ID NO: 9 - DNA, 22 bp, Artificial
  PCR Primer
ggcagcttgagttaaacgaacg

SEQ ID NO: 10 - DNA, 25 bp, Artificial
  PCR Primer
atggatccgtatcagtctttcctgc